(19) United States
(12) Patent Application Publication
(10) Pub. No.: US 2019/0105519 A1
(43) Pub. Date: Apr. 11, 2019

(54) ULTRASOUND DIRECTED CAVITATIONAL METHODS AND SYSTEM FOR OCULAR TREATMENTS

(71) Applicant: ALeyeGN Technologies LLC, Saratoga, CA (US)

(72) Inventors: Rajeev HEREKAR, Palo Alto, CA (US); Satish HEREKAR, Palo Alto, CA (US)

(21) Appl. No.: 15/947,134

(22) Filed: Apr. 6, 2018

Related U.S. Application Data
(63) Continuation of application No. PCT/US2016/055829, filed on Oct. 6, 2016.
(60) Provisional application No. 62/237,840, filed on Oct. 6, 2015.
(60) Provisional application No. 62/254,138, filed on Nov. 11, 2015.
(60) Provisional application No. 62/305,996, filed on Mar. 9, 2016.
(60) Provisional application No. 62/310,644, filed on Mar. 18, 2016.

(51) Int. Cl. A61N 7/02; A61B 8/10; A61B 3/10; A61B 8/00; A61B 3/00; A61F 9/00

(52) U.S. Cl. CPC A61N 7/02; A61B 8/10; A61B 3/102; A61N 2007/0078; A61B 3/0058; A61F 9/00; A61B 8/463

(57) ABSTRACT

Methods and system provide a focused spot having a cross-sectional size within a range from about 50 um to about 200 um full width half maximum (FWHM); the corresponding cavitation can be similarly sized within similar ranges. The ultrasound beam can be focused and pulsed at each of a plurality of locations to provide a plurality of cavitation zones at each of the target regions. Each pulse may comprise a peak power within a range generating focal negative peak pressures within a range from about 10 MPa to about 80 MPa. While the treatment pulses can be arranged in many ways within a region, in many instances the pulses can be spaced apart within a region to provide intact tissue such as intact sclera between pulses.

### CROSS-REFERENCE

The present application is a continuation of PCT Application PCT/US2016/055829, filed Oct. 6, 2016, entitled “ULTRASOUND DIRECTED CAVITATIONAL METHODS AND SYSTEMS FOR OCULAR TREATMENTS” (attorney docket no. 48848-704.601); which claims the benefit of U.S. provisional application 62/237,840, filed on 6 Oct. 2015, entitled “ULTRASOUND DIRECTED CAVITATIONAL METHODS AND SYSTEMS FOR OCULAR TREATMENTS” (attorney docket no. 48848-704.101); U.S. provisional application 62/254,138, filed on 11 Nov. 2015, entitled “ULTRASOUND DIRECTED CAVITATIONAL METHODS AND SYSTEMS FOR OCULAR TREATMENTS” (attorney docket no. 48848-704.102); U.S. provisional application 62/305,996, filed on 9 Mar. 2016, entitled “ULTRASOUND DIRECTED CAVITATIONAL METHODS AND SYSTEMS FOR OCULAR TREATMENTS” (attorney docket no. 48848-704.103); and U.S. provisional application 62/310,644, filed on 18 Mar. 2016, entitled “ULTRASOUND DIRECTED CAVITATIONAL METHODS AND SYSTEMS FOR OCULAR TREATMENTS” (attorney docket no. 48848-704.104); the entire disclosures of which are incorporated herein by reference.

### BACKGROUND OF THE INVENTION

Prior methods and system for treating tissue to increase elasticity can be less effective than would be ideal. For example, prior methods and system to increase tissue elasticity can include laser and thermal treatments such as pulsed lasers and radiofrequency (RF) treatment. Although some of these prior methods and systems may treat tissue to increase elasticity, this effect can be lost over time. This regression of the treatment effect can make the prior methods and system less than ideal for treatments such as elastomodulation of the eye for treatment of presbyopia and glaucoma. Also, such laser and RF based treatments can be somewhat more complex and expensive than would be ideal, such that fewer people can receive beneficial treatments.

Although ultrasound has been used previously with lithotripsy to ablate tissue, the prior ultrasonic devices can be less than ideally suited to treat tissue so as to increase the elasticity of tissue. Also, the prior ultrasound methods and system can be less than ideally suited to treat small structures such as tissues of the eye. For example, prior frequency used to lithotripsy methods and system can provide more heating than would be ideal, and the ultrasound beam may not be focused to a sufficiently small region to treat delicate structures such as delicate structures of the eye.

### SUMMARY OF THE INVENTION

The presently disclosed systems in methods provide improved treatment of the eye with improved accuracy and can provide decreased amounts of healing in response to tissue treatment. Although reference is made to treatment of an eye, the methods and apparatus disclosed herein can be used to treat many types of tissue, and the methods and apparatus disclosed herein will find application in many fields, such as ophthalmology, urology, orthopedics and cardiology.

In many embodiments, an ultrasound transducer is configured to provide a high intensity focused ultrasound beam with a sufficiently low duty cycle to decrease heating of the tissue. The focused ultrasound beam comprises a plurality of pulses, in which each pulse comprises at least one acoustic cycle. Each pulse may comprise a plurality of acoustic cycles. A time between a first pulse and a subsequent pulse can be arranged such that heating of the tissue is decreased. The high intensity focused ultrasound (HIFU) pulses can be configured to generate temporary cavitation, which can be helpful for softening tissue. The intensity of the pulses and duty cycle can be configured in many ways and can be configured to soften tissue or to resect tissue depending on the type of treatment. In many embodiments, the beam is focused to a small spot size to provide localized tissue treatment to delicate structures of the eye. The low duty cycle and focused beam can be used to treat localized tissue with decreased amounts of healing in response to the treatment, and in many instances the tissue remains transparent without generating a visible perceptible artifact to the patient for an extended period of time subsequent to treatment for example one year subsequent to treatment. Alternatively, or in combination the system can be configured to resect tissue with the ultrasound treatment during surgery, such as resecting the anterior lens capsule prior to removing the lens of the eye for cataract surgery. With surgical procedures to remove tissue, the ultrasound beam intensity may optionally be set sufficiently high to provide visible cavitation of tissue. Alternatively or in combination, tissue removal can be provided with lower duty cycles to liquefy or emulsify tissue. In many embodiments, the tissue treatment is provided without a surgical incision, which decreases the invasiveness of the procedure and healing in response to the procedure.

In many embodiments, the system is configured to scan of a focused spot to target region of the eye with a plurality of pulses delivered to a plurality of locations of the target region. The system comprises an ultrasound transducer coupled to an imaging system to direct the pulses to target tissue structures in response to images provided by the imaging system. The imaging system may comprise an ultrasonic imaging system or an optical imaging system. The imaging system can be used to identify target tissue structures such that the user can identify target tissue structures on a display and input treatment parameters to treat the target tissue structures, for example with a touch screen display. The system can be configured to scan the spot in many ways, and may translate or rotate the ultrasound transducer or scan the beam the beam with a phased array ultrasound transducer, and combinations thereof. In many embodiments, the imaging systems comprises an ultrasound biomicroscope used in combination with the ultrasound transducer in order to image and treat tissue through optically non-transparent structures of the eye, such as the limbus, sclera and iris.

The ultrasound transducer and processor can be configured in many ways to provide many types of treatment, such as substantially non-thermal treatment, tissue softening, tissue resection, thermal treatment and non-thermal treatment. The system can be configured to treat many delicate tissue structures of the eye with decreased invasiveness and healing in response to treatment. The HIFU beam can be configured to treat floaters by liquefying or emulsifying the floaters. The system can be configured to treat vitreous structures coupled to the retina which may be related to retinal detachment, for example with softening of the vitreous structures coupled to the retina. The system can be configured to treat presbyopia, with a combination of softening treatment to the lens of the eye, treatment of the sclera to soften the sclera, treatment of the vitreous humor to soften structures of the vitreous humor, or treatment of the ora serrrata to facilitate movement of the lens within the capsule, in order to soften these structures of the eye related to accommodation. The system can be configured for use with cataract surgery to soften the lens to facilitate removal with suction through an incision. The system may also be configured to provide refractive correction of the eye, for example with thermal treatment of corneal tissue to reshape the corneal tissue to correct the refractive error of the eye. Additional treatments may be provided with the methods and apparatus disclosed herein.

In one aspect, a system for treating tissue of an eye comprises an ultrasound transducer array configured to generate a plurality of high intensity focused ultrasound (“HIFU”) pulses, and a processor coupled to the ultrasound transducer array and configured with instructions scan the plurality of pulses to a plurality of locations to treat the tissue of the eye. The plurality of high intensity focused ultrasound (“HIFU”) pulses comprise a negative acoustic pressure within a range from about 10 Mega Pascal (MPA) to about 80 MPA The processor is configured with instructions to treat the eye with the HIFU beam to soften the tissue with a temperature increase to more than about 50 degrees Centigrade.

The tissue may comprise transparent tissue. The processor may be configured with instructions to scan the ultrasound beam to the plurality of locations to soften the tissue without opacifying the tissue. The processor may be configured to soften a target region of tissue with the plurality of pulses. A duty cycle of the plurality of pulses within the target region may be within a range from about 0.1% to 1%. The focused spot may comprise a cross-sectional size within a range from 50 um to 200 um. The processor and the transducer array may be configured to overlap the plurality of pulses at the plurality of locations. The processor and the transducer array may be configured to deliver the plurality of pulses to the plurality of locations without overlapping. The high intensity focused ultrasound may comprise frequencies within a range from about 750 kHz to about 25 MHz and optionally within a range from about 5 MHz to about 20 MHz.

The transducer array and the processor may be configured to provide a plurality of pulses to a plurality of separate treatment regions separated by a distance. A duty cycle of each of the plurality of separate treatment regions may comprise a duty cycle less than a duty cycle of the transducer array. The plurality of separate regions may comprise a first treatment region receiving a first plurality of pulses and a second treatment region receiving a second plurality of pulses, wherein the treatment alternates between the first plurality of pulses to the first region and the second plurality of pulses to the second region to decrease a duty cycle of each of the plurality of treatment regions relative to the duty cycle of the transducer array in order to decrease treatment time of the first region and the second region.

The system may further comprise an imaging system to view an image of the eye during treatment, and a display coupled to the imaging system and the processor to show the image of the eye during treatment. The imaging system may comprise an optical coherence tomography system or an ultrasound bio-microscopy (UBM) system. The imaging system may comprise UBM. The ultrasound transducer array and the UBM may be arranged to detect field perturbation of the HIFU beam within a field of view of the UBM. The processor and the display may be configured to visibly display the field perturbation on a real time image of the eye shown on the display. The display and the processor may be configured to show a plurality of targeted treatment regions on the image of the eye on the display prior to treatment with the HIFU beam. The processor may be configured to scan the focused HIFU beam to the plurality of targeted tissue regions. Optionally, the processor may be configured with instructions to display the image of the eye to view the image of the eye and define a pre-determined treatment region to treat the tissue with the plurality of pulses.

The system may further comprise a display coupled to the processor to show the image of the eye prior to treatment. The processor may be configured with instructions to receive user inputs to define the plurality of targeted tissue regions on the image of the eye prior to treatment with the ultrasound pulses. The processor may be configured with instructions to register the plurality of target tissue regions defined prior to treatment with a real time image of the eye acquired during the treatment and to show the target tissue regions of the eye in registration with the real time image of the eye. The imaging system may be aligned with the ultrasound transducer array. The processor may comprise instructions to direct the plurality of pulses to the plurality of treatment regions in response to registration of the real time image of the eye with the image of the eye in response to movement of the eye. The processor may be configured to scan the ultrasound beam to the plurality of locations through an optically non-transparent region of the eye, the region comprising one or more of an iris, a sclera or a limbus of the eye. The imaging system may comprise an ultrasound imaging system and the plurality of treatment regions may be visible on the display and imaged with the ultrasound imaging system through the optically non-transparent region of the eye. The target tissue region may optionally comprise transparent tissue.

The processor may be configured to scan the ultrasound beam to a plurality of locations. The transducer array may comprise a phased array configured to scan the ultrasound beam to the plurality of locations. The system may optionally further comprise an actuator coupled to the ultrasound array to scan the ultrasound beam to the plurality of locations.

The transducer array may be configured to focus the spot to provide a negative pressure within a range from about 10 MPA to about 50 MPA.

The transducer and the processor may be configured to focus the spot to a plurality of locations to soften the tissue with an increase in temperature of no more than about five degrees Centigrade.

The system may be configured to focus the spot to a plurality of locations to soften the tissue with an increase in temperature of no more than about five degrees Centigrade.

The processor and the ultrasound array may be configured to decrease a modulus of the tissue by at least about 5% without inducing substantial increase in light scatter of the tissue. Optionally, the increase light scatter of the tissue may be increased by no more than about 5% as measured with a Scheimpflug camera. Optionally, the light scatter may increase no more than about 1% as measured with Scheimpflug camera. The increase in light scatter may be measured pre-operatively and post-operatively.

The processor and the transducer array may configured to decrease a modulus of the tissue by an amount within a range from about 1% to about 50%. The decrease in modulus may remain stable for at least about one week post treatment and optionally about one month post treatment and further optionally at least about six months post treatment.

The processor and the transducer array may be configured to soften the tissue without substantially changing the index of refraction. An amount of change of the index of refraction may comprise no more than about 0.05 pre-operatively relative to post operatively.

The processor and the transducer array may be configured to soften the tissue without substantially changing the index of refraction. An amount of change of the index of refraction may comprise no more than about 0.01 pre-operatively relative to post operatively.

The processor and the transducer array may be configured to decrease the modulus of the tissue by an amount within a range from about 1% to about 50% without inducing an opacification of the treatment region.

The processor and the transducer array are configured to focus the beam to a plurality of locations in a three dimensional pattern in the eye, The transducer array may be configured to focus the beam to a plurality of different locations along an axis of propagation along the ultrasound beam and/or a plurality of different locations transverse to the ultrasound beam to define a three dimensional treatment region.

The processor may be configured with instructions to soften a lens of the eye to increase accommodation of the eye. The processor may be configured with instructions to soften a sclera of the eye, a vitreous humor of the eye, or a limbus of to increase accommodation of the eye.

The processor may be configured with instructions to treat floaters of the eye.

The processor may be configured with instructions to treat a refractive error of the eye with heating. The refractive error may comprise myopia, hyperopia, and/or astigmatism. The processor may be configured with instructions to treat the refractive error with a pattern of energy applied to a cornea of the eye to provide a temperature rise to at least about 50 degrees C. Treatment of refractive error may be combined with softening of tissue.

The system may further comprise a patient coupling structure configured to couple the eye to the ultrasound array.

The processor and the transducer array may be configured resect tissue with a three dimensional resection pattern.

The processor and the transducer array may be configured to spongify tissue, to mircoperforate tissue, and/or to emulsify tissue.

The processor and the transducer array may be configured to heat the tissue to greater 50 degrees centigrade to provide a thermal treatment.

The processor and the transducer array may be configured to provide a focused sub-surface treatment selected from the group consisting of myopia, hyperopia, astigmatism, presbyopia, spherical aberration, keratoconus (“KCN”), phacoemulsification, infective keratitis (“IK”), choroidal neovascularization (“CNV”), cyclo-sonocoagulation, glaucoma, floaters, vitreolysis/vitrectomy, lens epithelial cell (“LEC”) lysis, capsulorhexis, glistenings, tumor, sonothrombolysis/vascular obstruction, posterior corneal surface reshaping, posterior capsular opacification, capsular polishing, extravasation, posterior vitreous retinal detachment, posterior continuous curvilinear capsulotomy (“PCCC”), and/or anterior continuous curvilinear capsulotomy (“ACCC”).

The processor and the transducer array may be configured to direct the ultrasound beam through a tissue of the eye selected from the group consisting of a pupil, an epithelium, a conjunctiva, an iris, a capsule of a lens, a sclera, and a cornea.

In another aspect, a system to treat an eye comprises an ultrasound transducer to generate a HIFU beam and a processor coupled to the ultrasound transducer, the processor configured with instructions to generate the HIFU beam comprising a plurality of pulses. Each of the plurality of pulses comprises at least one acoustic cycle. Each pulse of the plurality of pulses is separated from a subsequent pulse of the plurality of pulses by a time within a range from about 1 microsecond to about 1000 microseconds in order to provide a duty cycle of no more than about 5 percent (%) to a target tissue region.

The duty cycle, number of cycles of each pulse, and/or negative acoustic pressure may be configured such that the tissue remains substantially transparent subsequent to treatment. Optionally, the tissue may be substantially transparent one month subsequent to treatment and optionally one year subsequent to treatment.

The duty cycle, number of cycles of each pulse, and/or negative acoustic pressure may be configured such that the tissue is substantially transparent within one minute of completing the ultrasound treatment.

The duty cycle, number of cycles of each pulse, and/or negative acoustic pressure may be configured such that the HIFU beam generates cavitation in the tissue. The tissue may be substantially transparent after the beam has treated the tissue.

The duty cycle, number of cycles of each pulse, and/or negative acoustic pressure may be configured such that the HIFU beam generates visible cavitation in tissue. The tissue may become transparent after the beam has treated the tissue. The cavitation may be visible with ultrasound bio-microscopy and/or optical coherence tomography.

The at least one acoustic cycle may comprise a plurality of acoustic cycles within a range from about 2 acoustic cycles to about 100 acoustic cycles, optionally within a range from about 3 acoustic cycles to about 50 acoustic cycles, and optionally within a range from about 4 acoustic cycles to about 25 acoustic cycles.

The processor may be configured with instructions so that the duty cycle for overlapping pulses is within a range from about 0.1% to about 4%, and optionally within a range selected from the group consisting of from about 0.2% to about 2%, within a range from about 0.4% to about 1% and from about 0.5% to about 0.7%.

The processor and transducer may be configured with instructions so that the negative acoustic pressure is within a range from about −10 Mega Pascal (MPa) to about −40 MPa in order to soften the tissue.

An acoustic lens is located along a path of the HIFU energy to focus the HIFU beam to the spot.

An acoustic lens may be located along a path of the HIFU energy to focus the HIFU beam to the spot, and the acoustic lens may be located along the path between the transducer and the spot.

The transducer may comprise a phased array transducer to focus the HIFU beam to the spot.

The system may further comprise a component to scan the spot the component selected from the group consisting of a phased array transducer, a one dimensional phased array transducer, a two dimensional phased array transducer, a translation stage, an X-Y translation stage, an actuator, a galvanometer and a gimbal.

The processor may be configured to scan the spot in a three dimensional pattern.

The processor may be configured to scan the spot in a pre-determined three dimensional pattern.

The processor may be configured with instructions to scan the spot to a plurality of locations with a plurality of overlapping sequential spots.

The processor may be configured with instructions to scan the spot to a plurality of locations with a plurality of non-overlapping sequential spots.

In another aspect, a method of treating an eye comprises generating a HIFU beam with an ultrasound transducer and directing the plurality of pulses to the tissue to soften the tissue of the eye with a temperature increase of no more than about 5 degrees Centigrade. The HIFU beam comprises a plurality of pulses, each of the plurality of pulses comprising at least one acoustic cycle. Each pulse of the plurality of pulses is separated from a subsequent pulse of the plurality of pulses by a time within a range from about 1 microsecond to about 1000 microseconds in order to provide a duty cycle of no more than about 5 percent (%) to a target tissue region. The HIFU beam may comprise a focused spot having a cross-sectional size within a range from about 10 um to about 1 mm. A pressure of the ultrasound beam may comprise a peak negative acoustic pressure within a range from about −10 Mega Pascal (MPA) to about −80 MPA in order to soften the tissue.

The tissue may remain substantially transparent subsequent to treatment. Optionally, the tissue may be substantially transparent one month subsequent to treatment and optionally one year subsequent to treatment.

The tissue may be substantially transparent within one minute of completing the ultrasound treatment.

The HIFU beam may generate cavitation in the tissue and wherein the tissue is substantially transparent after the beam has treated the tissue.

The HIFU beam may generate visible cavitation in tissue. The tissue may become transparent after the beam has treated the tissue. The cavitation may optionally be visible with ultrasound bio-microscopy and/or optical coherence tomography.

The at least one acoustic cycle may comprise a plurality of acoustic cycles within a range from about 2 acoustic cycles to about 100 acoustic cycles, optionally within a range from about 3 acoustic cycles to about 50 acoustic cycles, and optionally within a range from about 4 acoustic cycles to about 25 acoustic cycles.

The duty cycle for overlapping pulses may be within a range from about 0.1% to about 4%, and optionally within a range selected from the group consisting of from about 0.2% to about 2%, within a range from about 0.4% to about 1% and from about 0.5% to about 0.7%.

The negative acoustic pressure may be within a range from about −10 Mega Pascal (MPa) to about −40 MPa in order to soften the tissue.

An acoustic lens may be located along a path of the HIFU energy to focus the HIFU beam to the spot.

An acoustic lens may be located along a path of the HIFU energy to focus the HIFU beam to the spot, and the acoustic lens is located along the path between the transducer and the spot.

The transducer may comprise a phased array transducer to focus the HIFU beam to the spot.

The spot may be scanned with a component selected from the group consisting of a phased array transducer, a one dimensional phased array transducer, a two dimensional phased array transducer, a translation stage, an X-Y translation stage, an actuator, a galvanometer and a gimbal.

The spot may be scanned in a three dimensional pattern.

The spot may be scanned in a pre-determined three dimensional pattern.

The spot may be scanned to a plurality of locations with a plurality of overlapping sequential spots.

The spot may be scanned to a plurality of locations with a plurality of non-overlapping sequential spots.

In an aspect, a method of treating an eye comprises generating a HIFU beam with an ultrasound transducer array and scanning the HIFU beam in a pre-determined pattern to soften the tissue of the eye with a temperature increase of no more than about 5 degrees Centigrade. The HIFU beam comprises a focused spot at the treatment zone having a maximum cross-sectional dimension within a range from about 10 um to about 1 mm. A pressure of the ultrasound beam comprises a peak negative acoustic pressure within a range from about −10 Mega Pascal (MPA) to about −80 MPA in order to soften the tissue. The tissue remains substantially transparent subsequent to treatment.

The treated pattern may not produce an optically visible artifact to a patient viewing with the eye for a period of time post-treatment within a range from about one week post-treatment to about one month post treatment.

In another aspect, a system to treat tissue comprises an ultrasound transducer array and a processor coupled to the ultrasound transducer array, the processor comprising instructions to treat the tissue.

In another aspect, a system to treat a tissue of an eye comprises an ultrasound transducer array and a processor coupled to the ultrasound transducer array, the processor comprising instructions to treat one or more of a sclera, a cornea, a lens, a vitreous or zonulae extending between an ora serrata and a capsule of the lens of the eye.

In another aspect, a system to treat tissue comprises an ultrasound transducer array and a processor coupled to the ultrasound transducer array, the processor comprising instructions to resect the tissue, wherein the transducer array and the processor are configured to resect the tissue non-thermally with a focused high intensity ultrasound beam.

In another aspect, a system to treat tissue comprises an ultrasound transducer array and a processor coupled to the ultrasound transducer array, the processor comprising instructions to resect the tissue. The transducer array and the processor are configured to resect the tissue non-thermally with a focused high intensity ultrasound beam.

In another aspect, a system to treat tissue comprises an ultrasound transducer array and a processor coupled to the ultrasound transducer array, the processor comprising instructions to treat the tissue. The transducer array and the processor are configured to decrease light scatter of the tissue.

In another aspect, a system to resect tissue comprises an ultrasound transducer array and a processor coupled to the ultrasound transducer array, the processor comprising instructions to treat the tissue, wherein the transducer array and the processor are configured to non-thermally resect the tissue with ultrasound pulses to a plurality of locations of the tissue, the ultrasound pulses comprising a duty cycle of no more than about 5% at each of the plurality of locations, and wherein the transducer array comprises a duty cycle of 50% or more for the non-thermal pulses.

In another aspect, a method of treating an eye comprises directing ultrasound energy to the eye with a transducer array.

In any of the methods or systems described herein, an ultrasound beam may be focused to a small spot size with a frequency within a range from about 5 to 15 MHz in order to provide focus at locations 1 mm or less below a surface of the eye.

In any of the methods or systems described herein, ultrasound energy may be delivered so as to generate cavitation and increase elasticity of the target tissue with heating of no more than about 10 degrees C. to adjacent tissue.

In any of the methods or systems described herein, the processor and the transducer are configure to focus the ultrasound beam to spot having a cross-sectional size within a range from about 50 um to about 200 um full width half maximum (FWHM).

In any of the methods or systems described herein, the array and processor may be configured to provide first wavelengths to image the eye at first frequencies and second wavelengths to treat the eye at second frequencies.

In any of the methods or systems described herein, the processor and the phased array may be configured to scan the HIFU beam to a plurality of locations.

In any of the methods or systems described herein, the array may be mounted on an arm to move the transducer array to a plurality of locations around the eye.

In any of the methods or systems described herein, the processor may be configured with instructions to scan the HIFU beam to a plurality of locations within a region of the sclera extending from near the ora serrata to the cornea and into the cornea.

In any of the methods or systems described herein, the processor may be configured with instructions to perform one or more of sclerotripsy, corneotripsy, or phacotripsy.

In any of the methods or systems described herein, the processor may be configured with instructions to treat one or more of a cornea, a sclera, a lens, a zonule extending from the ora serrata to the lens capsule, a vitreous of the eye, or an ora serrata of the eye.

In any of the methods or systems described herein, the processor coupled to the ultrasound array may be configured to provide a negative acoustic pressure of within a range from about −20 to about 80 MPa.

In any of the methods or systems described herein, the processor coupled to the ultrasound array may be configured to remove collagenous tissue of a tissue structure and leave the collagenous tissue structure substantially intact. An amount of removed tissue may be within a range from about 5% to about 20%.

Any of the methods or systems described herein, may further comprise a first array to treat the tissue with HIFU and a second ultrasound array to image the eye.

In any of the methods or systems described herein, the array may comprise a phased array to focus high intensity ultrasound having frequencies within a range from about 5 MHz to about 15 MHz to the target location.

In any of the methods or systems described herein, the array and processor may be configured to resect tissue substantially without visible bubble formation. An amount of visible bubbles may comprise no more than 5% of a treatment volume. An amount of visible bubbles may comprise no more than 1% of a resected tissue treatment volume. An amount of visible bubbles comprises no more than 0.1% of a resected tissue treatment volume.

In any of the methods or systems described herein, the transducer array and the processor may be configured to non-thermally resect the tissue with ultrasound pulses to the plurality of locations of the tissue, the ultrasound pulses comprising a duty cycle of no more than about 3% at each of the plurality of locations, and wherein the transducer array comprises a duty cycle of 80% or more for the non-thermal pulses.

In any of the methods or systems described herein, the transducer array and the processor may be configured to non-thermally resect the tissue with ultrasound pulses to the plurality of locations of the tissue to define a plurality of tissue pieces with a plurality of tissue resection paths.

In any of the methods or systems described herein, the transducer array and the processor may be configured to non-thermally resect the tissue with ultrasound pulses to the plurality of locations of the tissue to define a plurality of tissue pieces with a plurality tissue resection paths, the plurality of tissue resection paths comprising a plurality of tissue perforations arranged to separate the tissue into the plurality of tissue pieces.

In any of the methods or systems described herein, the transducer array and the processor may be configured to non-thermally resect the tissue with ultrasound pulses to the plurality of locations of the tissue to define a three dimensional tissue resection pattern.

In any of the methods or systems described herein, the transducer array and the processor may be configured to non-thermally resect the tissue with ultrasound pulses to the plurality of locations of the tissue, and wherein the ultrasound pulses are configured to cleave collagen fibers with the non-thermal tissue resection.

In any of the methods or systems described herein, the transducer array and the processor may be configured to non-thermally resect the tissue with ultrasound pulses to the plurality of locations of the tissue. The ultrasound pulses may be configured to separate collagen fibers with the non-thermal tissue resection.

In any of the methods or systems described herein, the collagen fibers may comprise collagen fibers of one or more of a cornea, a limbus, a sclera, an iris, a lens capsule, a lens cortex, or zonulae.

In any of the methods or systems described herein, the plurality of pulses may be arranged to treat a refractive error of the eye, the refractive error comprising one or more of nearsightedness, farsightedness, astigmatism, aberration correction or wave-front aberration correction.

In any of the methods or systems described herein, the transducer array and the processor may be configured to non-thermally resect collagenous tissue with ultrasound pulses to the plurality of locations of the tissue arranged to define a piece of tissue corresponding a corrective lens for the eye. The ultrasound pulses may be arranged to allow the piece of tissue to be removed from the eye. Optionally, the pulses may be arranged to define an access path to the piece of tissue in order to perform a small incision lens extraction (SMILE). The tissue may optionally comprise corneal tissue.

In any of the methods or systems described herein, the transducer array and the processor may be configured to non-thermally separate collagenous tissue along a path with ultrasound pulses to the plurality of locations of the tissue arranged to separate the tissue into one or more layers along the path. The tissue may optionally comprise corneal tissue. The path may optionally define one or more of a corneal pocket, a corneal bed or a flap.

The ultrasound transducer array and the processor may be configured to transmit ultrasound energy through a corneal endothelium of the eye and focus the ultrasound beam away from the corneal endothelium in order resect tissue of the eye with the ultrasound beam and inhibit damage of the corneal endothelium.

In any of the methods or systems described herein, the ultrasound transducer array and the processor may be configured to transmit ultrasound energy through a corneal endothelium of the eye and focus the ultrasound beam away from the corneal endothelium in order resect tissue of the eye with the ultrasound beam and inhibit damage of the corneal endothelium. An amount of ultrasound energy delivered per unit area where the ultrasound beam is focused is within a range from about 1000 (one thousand) to about 100,000 (one hundred thousand) times greater than an amount of energy per unit area where the beam passes through the corneal endothelium.

In any of the methods or systems described herein, an amount of ultrasound energy delivered per unit area where the ultrasound beam is focused may be within a range from about 1,000 (one thousand) to about 100,000 (one hundred thousand) times greater than an amount of energy per unit area where the beam passes through an epithelial layer of one or more of a conjunctiva or a cornea of the eye.

In any of the methods or systems described herein, the transducer array may comprise a numerical aperture within a range from about 0.5 to about 10.

In any of the methods or systems described herein, the transducer array and processor may be configured to provide a plurality of pulses to a plurality of separate treatment regions. A duty cycle of each of the plurality of separate treatment regions may comprise a duty cycle less than a duty cycle of the transducer array.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts the structures of the eye with a treatment system, in accordance with embodiments;

FIG. 1B shows a blown up schematic of the ora serrata, in accordance with embodiments;

FIG. 1C shows an ultrabiomicroscpy image of the ora serrata, in accordance with embodiments;

FIG. 1D depicts a treatment setup for the ora serrata, in accordance with embodiments;

FIG. 2 shows a treatment system, in accordance with embodiments;

FIG. 3 shows a sclerotripsy treatment zone, in accordance with embodiments;

FIG. 4 shows a corneotripsy zone and a vitreotripsy zone, in accordance with embodiments;

FIG. 5 shows an annular phacotripsy zone, in accordance with embodiments;

FIG. 6 shows a multi depth phacotripsy zone with focused ultrasound, in accordance with embodiments;

FIG. 7 shows a HIFU array coupled to an imaging apparatus, in accordance with embodiments;

FIG. 8 shows another HIFU array coupled to an imaging apparatus, in accordance with embodiments;

FIGS. 9A-9B show a treatment zone for myopia, in accordance with embodiments;

FIGS. 10A-10B show a treatment zone for hyperopia, in accordance with embodiments;

FIGS. 11A1-11A2 show a treatment zone for astigmatism, in accordance with embodiments;

FIG. 11B shows an alternative treatment zone for astigmatism, in accordance with embodiments;

FIGS. 12A1-12A2 show a corneal treatment zone for presbyopia using a center near approach, in accordance with embodiments;

FIGS. 12B1 and 12B2 show a treatment zone for presbyopia using a center distance approach, in accordance with embodiments;

FIG. 12C shows a treatment zone for presbyopia using scleral erosion, in accordance with embodiments;

FIG. 12D shows a treatment zone for presbyopia including lenticular erosion, in accordance with embodiments;

FIG. 13 shows a treatment zone for keratoconus, in accordance with embodiments;

FIG. 14A shows treatment zones for phacoemulsification, in accordance with embodiments;

FIG. 14B shows a treatment zone for trans-corneal virtual phacotripsy, in accordance with embodiments;

FIG. 14C shows a treatment zone for phacotripsy, in accordance with embodiments;

FIG. 14D shows a patient coupling structure comprising a conic-shaped wall defining a fluidic well containing the HIFU transducer and degassed active pharmaceutical ingredient (API), in accordance with embodiments;

FIG. 14E shows another treatment zone for phacotripsy, in accordance with embodiments;

FIG. 15 shows focused pulse locations of a treatment zone for cyclo-sonocoagulation, in accordance with embodiments;

FIG. 16 shows focused pulse locations of a treatment zone for glaucoma, in accordance with embodiments;

FIG. 17 shows focused pulse locations of a treatment zone for floaters, in accordance with embodiments;

FIG. 18A shows a treatment zone for capsulorhexis, in accordance with embodiments;

FIG. 18B shows a treatment zone for posterior continuous curvilinear capsulotomy, in accordance with embodiments;

FIG. 18C shows a treatment zone for anterior continuous curvilinear capsulotomy, in accordance with embodiments;

FIG. 19 shows a focused pulse location of a treatment zone for glistenings, in accordance with embodiments;

FIG. 20 shows a treatment zone for sonothrombolysis/vascular obstruction, in accordance with embodiments;

FIG. 21 shows a treatment zone for posterior corneal surface, in accordance with embodiments;

FIG. 22A shows a treatment zone for posterior capsular opacification, in accordance with embodiments;

FIG. 22B shows a treatment zone for capsule polishing, in accordance with embodiments;

FIG. 22C shows another treatment zone for capsule polishing, in accordance with embodiments;

FIG. 22D shows a treatment zone for Soemmering's ring, in accordance with embodiments;

FIG. 22E shows a treatment zone for Elschnig's pearls, in accordance with embodiments;

FIG. 23 shows treatment zones for extravasation and occlusion, in accordance with embodiments;

FIG. 24A shows treatment zones for posterior vitreous retinal detachment, in accordance with embodiments;

FIG. 24B shows a tissue treatment zone comprising multiple non-adjacent treatment focal points, in accordance with embodiments;

FIG. 24C shows a tissue treatment zone comprising multiple adjacent treatment regions, in accordance with embodiments;

FIG. 25 shows the experimental setup utilized to generate the data presented in Table 3, in accordance with embodiments;

FIG. 26 shows treatment site locations described in Table 3, in accordance with embodiments;

FIG. 27 shows the results of Experiment 1, in accordance with embodiments;

FIGS. 28A-28C shows the results of Experiment 10, in accordance with embodiments;

FIG. 28A shows an ultrasound image used to monitor the effects of HIFU therapy, in accordance with embodiments;

FIG. 28B shows an ultrasound image of the eye during HIFU after cavitation has begun to occur, in accordance with embodiments;

FIG. 28C shows an ultrasound image of the eye later during HIFU treatment when cavitation has further accumulated, in accordance with embodiments;

FIGS. 29A-29D shows the results of Experiment 14, in accordance with embodiments;

FIG. 29A shows an ultrasound image used to monitor the effects of HIFU therapy, in accordance with embodiments;

FIG. 29B shows an ultrasound image of the eye during HIFU treatment prior to the generation of cavitation, in accordance with embodiments;

FIG. 29C shows an ultrasound image of the eye during HIFU after cavitation has begun to occur, in accordance with embodiments;

FIG. 29D shows an ultrasound image of the eye later during HIFU treatment when cavitation has further accumulated, in accordance with embodiments;

FIGS. 30A-30D shows the results of Experiment 16, in accordance with embodiments;

FIG. 30A shows an ultrasound image used to monitor the effects of HIFU therapy, in accordance with embodiments;

FIG. 30B shows an ultrasound image of the eye during HIFU treatment prior to the generation of cavitation, in accordance with embodiments;

FIG. 30C shows an ultrasound image of the eye during HIFU after cavitation has begun to occur, in accordance with embodiments;

FIG. 30D shows an ultrasound image of the eye later during HIFU treatment when cavitation has further accumulated, in accordance with embodiments;

FIGS. 31A-31D shows the results of Experiment 19, in accordance with embodiments;

FIG. 31A shows an ultrasound image used to monitor the effects of HIFU therapy, in accordance with embodiments;

FIG. 31B shows an ultrasound image of the eye during HIFU treatment prior to the generation of cavitation, in accordance with embodiments;

FIG. 31C shows an ultrasound image of the eye during HIFU after cavitation has just begun to occur, in accordance with embodiments;

FIG. 31D shows an ultrasound image of the eye later during HIFU treatment when cavitation has further accumulated, in accordance with embodiments;

FIG. 32A1 shows an eye treated at the lens, in accordance with embodiments;

FIG. 32A2 shows an OCT cross-sectional slice of the eye of FIG. 32A1, illuminating the cornea, in accordance with embodiments;

FIG. 32B1 shows another eye treated at the lens, in accordance with embodiments;

FIG. 32B2 shows an OCT cross-sectional slice of the eye of FIG. 32B1, illuminating the lens, in accordance with embodiments;

FIG. 32C1 shows another eye treated at the lens, in accordance with embodiments;

FIG. 32C2 shows an OCT cross-section of the eye of FIG. 32C1, in accordance with embodiments;

FIG. 32D1 shows another eye treated at the lens, in accordance with embodiments;

FIG. 32D2 shows an OCT cross-section of the eye of FIG. 32D1, in accordance with embodiments;

FIG. 33A1 shows an eye treated at the cornea, in accordance with embodiments;

FIG. 33A2 shows an OCT cross-sectional slice of the eye of FIG. 33A1, in accordance with embodiments;

FIG. 33B1 shows another eye treated at the cornea, in accordance with embodiments;

FIG. 33B2 shows an OCT cross-sectional slice of the eye of FIG. 33B1, in accordance with embodiments;

FIG. 33C1 shows yet another eye treated at the cornea, in accordance with embodiments;

FIG. 33C2 shows an OCT cross-sectional slice of the eye of FIG. 33C1, in accordance with embodiments;

FIG. 34 shows a treatment zone for phacotripsy, in accordance with embodiments;

FIG. 35 shows a schematic of a one-dimensional HIFU system, in accordance with embodiments;

FIG. 36 shows a schematic of a treatment system, in accordance with embodiments;

FIG. 37 shows a schematic of a display for use in directing treatment to targeted treatment zones, in accordance with embodiments;

FIG. 38 shows a flowchart of a method for determining a target treatment location, in accordance with embodiments;

FIG. 39A shows a schematic showing the formation and dissipation of microcavitation at a treatment pulse location over time, in accordance with embodiments; and

FIG. 39B shows a schematic of HIFU pulsing over time, in accordance with embodiments.

### DETAILED DESCRIPTION OF THE INVENTION

The methods and system disclosed herein are well-suited for treating many types of tissue such as tissue of the eye. The treated ocular tissue, or membranes or pathological transformations thereof, may comprise one or more of corneal tissue, lens tissue, scleral tissue, vitreal tissue, or zonulae extending between the lens capsule and the ora serrata.

Examples of treatment modalities of the eye suitable for use with the systems and/or methods disclosed herein are described in PCT/US2014/023763, filed on 11 Mar. 2014, entitled “SCLERAL TRANSLOCATION ELASTO-MODULATION METHODS AND APPARATUS” (attorney docket no. 48848-703.601); U.S. provisional application 62/237,840, filed on 6 Oct. 2015, entitled “ULTRASOUND DIRECTED CAVITATIONAL METHODS AND SYSTEMS FOR OCULAR TREATMENTS” (attorney docket no. 48848-704.101); U.S. provisional application 62/254,138, filed on 11 Nov. 2015, entitled “ULTRASOUND DIRECTED CAVITATIONAL METHODS AND SYSTEMS FOR OCULAR TREATMENTS” (attorney docket no. 48848-704.102); U.S. provisional application 62/305,996, filed on 9 Mar. 2016, entitled “ULTRASOUND DIRECTED CAVITATIONAL METHODS AND SYSTEMS FOR OCULAR TREATMENTS” (attorney docket no. 48848-704.103); and U.S. provisional application 62/310,644, filed on 18 Mar. 2016, entitled “ULTRASOUND DIRECTED CAVITATIONAL METHODS AND SYSTEMS FOR OCULAR TREATMENTS” (attorney docket no. 48848-704.104); the entire disclosures of which are incorporated herein by reference.

The methods and system disclosed herein provide improved methods and system for making tissue more elastic. Although specific reference is made to treatment of the eye, the methods and system disclosed herein can be used with many tissues, such treatment of tumors or thrombi inside or outside the eye.

The methods and apparatus disclosed herein are well suited for many ocular treatments. The methods and apparatus can be used to treat one or more of many disorders of the eye, and can be used to treat many of these disorders with a phased array, under control of computer instructions. The apparatus can be used to one or more of soften, resect, with non-thermal treatments, for example less than about 50 degree Centigrade (degrees C.). Alternatively or in combination the methods and apparatus can be used in a thermal mode to treat tissue thermally with treatments more than about 50 degrees C., for example about 60 degrees C. or more. The non-thermal treatment can be used in many ways, such as for accurate tissue resection. A phased array can be programmed to treat non-adjacent focal zones with a very high duty cycle, e.g. greater than about 50% from the phased array, while each of focal treatment zones has a duty cycle less than about 5%, for example 2.5% or less in order to provide non-thermal tissue resection with very high pulse repetition frequencies in order to decrease treatment time. The non-thermal tissue resection can be performed without substantial bubble formation, which allows the user such as a surgeon to accurately treat many regions of the eye, in many instances without interference from bubbles. In many instances, the mechanism of non-thermal treatment is substantially mechanical, such that tissue can be resected with very fine and accurate incision structures, which can be three dimensional. The phased array can also be used for imaging the tissue during treatment with imaging ultrasound from the array.

The methods and system disclosed herein can provide high intensity focused ultrasound (HIFU) treatment to tissue so as to increase elasticity of the tissue. The methods and system disclosed herein may utilize HIFU treatment to induce cavitation in a non-incisional and non-thermal manner. The HIFU-induced cavitation can focally disrupt or liquefy or micro-porate (spongify) tissue and reduce rigidity, thus enhancing both mobility of accommodative complexes and aqueous outflow facilities. By inducing cavitation non-thermally, the methods and system disclosed herein can provide improved safety over currently available thermal treatments.

Unlike laser treatment systems, HIFU tissue penetration is not dependent on the opacity of the tissue, therefore HIFU may have greater access to tissue than laser systems which cannot penetrate through opaque media. Additionally, by inducing cavitation non-thermally with HIFU, the methods and system disclosed herein may prevent boiling bubble formation during cavitation and subsequent opacification of treated tissue.

The increased elasticity of the tissue can be provided at locations arranged in order to provide a therapeutic effect, such as presbyopia or glaucoma treatment, with decreased amounts of regression. In many embodiments, the ultrasound beam can be focused to a small spot size with a frequency within a range from about 5 to 25 MHz (mega Hertz) in order to provide improved accuracy at shallow locations such as 1 mm or less below a surface of the eye, for example within a range from about 0.1 to about 0.9 mm. The energy can be delivered so as to generate cavitation and increase elasticity of the target tissue with decreased amounts of heat. In many instances, the ultrasound treatment provides debulking of the tissue which increases the elasticity of the tissue. The amount of heating of the treated tissue can be controlled to be no more than about 10 degrees C., for example no more than about 5 degrees C., which can increase elasticity with decreased amounts of regression.

The methods and system disclosed herein can provide a focused spot having a cross-sectional size within a range from about 50 um to about 200 um full width half maximum (FWHM); the corresponding cavitation can be similarly sized within similar ranges. The ultrasound beam can be focused and pulsed at each of a plurality of locations to provide a plurality of cavitation zones at each of the target regions. Each pulse may comprise a peak power within a range generating focal negative peak pressures of about 30 MPa (mega Pascals). While the treatment pulses can be arranged in many ways within a region, in many instances the pulses can be spaced apart within a region to provide intact tissue such as intact sclera between pulses. Alternatively or in combination, the pulses can be overlapped to provide an overlapping treatment regions or zones having dimensions within a range from about 100 um to about 1 mm, and a plurality of spaced apart treatment regions can be provided within a treatment location. The depth of the treatment can be controlled in accordance with the region being treated. For example, glaucoma treatments of Schlemm's canal can be about 0.5 mm or less, and treatment regions of the ora serrata which can be deeper, for example within a range from about 0.5 to about 1.0 mm deep. For treatments located along the ciliary apex the treatment can be within a range from about 0.25 mm to about 0.75 mm.

The methods and system disclosed herein can be used in many ways and can be used to image the tissue during treatment. Imaging may be configured to occur simultaneously with treatment. A processor can be coupled to the ultrasound array and configured with instructions to scan the beam to a plurality of locations and image the tissue during treatment. The system may also comprise a display coupled to the processor that allows the user to see the tissue treated on the display and to plan the treatment. The images shown on the display can be provided in real time and can allow the operator to accurately align the tissue with the treatment and may allow the operator to visualize the treatment area, and other locations away from the treatment area. The imaging of the treatment area can be used to identify the target area on the screen and to program the treatment depth and location in response to the images shown on the display. The imaging can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU and to image the eye with an embedded imaging apparatus, for example an optical coherence tomography (“OCT”) probe. The processor coupled to the array can be configured with instructions to provide both ultrasound wavelengths from the array. The imaging apparatus may provide additional tissue feedback data in real-time, for example temperature or elasticity.

The treated tissue such as tissue of the eye can be coupled to the ultrasound array in many ways. The ultrasound array can be coupled with one or more of a gel, a gel pack, water or trehalose.

The treatment system can be configured in many ways and may comprise a handheld probe, or a system with support structures to such as an arm and a base to couple to the eye.

The methods and systems disclosed herein may be used to treat presbyopia and/or glaucoma by reducing tissue stiffness in a target tissue using controlled cavitation-mediated ocular tissue erosion or fractionation (e.g. micro-debulking and thrombolysis). Tissue stiffness, for example rigidity in the corneal tissue and/or scleral tissue, may hinder movement of the ciliary apex forward, inward, or both. Stiffness may alternatively or in combination lead to reduced aqueous outflow, for example by causing compression of the non-porous Schlemm's canal. Treatment to reduce stiffness may include non-incisional and/or non-thermal methods, for example using ultrasound to induce cavitation in the tissue in order to focally disrupt, liquefy, of micro-porate (e.g. spongify) the tissue, or any combination thereof. A reduction in rigidity of the tissue may enhance the mobility of accommodative complexes such as the ciliary apes and/or improve the function of aqueous outflow pathways such as Schlemm's canal. Cavitation may be enhanced by injection of a gas into the ocular tissue of interest. Alternatively or in combination, a gas may be injected into the target treatment tissue in order to reduce the threshold of cavitation. The systems and methods disclosed herein may be used to treat the tissue of the eye for a number of pathologies and applications including one or more of regional lenticular debulking, deep eye infections, Bruch's membrane fenestrations, corneal flap making, and Uveal melanoma or edematous tissue debulking.

The use of ultrasound as described using a non-thermal or non-incisional treatment method may have a safety profile which easily permits repeated treatments or other follow-on surgeries

The present inventors have determined with both finite element analysis (FEA) and clinical outcomes analysis treatment regions suitable for debulking for the treatment of presbyopia and glaucoma. The treatment region can be located in stromal tissue of the cornea and can be about 0.25 to about 0.75 mm deep. The treatment can be located in the cornea and sclera, for example slightly below the epithelium and conjunctiva. Due to the benefits of sub-surface tissue debulking/softening, high frequency histotripsy transducers, such as preferably electronically steerable phased array 5 MHz-20 MHz HIFU transducers, can be used at under 250 W and with pulses within a range from 100 nsecs to 100 msec pulses. The pulse frequency can be under 1000 Hz repetition rates for sequential and non-sequential ocular treatments as described herein.

Using a customized deposition nomogram, temperature outside of the histotripsy focal zone may not exceed 50 degrees C. thus protecting tissue at depth. Negative acoustic pressures of up to −80 MPa (typically −30 MPa) can be provided and can be sufficient to provide a 10% debulking rate for a 360 degree treatment 3 minutes long.

As used herein an ultrasound pulse encompasses one or more cycles of acoustic oscillation comprising a positive ultrasound pressure and a negative ultrasound pressure.

The pulses can be configured in many ways, and may comprise a single oscillation, or a plurality of oscillations. The pulses can be configured with a low duty cycle or a higher duty cycle.

The treatment system may comprise in imaging apparatus such that the treatment can be combined imaging with one or more of magnetic resonance (MR) imaging, ultrasound biomicroscopy (“UBM”), ultrasound (“US”) imaging, optical coherence tomography (“OCT”), optical coherence elastography (“OCE”), or US elastography transducer measurements. The imaging apparatus can be combined with the HIFU treatment with either simultaneous oblique trans-iridional imaging or the coaxial therapeutic probe; and diagnostic images that are useful intra-operatively, for visualization as well as for feature/landmark tracking. Rapid real time MR images can be acquired when time-synchronized to HIFU histotripsy pulses with weighting motion gradients turned ON for greater cavitational sensitivity. MR/OCT/US guided histotripsy can include one or more of pretreatment planning, image-based alignment and siting of the HIFU focus, real-time monitoring of HIFU-tissue interactions, or real-time control of exposure and damage assessment.

Three or more types of treatments can be provided depending on HIFU settings: 1. liquefaction, 2. paste or 3. vacuolated thermal treatment. Liquefied treatment regions are pure mechanically-disrupted treatment regions and can be observed with a pulse duration less than 30 ms, which is slightly longer than time to boil. Paste treatments represent an intermediate state between mechanically-induced liquefaction and vacuolated thermal treatment. Paste treatment regions may be generated non-thermally, e.g. spongification, or thermally, as a pre-cursor state to vacuolated thermal treatment, or with a combination of both thermal and non-thermal settings. The use of chilled (4 degree C.) degassed Trehalose (optionally with NSAIDs) may be preferred over water as the coupling medium for improved ocular surface lubrication, in some embodiments.

The treatment system may comprise an imaging apparatus capable of determining tissue elasticity before, during, or after HIFU treatment, or some combination thereof, for example OCE or US elastography transducers. The treatment system may additionally or in combination comprise a mechanism for real-time temperature sensing, for example using an OCT transducer, in order for real-time monitoring of HIFU-induced temperature changes or to provide for control of HIFU exposure to maintain temperature.

Motorized diagnostic imaging in sync with histotripsy patterning can be achieved in these configurations. For example, real-time imaging of treatment tissue may allow for user input to a grid of target regions, which may be larger than the area covered by a single treatment or include multiple areas not in direct contact with each other, for motorized control of multiple treatments over a larger area, allowing the user to avoid manual repositioning which may save time and prevent mistakes.

The optional use of nanoparticles similar to nanoparticles for enhanced imaging can be used to enhance cavitation in some embodiments. Nanoparticles can be used with ultrasound treatment as disclosed herein to reduce the cavitational dosage requirements, for example by a factor of 2×-10×. The nanoparticles may comprise one or more of perfluorocarbon, lipid, albumin, or galactose, for example. Targeted (optionally drug-free) sonolysis due to microstreaming and micro-fragmentation (<5 um diameter) can improve micro-circulation and contains region of insonation demarcation with added safety. Treatments can be provided with decreased bleeding and decreased apoptosis, which can be shown with blood brain barrier and myocardial infraction studies, for example. While the nanoparticles can be used for any of the treatments disclosed herein such as glaucoma treatments, the nanoparticles can be beneficial for fractionation and apoptosis of choroidal neovascularization (“CNV”) and uveal melanomas, for example.

Ultrasound-assisted extravasation of the aqueous through the trabecular meshwork and Schlemm's canal can be provided with circumferential exposure with the potential for re-canalization and enhanced outflow with decreased damage due to ultrasound treatment. The treatment geometry can be arranged in many ways and may comprise a length within a range from 100 um to 1 mm, a volumetric regions within a range from (400 um×100 um×360 degrees) and durations of exposure of less than 3 minutes are easily managed with a motorized circumferential track and 5 MHz-10 MHz theranostic applicator.

Use of dual frequency histotripsy with a low frequency pump combined with high frequency ultrasound can be used to reduce the high frequency cavitation threshold.

Regions of treatment with ultrasound-generated cavitation may comprise one or more of the lens, the sclera, the posterior vitreous zonulae (“PVZ”), or the vitreous. The tissue may comprise diseased tissue such as pellucid marginal degeneration (“PMD”), or Choroidal Neovascularization (“CNV”), for example. Embodiments for US imaging with directed cavitation transducers provide for planning, guidance, or monitoring online for example. OCE with directed cavitation provide end points for lens, sclera, vitreous, PVZ end points during sonication. Surgical steps adjunct to scleratripsy, keratripsy and vitreotripsy are anticipated such as ocular chemotherapy, laser reshaping, translocation, hardening (cross-linking) or pocket cuts for small incision lens extraction of the cornea to correct refractive error of the eye, for example.

Drug delivery can be enhanced with the methods and apparatus disclosed herein. Debulking of tissue as disclosed herein can be used as a preparatory step and may be advantageously administered in combination drug delivery to promote drug delivery and improve delivery of the drug through the treated tissue.

The controlled cavitation as described herein can be provided with simultaneous imaging administered from the array to the tissue with a fluidic coupling path and a rotating arm to sequence the focused patterns within the tissue to deliver the drug at doses related to the scan rates of the ultrasound beam as disclosed herein.

The methods and apparatus can be configured in many ways to treat tissue. This system can be configured to generate one or more of liquefaction, or vacuolated tissue, for example. The ultrasound system can be configured to provide mechanical erosion of collagen with breaking of the collagen fibers with well-defined margins.

The following boiling histotripsy parameters and responses describe an upper treatment limit in accordance with examples. The treatment energy can be substantially lower than shown in Table 1 below. Alternatively, treatment parameters similar to those shown in Table 1 can be used with decreased amounts of time.

TABLE 1
Boiling Histotripsy parameters and histopathology responses

| | | | | | | | Border | | |
|---|---|---|---|---|---|---|---|---|---|
| | Pulse | | Duty | | | Protein | Destroyed | | Effect Within |
| | Duration | | Factor | PRF | | Denaturation | vs Intact | | Lesion |
| Liquefied | <30 | ms | <.02 | 1 | Hz | 0% | <40 | um | mechanical |
| Paste | <100 | ms | <0.2 | <2 | Hz | 22%-27% | <40 | um | mechanical & thermal |
| Vacuolated | >100 | ms | >0.2 | >2 | Hz | 70%-90% | <100 | um | thermal |

The tissue can be treated so as to provide small zones from which the tissue is removed by natural processes such as macrophages in order to remove tissue. The removed tissue can be removed from several small locations so as to make the tissue more elastic, similar to a sponge.

HIFU may be operated in mechanical mode to produce purely mechanical effects or in thermal mode to produce thermal effects in the tissue. Mechanical mode comprises a duty cycle of less than 2.5%, more preferably less than 1%. Thermal mode comprises a duty cycle of more than 2.5%. The device may be operated in either mechanical mode or thermal mode and may be readily switched between the two modes.

HIFU may be operated with a duty cycle range of about 0.1% to about 1% for cavitational histotripsy or a duty cycle range of about 1% to about 2.5% for boiling histotripsy depending on the energy applied by the HIFU system described herein. HIFU may be operated with a duty cycle range of about 0.01% to about 1% for cavitational histotripsy or a duty cycle range of about 1% to about 2.5% for boiling histotripsy depending on the energy applied by the HIFU system described herein.

The methods and system described herein may be operated with any combination of the parameters listed in Table 2.

TABLE 2
Treatment parameters.

| Parameter | Range | Preferred |
|---|---|---|
| HIFU frequency | 750 kHz to 25 MHz | 10 MHz |
| Total treatment | 0 min to 10 min | 4 min |
| duration | | |
| PRF | 1 Hz to 1000 Hz | 1000 Hz |
| Non-thermal | 0.1% to 2.5% | 1% |
| duty cycle | | |
| Negative | −10 MPa to −80 MPa | −30 MPa |
| acoustic | | |
| pressure | | |
| Tissue | 37° C. to 100° C. | 41° C. |
| temperature | | |
| Treatment size | 100 um × 400 um | May be configured |
| | (per focal point) | to scan and treat |
| | | multiple regions |
| | | with focal points |
| Treatment depth | 0 cm to 2.5 cm | 1 cm |
| Focal gain | 10 to 100 | Typical: 50 |

The HIFU system may be operated at a HIFU frequency within a range of about 750 kHz to about 25 MHz, for example within a range of about 1 MHz to about 25 MHz, preferably within a range of about 5 MHz to 15 MHz, more preferably within a range of about 5 MHz to 10 MHz, more preferably about 10 MHz. The HIFU frequency for example may be within a range of about 2 MHz to about 24 MHz, for example within a range of about 3 MHz to about 23 MHz or within a range of about 4 MHz to about 22 MHz. The frequency for example may be within a range of about 5 MHz to about 21 MHz, within a range of about 6 MHz to about 20 MHz, or within a range of about 7 MHz to about 19 MHz. The frequency may for example be within a range of about 8 MHz to about 18 MHz, within a range of about 9 MHz to about 17 MHz, or within a range of about 10 MHz to about 16 MHz. The frequency may for example be within a range of about 11 MHz to about 15 MHz, within a range of about 12 MHz to about 14 MHz, or within a range of about 10 MHz to about 13 MHz.

The total treatment duration may be up to 10 minutes, for example within a range from about 1 min to about 10 min, preferably about 4 min. The total treatment duration may for example be within a range of about 2 min to about 9 min, within a range of about 3 min to about 8 min, or within a range of about 4 min to about 7 min. The total treatment duration may for example be within a range of about 5 min to about 6 min. The total treatment duration may for example be within a range of about 2 min to about 6 min, preferably within a range of about 3 min to about 5 min, or within a range of about 4 min to about 6 min, and more preferably within a range of about 4 min to about 5 min. The total treatment duration for example may be within a range of about 3 min to about 10 min, or within a range of about 4 min to about 8 min.

The PRF of the HIFU system described herein may be within a range of about 1 Hz to about 1000 Hz, for example within a range of about 50 Hz to about 1000 Hz, preferably about 1000 Hz. The PRF may for example be within a range of about 100 Hz to about 900 Hz, within a range of about 200 Hz to about 800 Hz, or within a range of about 300 Hz to about 700 Hz. The PRF for example may be within a range of about 400 Hz to about 600 Hz, for example about 500 Hz to about 600 Hz. The PRF for example may be within a range of about 100 Hz to about 1000 Hz, preferably within a range of about 200 Hz to about 1000 Hz, more preferably within a range of about 500 Hz to about 1000 Hz.

The non-thermal duty cycle of the HIFU system described herein may be within a range of about 0.1% to about 2.5%, preferably less than 1.0%. The non-thermal duty cycle may for example be within a range of about 0.2% to about 2.4%, within a range of about 0.3% to about 2.3%, or within a range of about 0.4% to about 2.2%. The non-thermal duty cycle may for example be within a range of about 0.5% to about 2.1%, within a range of about 0.6% to about 2.0%, or within a range of about 0.7% to about 1.9%. The non-thermal duty cycle may for example be within a range of about 0.8% to about 1.8%, within a range of about 0.9% to about 1.7%, or within a range of about 1.0% to about 1.6%. The non-thermal duty cycle may for example be within a range of about 1.1% to about 1.5%, within a range of about 1.2% to about 1.4%, or within a range of about 1.2% to about 1.3%. The non-thermal duty cycle may for example be within a range of about 0.5% to about 1.5%, preferably within a range of about 0.7% to about 1.3%, more preferably within a range of about 0.8% to about 1.2%.

The non-thermal duty cycle of the HIFU system described herein may be within a range of about 0.01% to about 2.5%, preferably less than 1.0%. The non-thermal duty cycle may for example be within a range of about 0.01% to about 1%, within a range of about 0.02% to about 0.09%, or within a range of about 0.03% to about 0.08%. The non-thermal duty cycle may for example be within a range of about 0.04% to about 0.07% or within a range of about 0.05% to about 0.06%. The non-thermal duty cycle of the HIFU system described herein may be within a range of about 0.01% to about 2.5%, or within any range therebetween.

The number of cycles of the HIFU system described herein may be within a range of about 1 to about 100 cycles, for example about 10 to about 100 cycles. The number of cycles may be within a range of about 20 to about 100 cycles, for example about 30 to about 100 cycles, for example about 40 to about 100 cycles. The number of cycles may be within a range of about 50 to about 100 cycles, for example about 60 to about 100 cycles, for example about 70 to about 100 cycles. The number of cycles may be within a range of about 80 to about 100 cycles, for example about 90 to about 100 cycles. The number of cycles may be within a range of about 10 to about 50 cycles, for example about 10 to about 30 cycles. The number of cycles may be within a range of about 10 to about 80 cycles, for example about 20 to about 50 cycles.

The peak negative acoustic pressure of the HIFU system described herein may be within a range of about −10 MPa to about −80 MPa, preferably about −30 MPa. The negative acoustic pressure may for example be within a range of about −20 MPa to about −70 MPa, within a range of about −30 MPa to about −60 MPa, or within a range of about −40 MPa to about −50 MPa. The negative acoustic pressure may for example be within a range of about −10 MPa to about −50 MPa, preferably within a range of about −20 MPa to about −40 MPa, more preferably about −30 MPa.

The negative acoustic pressure of the HIFU system generated at the cornea may for example be calculated using the formula:

$${P}_{C} = {P}_{F} \frac{{A}_{F}}{{A}_{C}} \qquad ( 1 )$$

Where P_{C}=pressure at the cornea, P_{F}=pressure at the focal point of the HIFU energy, A_{F}=area of the focal point, and A_{C}=area of the cornea in line of the HIFU energy beam. The diameter of the focal point may for example be in a range from about 50 μm to 200 μm, thus the area of the focal point may be calculated to be about 1964 μm² to about 31416 μm². The negative pressure at the focal point may for example be in a range from about −10 MPa to about −80 MPa. The diameter of the cornea in the line of the HIFU beam may for example be about 3 mm, thus the area of the cornea may be about 7.07 mm². Using formula 1 to calculate the pressure at the cornea given the exemplary ranges described, the negative acoustic pressure at the cornea may for example be within a range of about 2.8 kPa to about 356 kPa.

The negative acoustic pressure at the cornea may for example be within a range of about 1 kPa to about 350 kPa, for example within a range of about 1 kPa to about 300 kPa. The negative acoustic pressure at the cornea may for example be within a range of about 1 kPa to about 250 kPa, for example about 1 kPa to about 200 kPa. The negative acoustic pressure at the cornea may for example be within a range of about 0 kPa to about 150 kPa, for example about 1 kPa to about 100 kPa. The negative acoustic pressure at the cornea may for example be within a range of about 1 kPa to about 50 kPa, for example about 1 kPa to about 10 kPa.

The temperature of the tissue may be within a range of about 37° C. to about 100° C., preferably 41° C. The temperature of the tissue may for example be within a range of about 37° C. to about 50° C., preferably within a range of about 37° C. to about 45° C., more preferably within a range of about 37° C. to about 44° C., still more preferably within a range of about 37° C. to about 41° C.

The treatment size per focal point may be about 100 um×400 um. The HIFU system described herein may be configured to scan and treat multiple regions with multiple focal points, thus the total treatment area may be any area of any size within the eye.

The treatment depth of the HIFU system described herein may be within a range of about 0 cm at the surface of the eye to about 2.5 cm deep within the eye, preferably about 1 cm depending on the target tissue. The treatment depth may for example be within a range of about 0.1 cm to about 2.4 cm, within a range of about 0.2 cm to about 2.3 cm, or within a range of about 0.3 cm to about 2.2 cm. The treatment depth may for example be within a range of about 0.4 cm to about 2.1 cm, within a range of about 0.5 cm to about 2.0 cm, or within a range of about 0.6 cm to about 1.9 cm. The treatment depth may for example be within a range of about 0.7 cm to about 1.8 cm, within a range of about 0.8 cm to about 1.7 cm, or within a range of about 0.9 cm to about 1.6 cm. The treatment depth may for example be within a range of about 1.0 cm to about 1.5 cm, within a range of about 1.1 cm to about 1.4 cm, or within a range of about 1.2 cm to about 1.3 cm. The treatment depth may for example be within a range of about 0.25 cm to 0.75 cm, within a range of about 0.5 cm to about 1.5 cm, or 0.5 cm or less. The treatment depth is determined by the location of the tissue being treated.

The focal gain of the HIFU system described herein may be within a range of about 10 to 100, for example within a range of about 20 to 90. The focal gain may for example be within a range of about 30 to 80, within a range of about 40 to 70, or within a range of about 50 to 60.

The voltage of the HIFU system described herein may be within a range of about 100V to about 400V, for example about 150V to about 350V. The voltage of the HIFU system described herein may be within a range of about 200V to about 300V, for example about 200V to about 250V.

The HIFU system described herein may generate a HIFU beam having a spot size, also referred to herein as a maximum dimension across (e.g. diameter), at the focal point. The spot size of the HIFU system described herein may be within a range of about 10 um to about 1 mm, or between any two values therebetween. The spot size may for example be within a range of about 25 um to about 400 um, or about 50 um to about 200 um, or about 100 um. The spot size may be within a range of about 60 um to about 190 um, about 70 um to about 180 um, or about 80 um to about 170 um. The spot size may be within a range of about 90 um to about 160 um, about 100 um to about 150 um, or about 110 um to about 140 um. The spot size may be within a range of about 120 um to about 130 um. The spot size may be within a range of about 10 um to about 900 um, for example within a range of about 50 um to about 850 um, or about 100 um to about 800 um. The spot size may be within a range of about 200 um to about 700 um, about 300 um to about 600 um, or about 400 um to about 500 um.

The HIFU system described herein may be used to non-thermally treat a tissue. Non-thermal treatment may cause a change in temperature of the treated tissue within a range of about 1 degree C. to about 5 degrees C., for example about 2 degrees C. to about 4 degrees C., or about 3 degrees C. The change in temperature may be within a range of about 2 degrees C. to about 5 degrees, or within about 3 degrees C. to about 4 degrees C. The change in temperature may within a range of about 3 degrees C. to about 5 degrees C., or about 4 degrees C. The change in temperature may be within a range of about 4 degrees C. to about 5 degrees C. The change in temperature may be within a range of about 1 degree C. to about 4 degrees C., for example about 1 degree C. to about 3 degrees C., or about 2 degrees C. The change in temperature may be about 1 degree C. or about 5 degrees C.

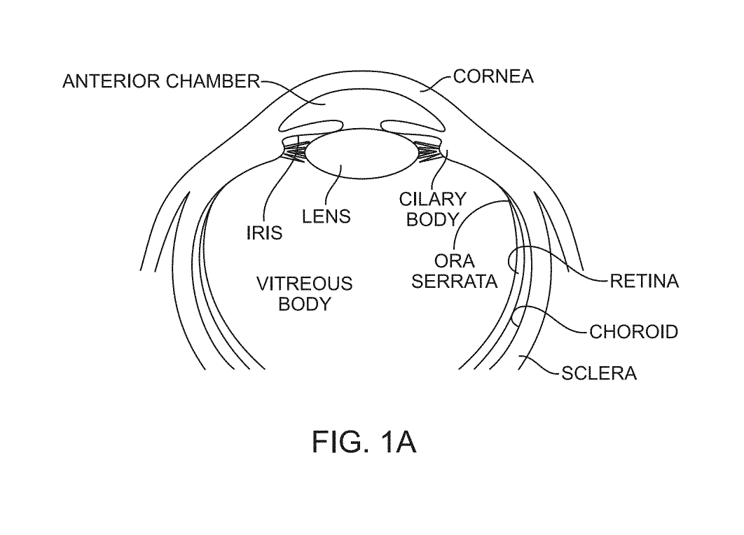

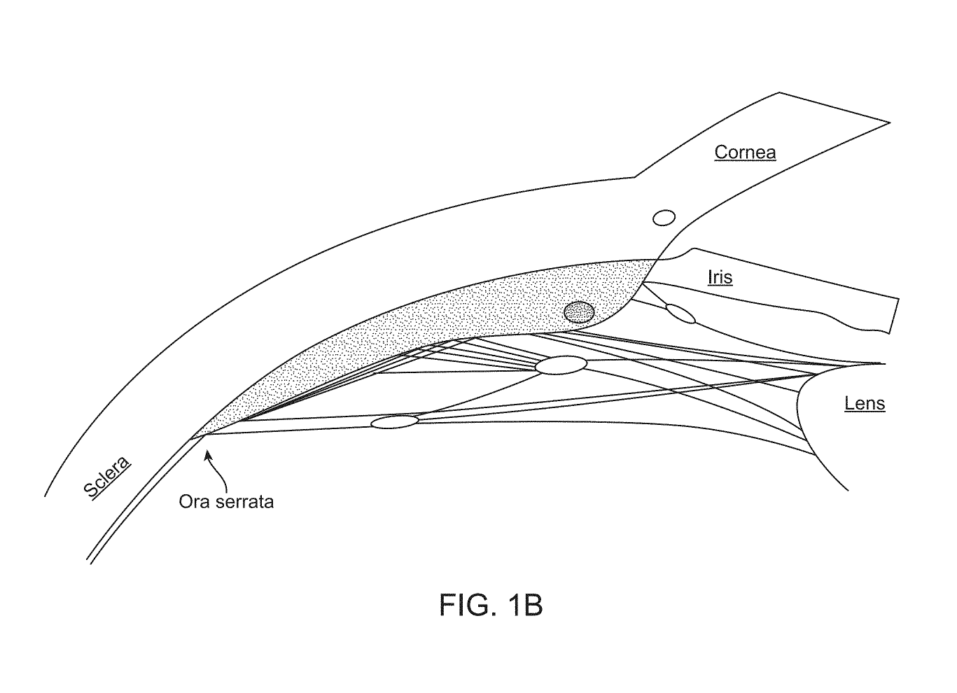

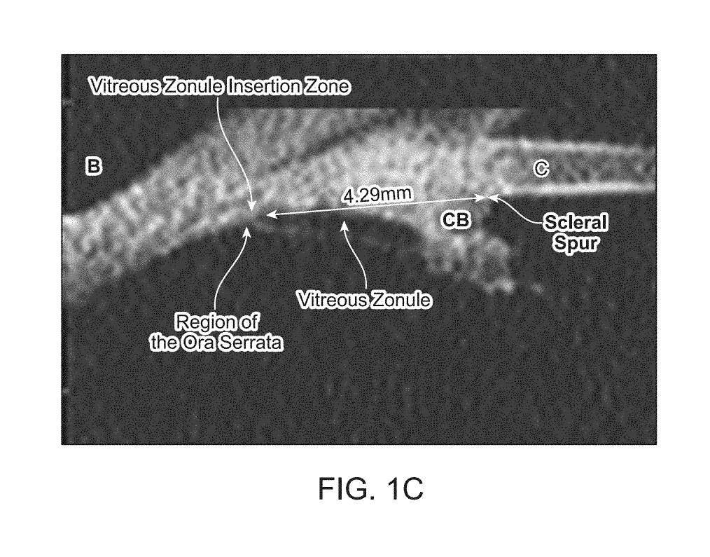

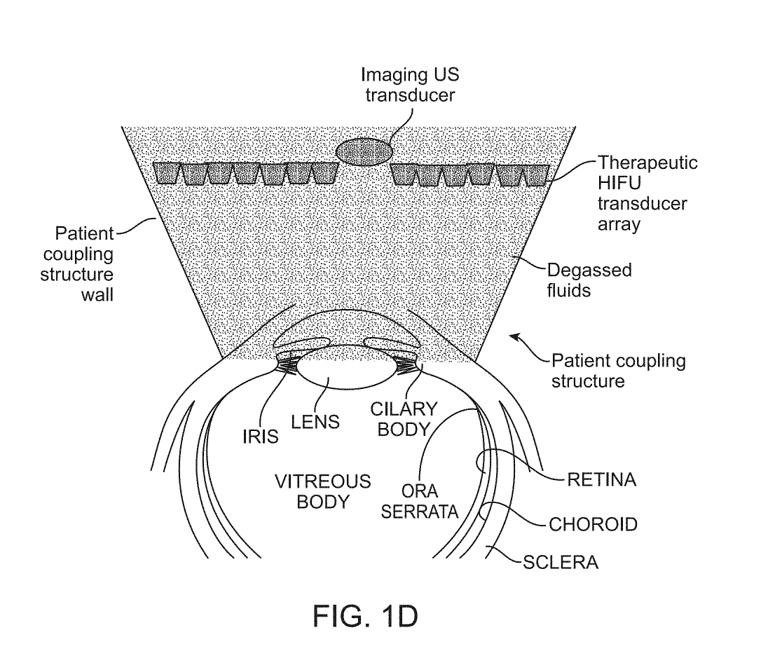

FIGS. 1A-1C depict the structures of the eye. Using the methods and system described herein, HIFU energy may be directed toward one or more of the structures depicted in FIG. 1A. As an example, FIG. 1B shows a blown up schematic of the ora serrata while FIG. 1C shows an ultrabiomicroscpy image of the ora serrata, one possible therapeutic target site. The ora serrata is about 440 um thick. FIG. 1D depicts a possible treatment setup for the ora serrata. In one embodiment, a therapeutic HIFU transducer array which comprises a centrally positioned imaging system, for example an ultrasound transducer or OCT fiber, is coupled to the eye with a patient coupling structure comprising a conic-shaped wall and a degassed fluid therein. The fluid interface may serve as a space to tightly focus the ultrasound beam to the desired treatment zone. Alternatively or in combination, the fluid may allow for greater control and/or a greater range over depth from the tissue surface. The fluid may further be used to control the temperature of surface tissue during exposure to HIFU during treatment. The fluid is preferably chilled to about 4° C. and may be one or more of a gel, a gel pack, water or trehalose. The HIFU array may be aimed at the cavitational zone appropriate for the treatment area, in this example the cavitational zone for the ora serrata is about 100 um in diameter and about 400 um deep. Using a low duty cycle, HIFU may be used to generate cavitation in the ora serrata.

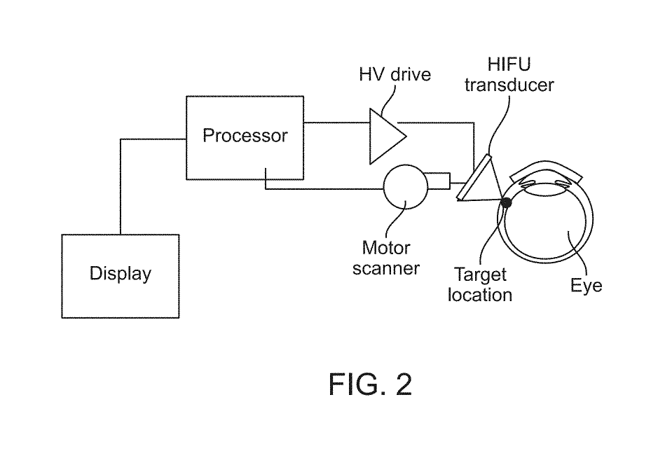

FIG. 2 shows a treatment system. The system comprises a HIFU array focusing ultrasound energy to a location inside the eye. A motor scanner can optionally be coupled to the ultrasound array to direct the treatment energy to the target locations of the eye. A processor may be coupled to a high voltage drive (HV) to drive the array. The processor can be coupled to the motor scanner to move the array during treatment. A display can be coupled to the processor to show the image of the eye as shown in FIG. 1. The image of the eye can be generated with imaging frequencies and wavelengths and the HIFU can be delivered to the eye with HIFU wavelengths as described herein.

Glaucoma Treatments

The methods and apparatus disclosed herein can be used to improve aqueous drainage from the eye to reduce intraocular pressure. The treatment region can be located anywhere on the aqueous outflow path between the trabecular meshwork and the outer surface of the conjunctiva. For example, a canal can be formed adjacent to Schlemm's canal. Schlemm's canal can be from about 300 to 350 um wide and 50 um tall if not blocked. The methods and apparatus can provide a channel above the canal (anterior) or along the canal, for example. The canal itself can be treated to remove blockage with the focused ultrasound beam as described herein. One or more channels can be created from Schlemm's canal to another tissue such as a lower layer of the conjunctiva to improve drainage. The HIFU beam as described herein can be used to dilate or open Schlemm's canal to improve outflow from the trabecular meshwork. The collector channels of Schlemm's canal or the trabecular meshwork can also be treated with the ultrasound as disclosed herein. Alternatively or in combination, the HIFU can be used to create channels similar to the collector channels.

Presbyopia Treatments

The presbyopia treatment can include ultrasound treatment of one or more of a scleral region, zonulae, vitreous, or the cornea. The PVZs may also be treated to improve the accommodative effect. The vitreous can also be treated, either with treatment of the zonulae or separately.

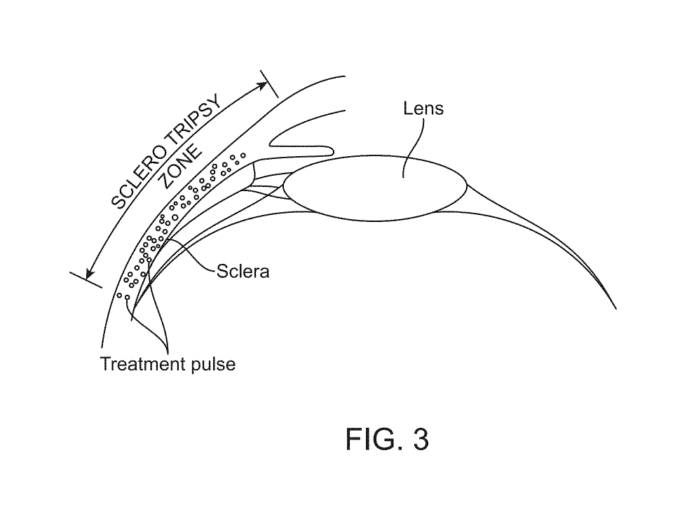

FIG. 3 shows a sclerotripsy treatment zone to treat presbyopia as described herein. The sclerotripsy treatment energy can be delivered at locations with treatment pulses as described herein.

The treatment along the cornea and sclera can extend at a depth of about 200 um below surface of the sclera and cornea, for example. The treatment of the cornea and sclera can extend from the sclera near the ora serrata to the cornea or into the cornea as described herein. Sclerotripsy may be used to augment scleral elasticity and spongify stromal tissue under cavitational control with targeted volumetric erosion. Erosion may occur without coagulation or damage to the conjunctiva or choroid using a HIFU transducer to disrupt the tissue mechanically and without heat.

While the HIFU to treat presbyopia can be directed to many locations, the presbyopia treatments as described herein can spare sclera, ciliary body, and retina.

The methods and apparatus disclosed herein to treat presbyopia can be used with accommodating intraocular lenses to improve accommodation with intraocular lenses.

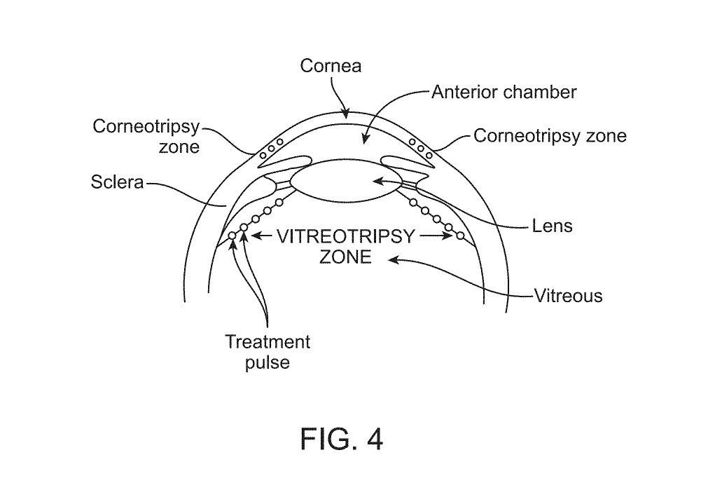

FIG. 4 shows a corneotripsy zone and a vitreotripsy zone.

The treatment of the cornea can provide improved movement of the cornea during accommodative effort, which may result in additional lens movement. The movement of the cornea may comprise an aspheric inward movement of the cornea in synch with movement of the pars plicata (near where sclera notch occurs). Inward movement of the cornea allows the lens equator to move inward and the lens to thicken and become more convex. Treatment of the sclera can have a similar effect, and the treatment region comprises a plurality of smaller treatment zones can extend along the sclera and cornea, for example with reference to FIG. 3. Treatment of the vitreous near the zonular insertion zone may improve accommodation by clearing thickened fibrous gel (also referred to herein as lacunae) and promoting forward movement. Treatment of the vitreous near the posterior pole may promote facile and stable shape changing of the lens during accommodation.

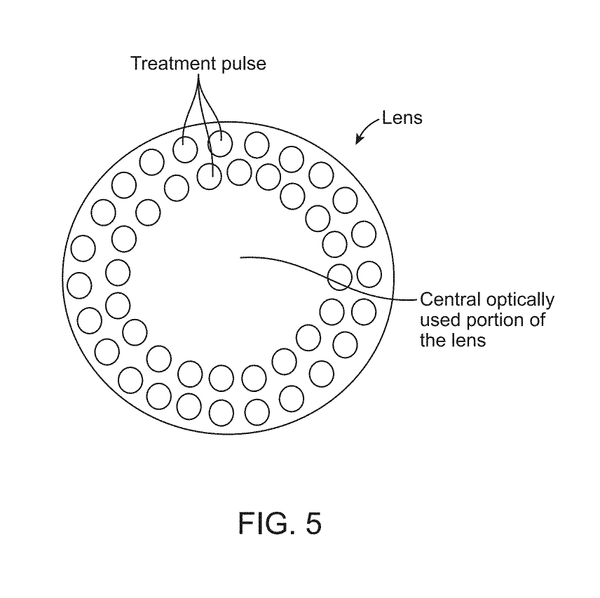

FIG. 5 shows an annular phacotripsy zone. The phacotripsy can improve accommodation of the lens and can be directed away from the central optically used portion of the lens. In one embodiment, HIFU treatment pulses may be directed to a series of treatment points within the lens such that treatment induces areas of increased elasticity in an annular pattern around the central optically used portion of the lens. Treatment may be guided by use of a mechanical motor or imaging system as described in FIG. 2 or any of the embodiments disclosed herein.

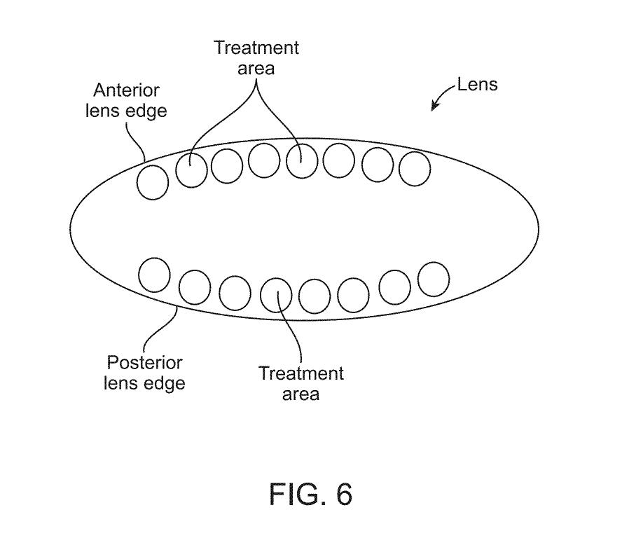

FIG. 6 shows a multi-depth phacotripsy zone with focused ultrasound, which may comprise an annular pattern similar to FIG. 5. A cross-section of the lens is shown. HIFU treatment pulses may also be directed to a series of treatment points at different depths within the lens. The zone depicted comprises a series of treatment areas on the anterior edge of the lens as well as a series of treatment areas on the posterior lens edge. This multi-focused approach may provide increased therapeutic benefit for relevant pathologies.

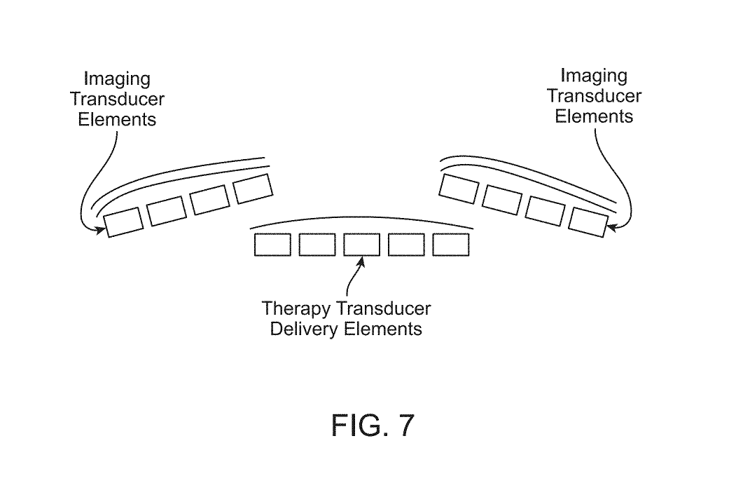

FIG. 7 shows an embodiment of a HIFU array coupled to an imaging apparatus. A pair of ultrasound imaging arrays and a HIFU array are arranged for real-time imaging during treatment. The imaging transducer elements and therapy transducer and elements can be coupled to the processor as disclosed herein. Coupling the imaging apparatus to the HIFU transducer allows for passive cavitation detection and imaging feedback to guide and inform treatment.

The HIFU transducer may comprise one or more of a phased array, a discrete array, an annular array, a spherical array, a spherical phased array, a focused array, or any combination thereof. The HIFU transducer may be combined with an imaging apparatus, for example embedded OCT sensors. Additionally, the transducer may be fabricated to allow for opto-acoustic excitation for precise theranostic delivery.

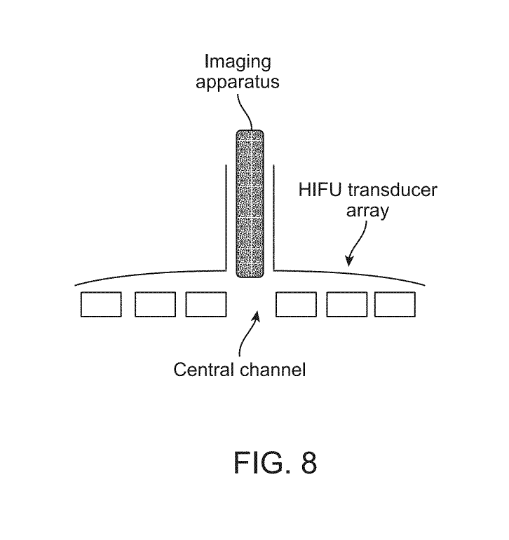

FIG. 8 shows another embodiment of a HIFU array coupled to an imaging apparatus. The HIFU array in this embodiment comprises a transducer with central channel in which the imaging apparatus may be disposed. For example, the imaging apparatus may be an OCT fiber optic cable. The OCT fiber may be disposed inside a channel extending from the center of the therapy transducer and can allow for real-time imaging of tissue at one or more times before, during, or after treatment with the HIFU array.

The HIFU array may be coupled to a number of imaging systems, including but not limited to MRI, UBM, ultrasound imaging, OCT, OCE, or US elastography.

The methods and system disclosed herein can be used to provide focused sub-surface treatments for a wide range of pathologies including myopia, hyperopia, astigmatism, presbyopia, spherical aberration, keratoconus (“KCN”), phacoemulsification, infective keratitis (“IK”), CNV, cyclo-sonocoagulation, glaucoma, floaters, vitreolysis/vitrectomy, lens epithelial cell (“LEC”) lysis, capsulorhexis, glistenings, tumor, sonothrombolysis/vascular obstruction, posterior corneal surface reshaping, posterior capsular opacification, capsular polishing, extravasation, posterior vitreous retinal detachment, posterior continuous curvilinear capsulotomy (“PCCC”), and anterior continuous curvilinear capsulotomy (“ACCC”). Treatments may be directed trans-pupil, trans-epithelium, trans-conjunctiva, trans-iris, trans-capsule, trans-sclera, trans-cornea, or any combination thereof as determined by the pathology or targeted zones.

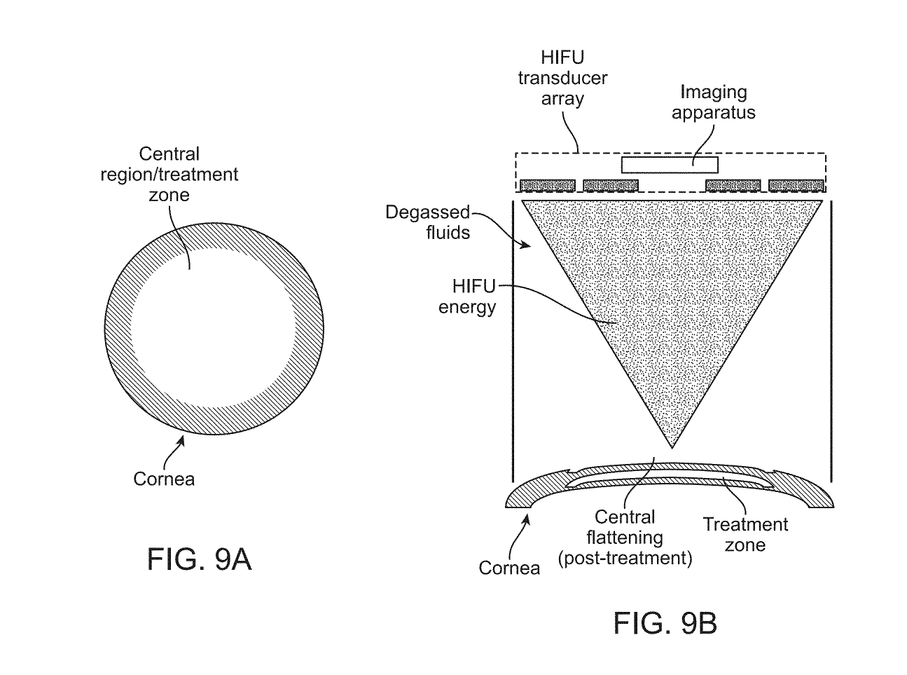

FIGS. 9A-9B show an embodiment of a treatment zone for myopia. Myopia, or nearsightedness, may occur when the cornea or the lens, or a combination of the two, is too curved relative to the length of the eye. The HIFU system described herein may be used to induce central flattening of the cornea to relieve myopia, for example. FIG. 9A shows the cornea en-face and FIG. 9B shows a cross-section of the cornea including the HIFU system. The transducer may be placed in front of the eye and focused to deliver HIFU energy through the epithelium into the corneal stroma for erosion. The calibrated transducer may be scanned to erode about 6 mm of the central region mechanically. Alternatively or in combination, the central region may shrunk thermally. An imaging apparatus such as OCT may provide feedback such as temperature, for example. The treatment deposition pattern may include a Munnerlyn pattern or an aspheric gradient pattern, effecting erosion of about 14 um deep to relieve myopia by about 1 Diopter. A single HIFU energy beam has been depicted here for simplicity, though it will be understood that the method described herein may comprise additional HIFU treatment beams and focal points to treat the central region.

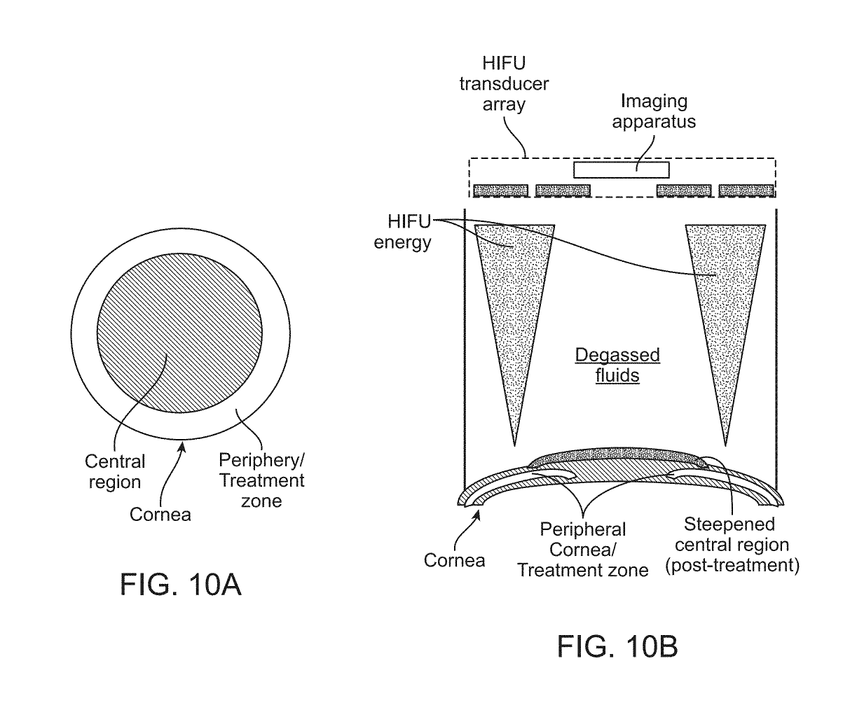

FIGS. 10A-10B show an embodiment of a treatment zone for hyperopia. Hyperopia, or farsightedness, may occur when the cornea has too little curvature relative to the length of the eye. The HIFU system described herein may be used to flatten the peripheral cornea in the peripheral region of the cornea about 5 mm to 9 mm from the center of the cornea. FIG. 10A shows the cornea en-face and FIG. 10B shows a cross-section of the cornea including the HIFU system. The peripheral cornea may be flatted by trans-epithelial mechanical erosion of the peripheral region. Alternatively or in combination the peripheral cornea may be flatted by trans-epithelial thermal shrinkage of the peripheral region. Such flattening may result in a relatively steeper central region of the cornea. HIFU may be deposited in a steepening pattern. Erosion of about 14 um deep may relieve hyperopia by about 1 Diopter. The HIFU beam may be directed to multiple locations using an electronically steered phased array or by mechanical motorized motion to treat multiple overlapping locations with the peripheral region. Two HIFU energy beams have been depicted here for simplicity, though it will be understood that the method described here may comprise additional HIFU treatment beams and focal points to treat the peripheral region.

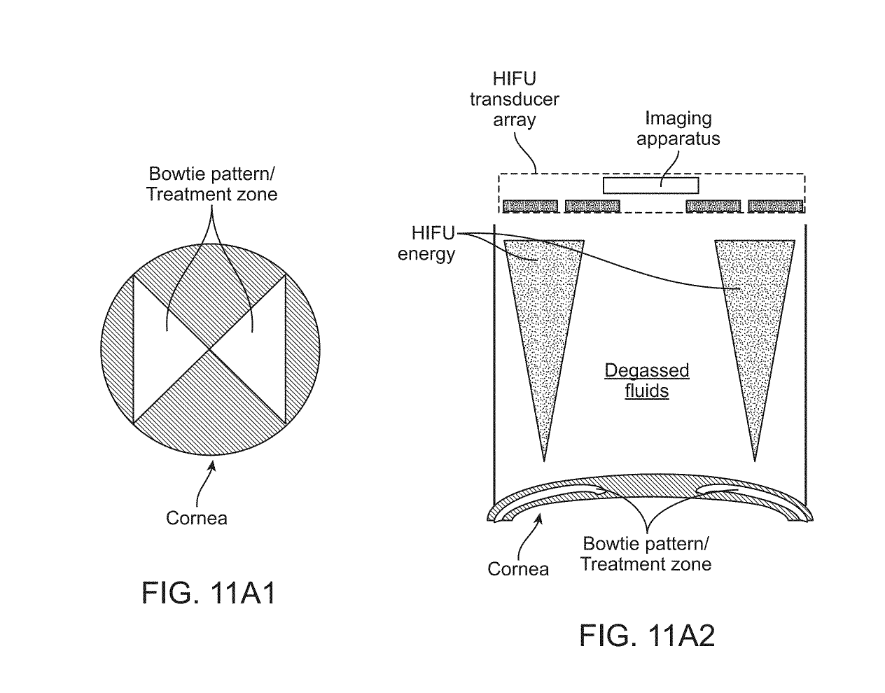

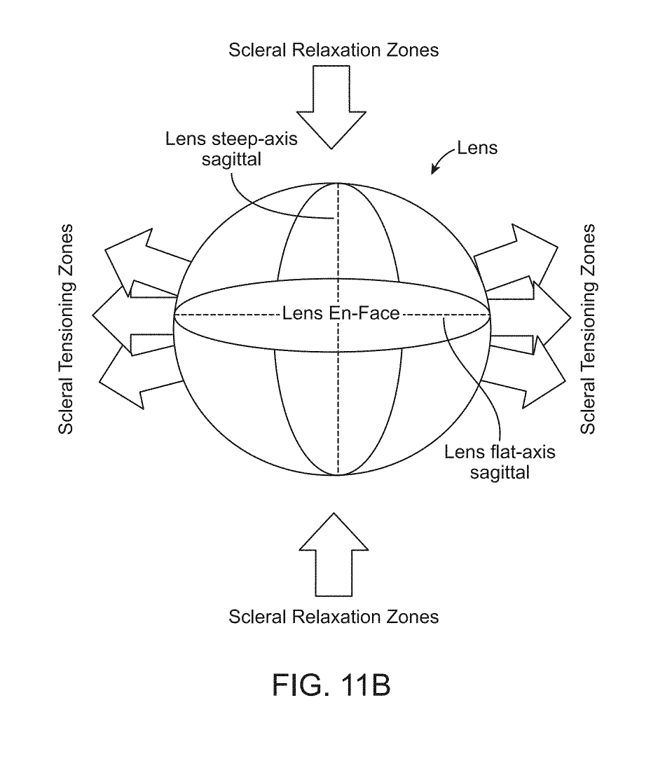

FIGS. 11A1-11A2 show an embodiment of a treatment zone for astigmatism. The HIFU system described herein may be used to induce meridional flattening with 90 degree offset steepening. FIG. 11A1 shows the cornea en-face and FIG. 11A2 shows a cross-section of the cornea including the HIFU system. For example, a bowtie pattern of erosion may be generated by trans-epithelial mechanical erosion of the stromal tissue to effect a bowtie pattern of relative steepening. Alternatively or in combination a bowtie pattern of shrinkage may be generated using the HIFU system in thermal mode. Two HIFU energy beams have been depicted here for simplicity, though it will be understood that the method described here may comprise additional HIFU treatment beams and focal points.

FIG. 11B shows an alternative embodiment of a treatment zone for astigmatism. The HIFU system described herein may be used to generate regional lenticular tensioning by inducing scleral tensioning along the lens flat-axis sagittal. Alternatively or in combination, regional lenticular relaxation may be generation through the induction of scleral relaxation along the lens steep-axis sagittal.

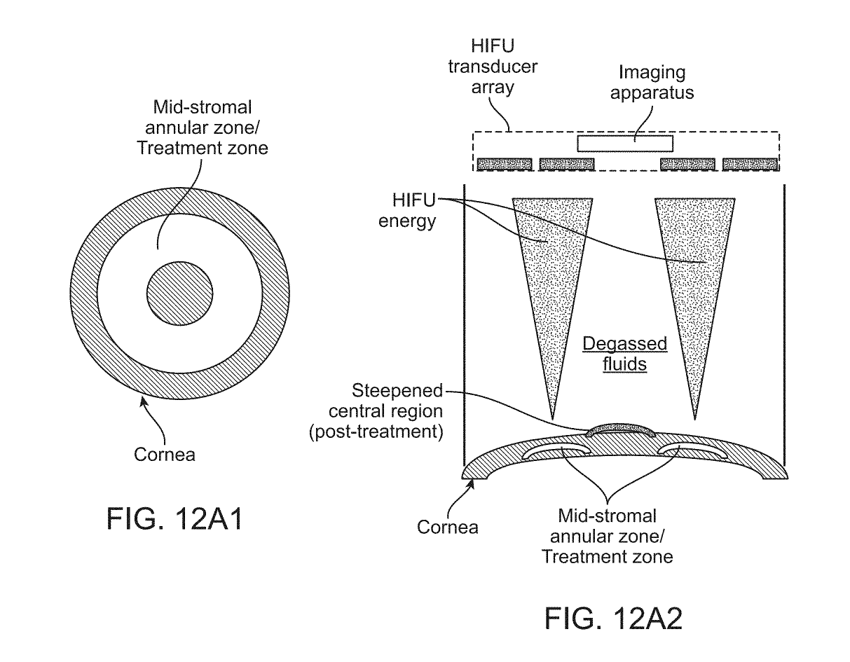

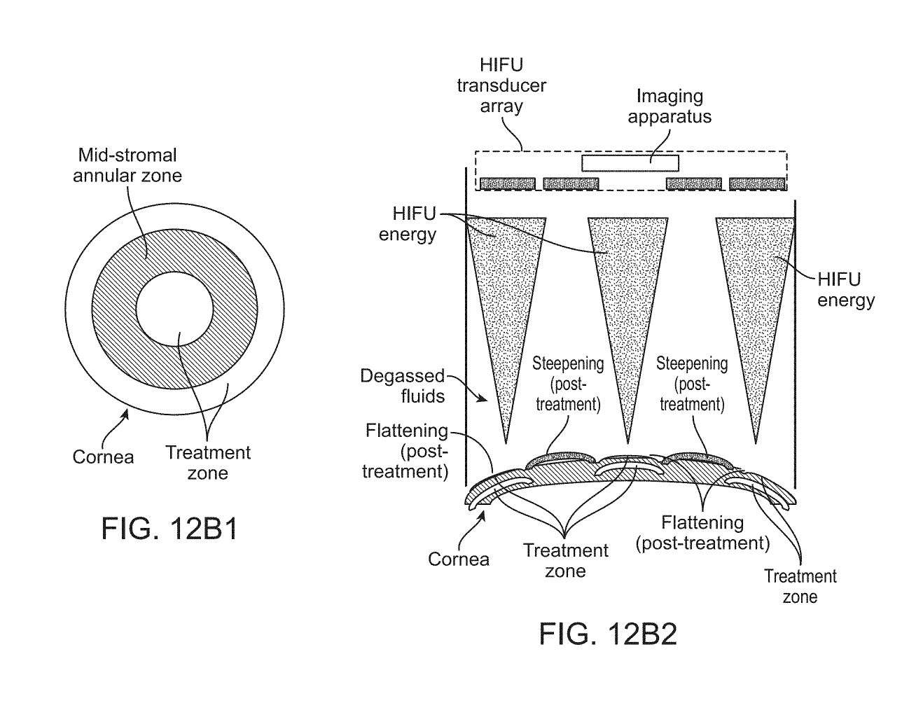

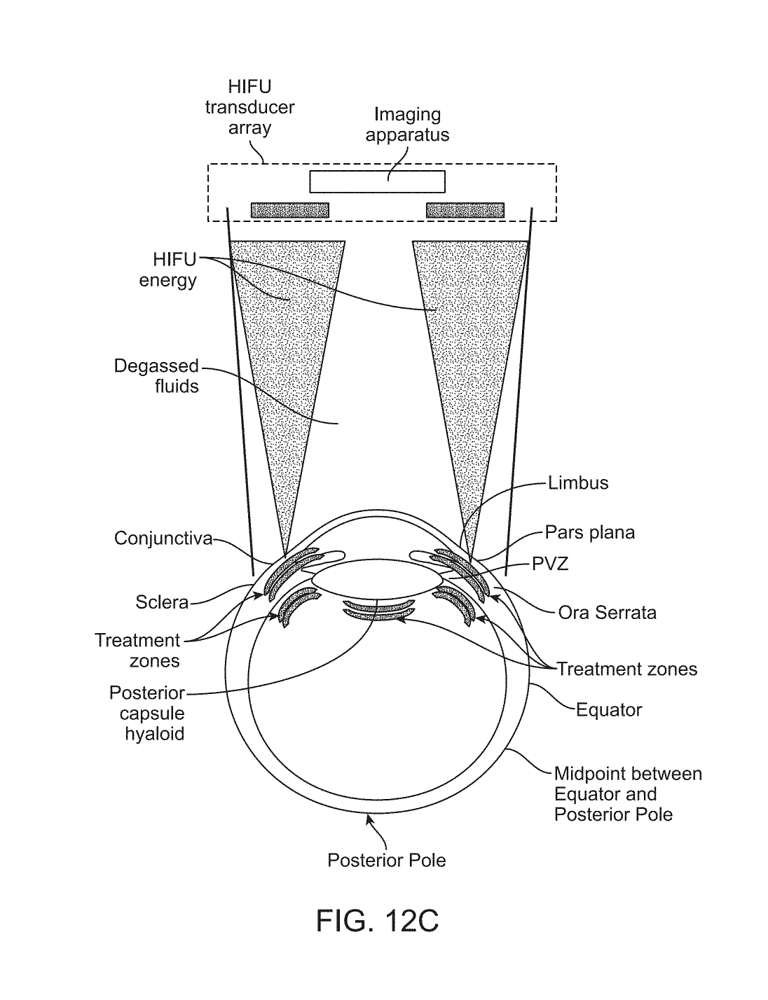

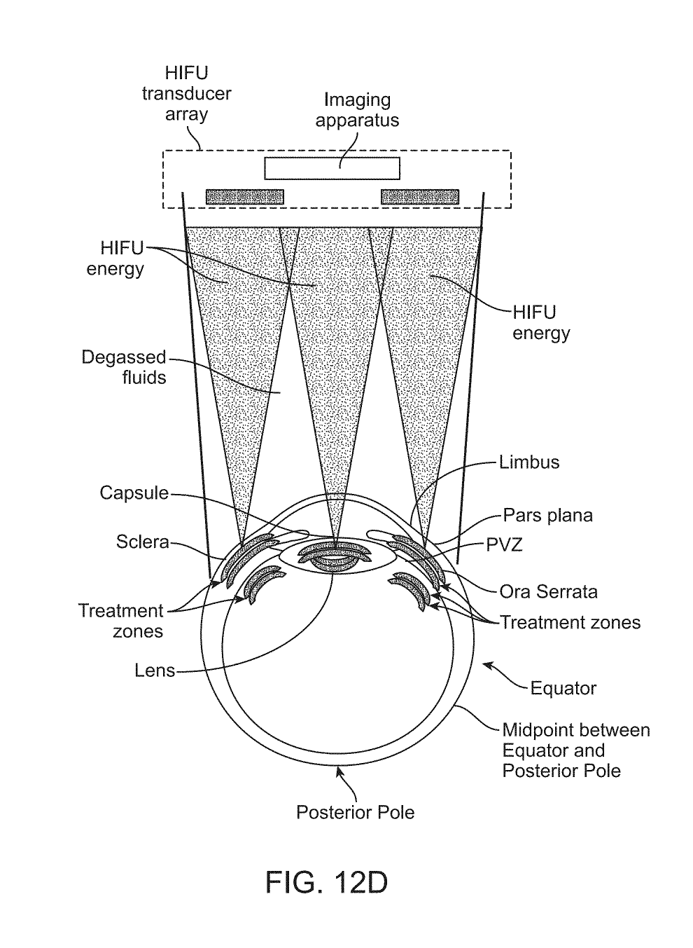

FIGS. 12A1-12A2 show an embodiment of a corneal treatment zone for presbyopia using a center near approach. The HIFU system described herein may be used to steepen the central cornea to relieve center near presbyopia. FIG. 12A1 shows the cornea en-face and FIG. 12A2 shows a cross-section of the cornea including the HIFU system. A mid-stromal annular zone comprising the region about 3 mm to 5 mm from the center of the cornea may be treated trans-epithelially with the HIFU system in mechanical mode to erode tissue and generate a relatively steeper central region of the cornea. Alternatively or in combination, the HIFU system may be operated in thermal mode to generate heat shrinkage in the mid-stromal annular zone. Two HIFU energy beams have been depicted here for simplicity, though it will be understood that the method described here may comprise additional HIFU treatment beams and focal points.

FIGS. 12B1-12B2 show an embodiment of a treatment zone for presbyopia using a center distance approach. The HIFU system described herein may be used to flatten central cornea and peripheral cornea, sparing the mid-stromal annular zone and effectively steepening the cornea in the mid-stromal annular zone. FIG. 12B1 shows the cornea en-face and FIG. 12B2 shows a cross-section of the cornea including the HIFU system. The HIFU system may be operated in mechanical mode to induce annular erosion in the regions surrounding the mid-stromal annular zone. Treatment may be near the surface of the cornea and/or deeper within the cornea. Alternatively or in combination, the HIFU system may be operated in thermal mode to induce heat shrinkage of said regions. Three HIFU energy beams have been depicted here for simplicity, though it will be understood that the method described here may comprise additional HIFU treatment beams and focal points to treat the regions.

The methods described in FIGS. 12A1-12B2 to treat presbyopia may alternatively be used to treat any type of spherical aberration.

FIG. 12C shows an embodiment of a treatment zone for presbyopia using scleral erosion. The HIFU system described herein may be directed sub-conjuctivally to induce scleral erosion at one or more of the pars plana (between the limbus and ora serrata), the PVZ insertion zone, and the PVZ lacunae. Sub-conjunctiva scleral erosion using HIFU may result in softened pars plana, increased circumlental space (CLS), shifting of the PVZ insertion anteriorly, and scaling of the PVZ lacunae. Additionally, delamination of the posterior capsule hyaloid using the HIFU system may further relieve presbyopia and increase accommodation. Two HIFU energy beams have been depicted here for simplicity, though it will be understood that the method described here may comprise additional HIFU treatment beams and scanned focal points to treat the scleral zones.

FIG. 12D shows an embodiment of a treatment zone for presbyopia including lenticular erosion (e.g. partial phacotripsy). The HIFU system described herein may be directed to the lens for sub-capsular erosion and liquefaction of the lens, in addition to the scleral zones described in FIG. 12C, which may result in lens softening and increased accommodation. Three HIFU energy beams have been depicted here at different positions for simplicity, though it will be understood that the method described here may comprise additional HIFU treatment beams and focal points to treat the lens.

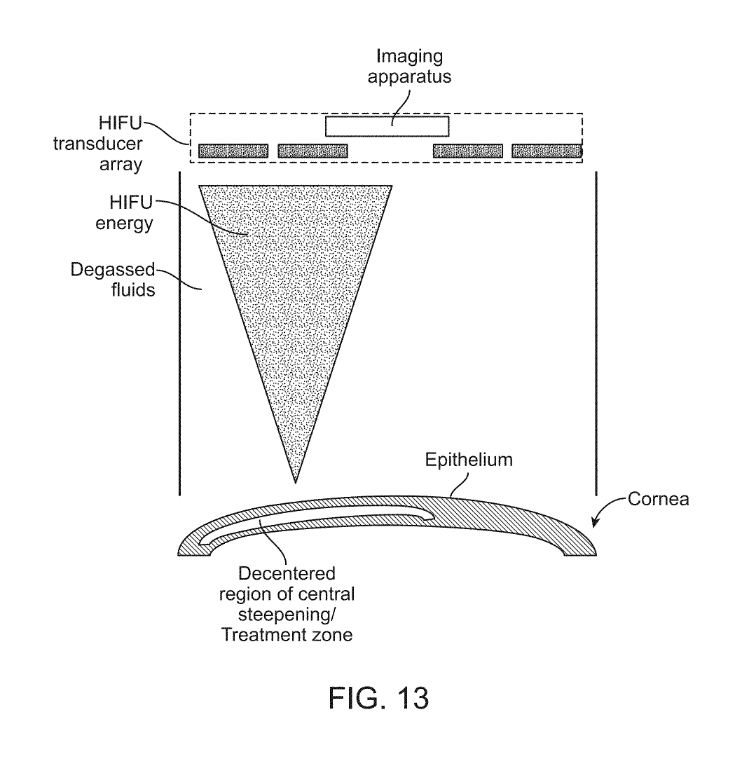

FIG. 13 shows an embodiment of a treatment zone for KCN. The HIFU system described herein may be used to flatten a decentered region of corneal steepening. Using HIFU transducer in thermal mode, HIFU may be directed sub-epithelial into the corneal stroma of the decentered region of corneal steepening in order to thermally shrink said region. The decentered region may be flattened in a dose-dependent manner using topographical imaging to guide treatment. A single HIFU energy beam has been depicted here for simplicity, though it will be understood that the method described here may comprise additional HIFU treatment beams and focal points to treat the decentered region.

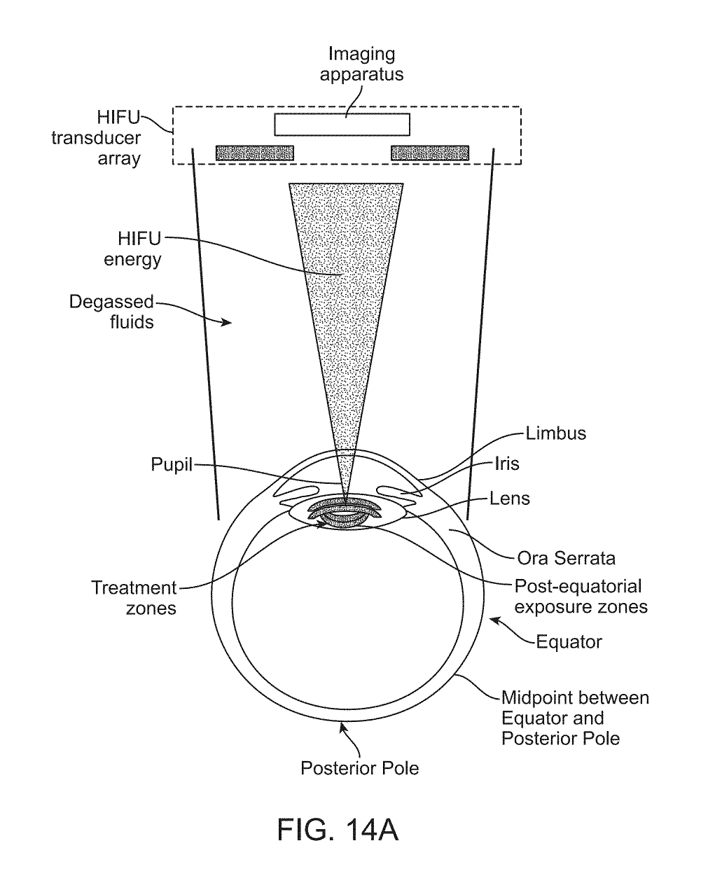

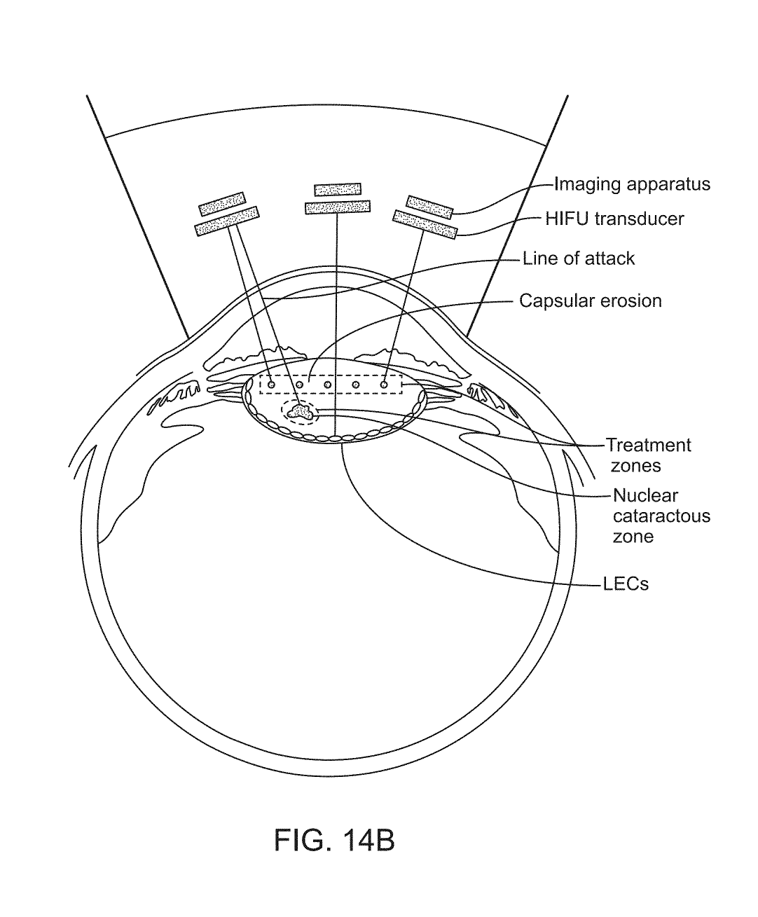

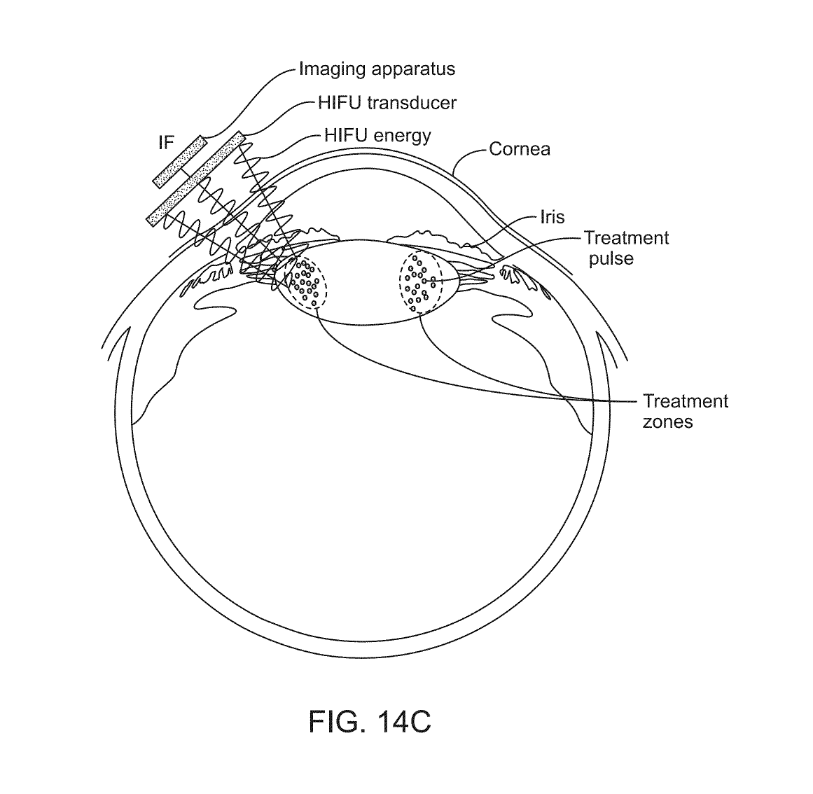

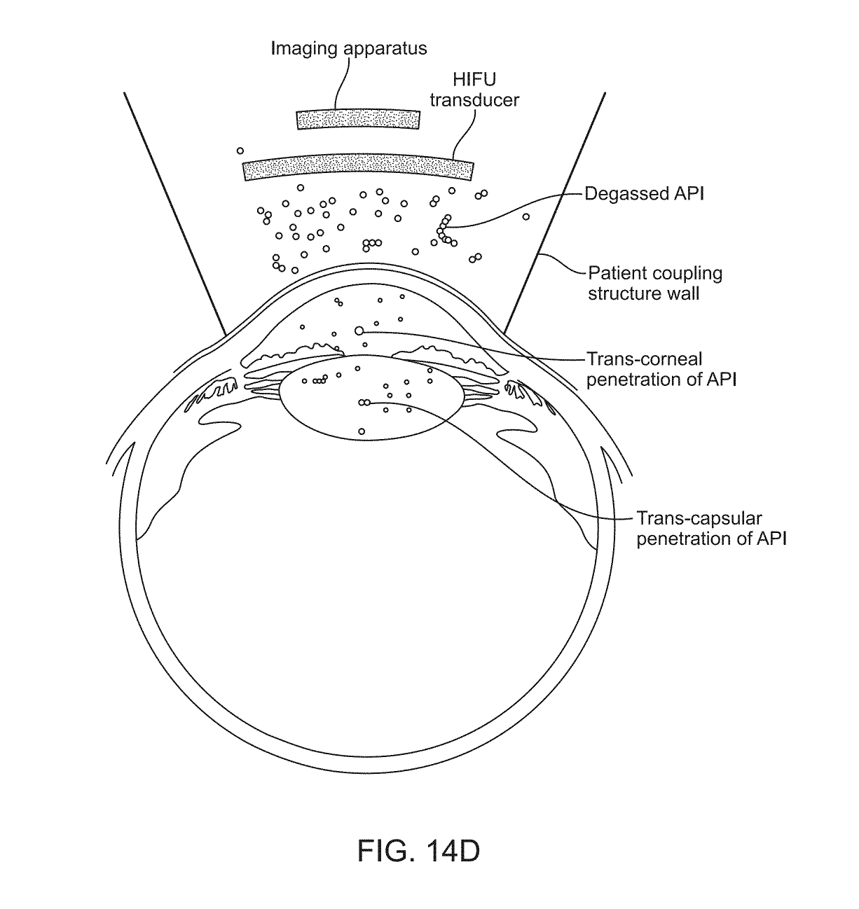

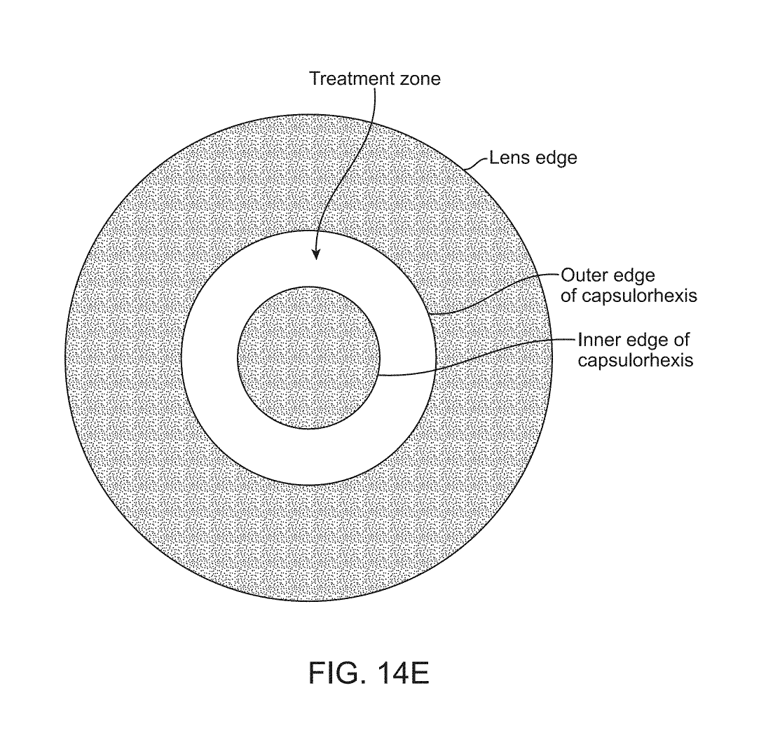

FIGS. 14A-14E show embodiments of a treatment zone for phacoemulsification. FIG. 14A shows an embodiment of a treatment zone for phacoemulsification in which the HIFU transducer may be operated in mechanical mode to induce sub-capsular fractionation and liquefaction to soften the lens. The lens may be bulk softened or partially liquefied or fully liquefied. HIFU energy may be directed trans-pupil and trans-iris. Alternatively or in combination LEC apoptosis may be induced by focusing on posterior capsular and equatorial exposure zones, for example following liquefaction. A single HIFU energy beam has been depicted here for simplicity, though it will be understood that the method described here may comprise additional HIFU treatment beams and focal points to treat the lens.

FIGS. 14B-14E show additional embodiments of a treatment zone for trans-corneal virtual phacotripsy. The HIFU transducer may be focused to the lens in order to decouple the crystalline and fibrillar bonds within the lens to assist in lens removal and replacement or for re-shaping under dis-accommodative or accommodative effort under non-mydriatic conditions. The total capsular volume may decrease proportional to the ultrasound liquefaction volume.

Sclerotripsy may be applied following phacotripsy and post-implant for one or more of adjusting the CLS, adjusting the PVZ insertion, erosion of glistenings, erosion of floaters, treatment of anterior capsular opacification, treatment of posterior capsular opacification, or any combination thereof. Phacotripsy may be applied for non-incisional re-shaping without an implant using one or more of eroding the capsule, softening the lens, softening the cornea, inducing LEC apoptosis, enhancing drug delivery, or any combination thereof.

FIG. 14B depicts multiple transducer elements to indicate possible treatment zones during phacotripsy and do not necessarily represent the number of transducer elements which may be used during treatment, for example to induce capsular erosion. Multiple lines of attack for the therapeutic HIFU arrays have been depicted for example. The HIFU transducers may be phased arrays or discrete arrays. The HIFU transducers include an imaging apparatus for feedback sensing, for example of treatment delivery, tissue elasticity, or temperature as described herein. Treatment with the HIFU system described herein may alternatively or in combination be directed toward LECs” to induce LEC apoptosis and lysis. HIFU treatment may alternatively or in combination be directed to erode a nuclear cataractous zone. FIG. 14C depicts peripheral intra-lenticular sub-capsular treatment zones. The HIFU energy pulses may be directed trans-cornea, trans-iris, or a combination of trans-cornea and trans-iris. FIG. 14D depicts a patient coupling structure comprising a conic-shaped wall defining a fluidic well containing the HIFU transducer and degassed active pharmaceutical ingredient (API). The degassed API may be chilled and may comprise one or more of a chaperone, such as Trehalose, an NSAID, such as aspirin, an anesthetic, such as lidocaine, an anti-inflammatory, an antibiotic, a lens-softener, such as N-acetyl carnosine or Aceclidine, or any combination thereof. The API may penetrate the eye trans-corneal. The API may further penetrate the eye trans-capsular. FIG. 14E depicts a phacotripsy treatment zone for capsulorhexis. The diameter of the lens may be typically about 10 mm. The outer edge of the capsulorhexis zone may be about 5.5 mm. The inner edge of the capsulorhexis zone may be about 5.4 mm. The HIFU system described herein may be focused to the lens in order to erode a ring accurately centered and positioned in Z on the lens by the imaging apparatus.

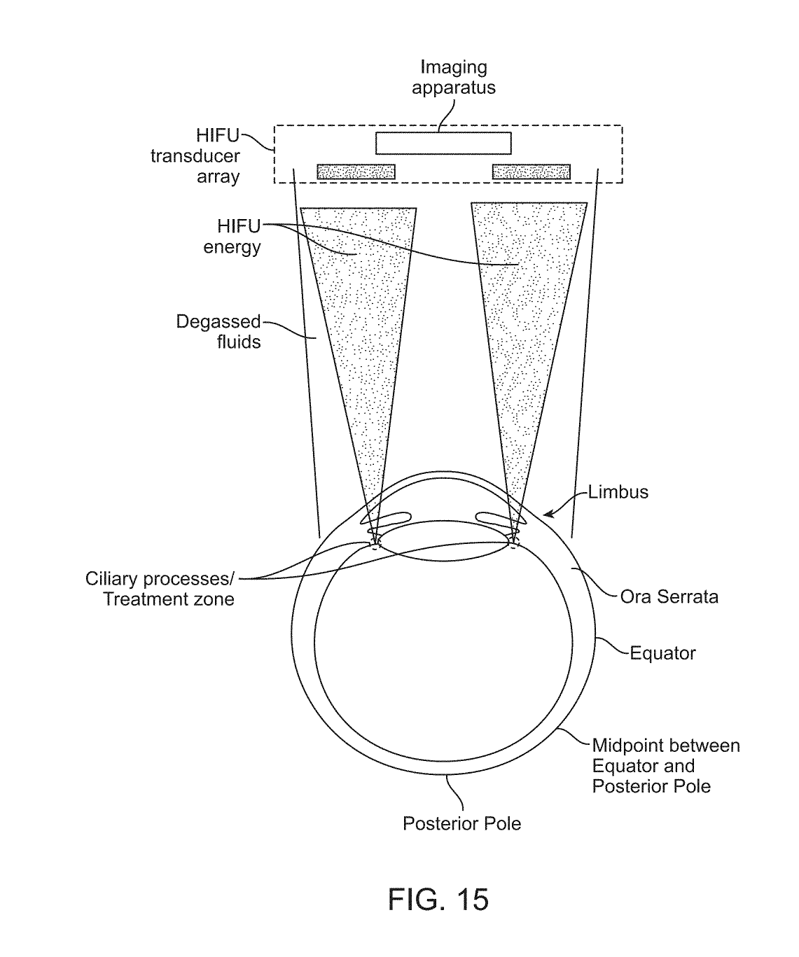

FIG. 15 shows focused pulse locations of an embodiment of a treatment zone for cyclo-sonocoagulation. The HIFU system described herein may be used in thermal mode to direct HIFU energy to the ciliary processes to induce necrosis of cliliary apical cells. Delivery of the HIFU energy may be patterned for 360 degree treatment. Two HIFU energy beams have been depicted here for simplicity, though it will be understood that the method described here may comprise additional HIFU treatment beams and focal points to treat the ciliary processes.

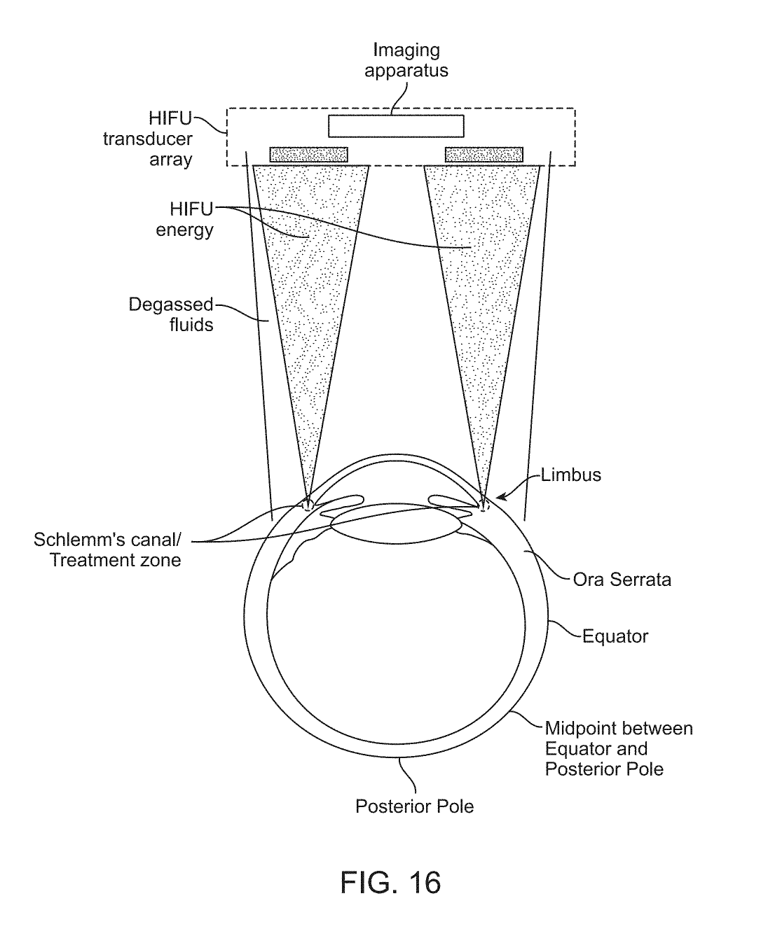

FIG. 16 shows focused pulse locations of an embodiment of a treatment zone for glaucoma. Using the HIFU system in non-thermal mechanical mode, the eye may be treated anywhere on the aqueous outflow path. For example, one or more of the roof of Schlemm's canal and the trabecular meshwork may be fractionated. Alternatively or in combination the canal itself may be treated to remove blockages. Delivery of the HIFU energy may be patterned for 360 degree treatment. Two HIFU energy beams have been depicted here for simplicity, though it will be understood that the method described here may comprise additional HIFU treatment beams and focal points to treat the outflow path.

Alternatively or in combination, the HIFU system may be used in non-thermal mode or thermal mode to emulsify or liquefy a portion of the lens, for example the anterior or posterior surface of the lens, so as to open the angle in a closed-angle glaucomatous eye. The emulsified lens tissue may be naturally worn away or degraded by fluids within the eye over time so as to gradually open the angle and improve aqueous outflow as the lens thickness reduces.

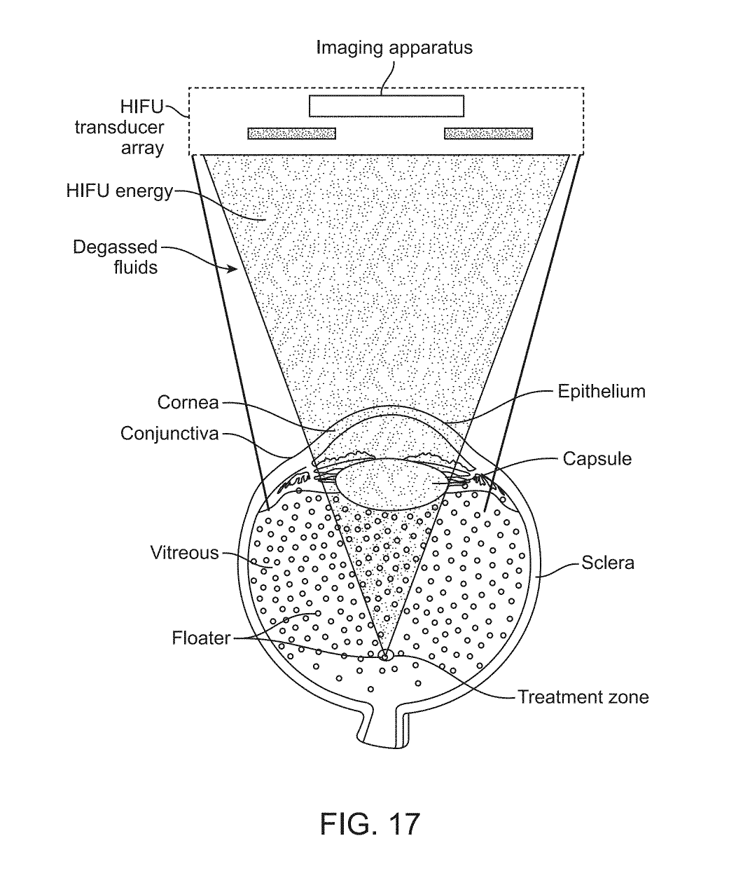

FIG. 17 shows focused pulse locations of an embodiment of a treatment zone for floaters. The HIFU system described herein may be operated in mechanical mode to liquefy or pulverize floaters identified by real-time imaging. A HIFU beam may be focused on the floater trans-pupil, trans-sclera, trans-iris, trans-epithelium, trans-conjunctiva, trans-capsule, trans-cornea, or any combination thereof. The floaters may be acoustically streamed away from the line of site into a non-optically vulnerable region for possible added safety benefits. A single HIFU energy beam has been depicted here for simplicity, though it will be understood that the method described here may comprise additional HIFU treatment beams and focal points to treat one or more floaters.

The methods described in FIG. 17 to treat floaters may alternatively be used for vitreolysis or vitrectomy treatment by focusing the HIFU system to deliver energy to the desired treatment location with the vitreous for liquefaction or spongification of the tissue. Delivery of the HIFU energy may be patterned for 360 degree treatment.

Treatment of the vitreous may alternatively or in combination improve accommodation and be used to treat presbyopia. The peripheral vitreous may stiffen within age, which may impair fluid movement during accommodation and prevent or reduce changes in the shape of the lens. The HIFU system described herein may be used to fractionate (or liquefy or spongify) the vitreous in order to promote fluid transport and enhance shape change of the lens. Selective softening of the vitreous or vitreous structures, for example the peripheral vitreous, may mechanically enhance shape change of the lens independent of or in addition to changes in fluid transport. Softening of vitreous structures may increase the modulus of the vitreous structures.

Cavitation of the vitreous may alternatively or in combination be used to treat myopia, presbyopia, or hyperopia, for example by targeting locations of vitreomacular adhesion (e.g. adhesion between the vitreous and the retina). Detachment of the anterior vitreous from the retina may free the lens from constraint and allow the lens more freedom to move and focus. The HIFU system described herein may be used to cavitationally separate or cut the vitreous from the retina at or near the point of attachment to remove adhesion, thereby reducing the pull of the vitreous on the retina and enhancing lens movement. Anterior vitreal detachment may further be used to prevent macular holes or other damage caused by vitreomacular adhesion known to one of skill in the art. Alternatively or in combination, the vitreous structure may be softened near the point of retinal adhesion so as to locally increase the modulus of the vitreous and reduce or inhibit the effects of adhesion on lens movement.

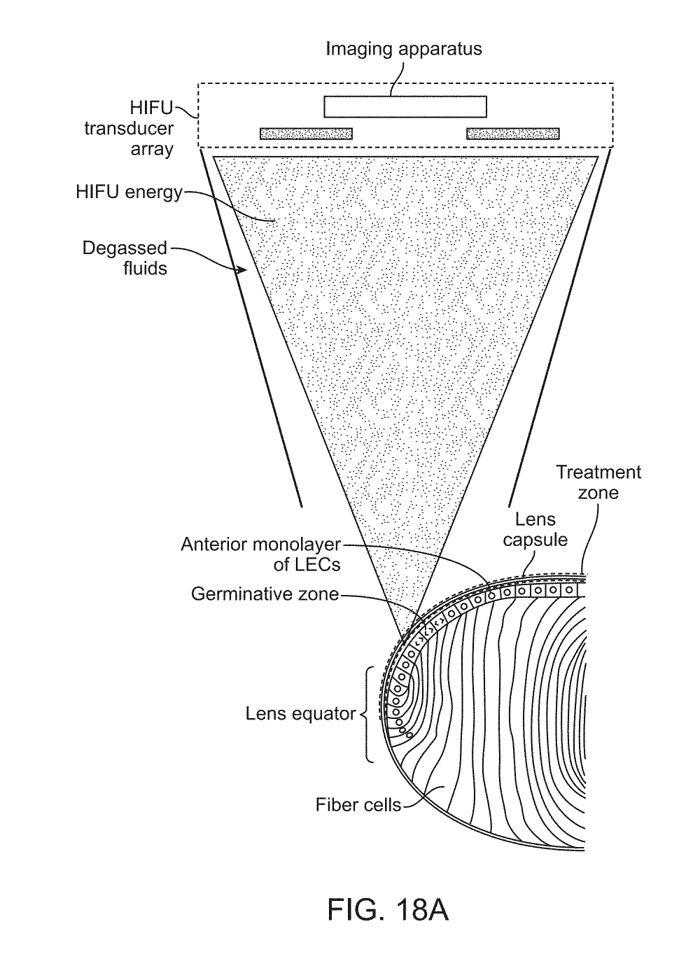

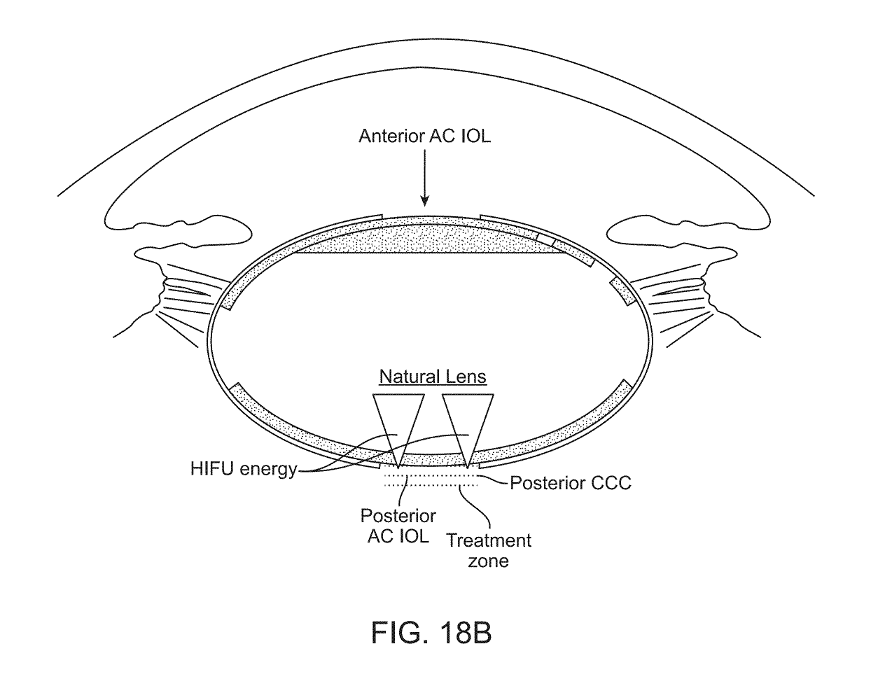

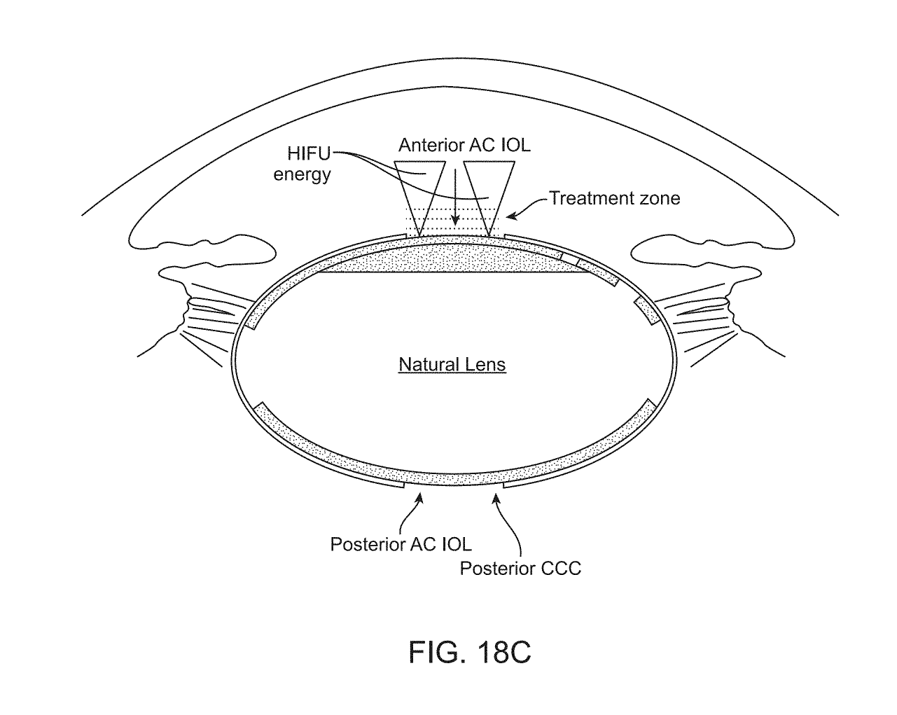

FIG. 18A shows an embodiment of a treatment zone for capsulorhexis. The HIFU system described herein may be used to perform capsulorhexis by focusing the HIFU beam along the lens capsule to induce erosion of the capsule. The HIFU energy may be delivered trans-sclera, trans-iris, trans-pupil, trans-capsular, or by any combination thereof. A single HIFU energy beam has been depicted here for simplicity, though it will be understood that the method described here may comprise additional HIFU treatment beams and focal points to treat the capsule.

FIG. 18B shows an embodiment of a treatment zone for posterior continuous curvilinear capsulotomy (PCCC). The HIFU system described herein may be used to non-thermally induce erosion of the posterior capsule. For example, the diameter of the lens may be measured with an imaging apparatus, for example UBM or OCT, to guide HIFU energy delivery. HIFU energy may be delivered using a circular motion over the posterior lens capsule and cavitation may be monitored real-time with the imaging apparatus. Non-thermal cavitation may for example be induced for about 30 s to erode a region of the lens capsule, for example about 5.25 mm in diameter. PCCC may be pupil-centered or lens-centered.

FIG. 18C shows an embodiment of a treatment zone for anterior continuous curvilinear capsulotomy (ACCC). The HIFU system described herein may be used to non-thermally induce erosion of the anterior capsule. For example, the diameter of the lens may be measured with an imaging apparatus, for example UBM or OCT, to guide HIFU energy delivery. HIFU energy may be delivered using a circular motion over the anterior lens capsule and cavitation may be monitored real-time with the imaging apparatus. Non-thermal cavitation may for example be induced for about 30 s to erode a region of the lens capsule, for example about 5.25 mm in diameter. ACCC may be pupil-centered or lens-centered.

FIG. 19 shows a focused pulse location of an embodiment of a treatment zone for glistenings. Glistenings may occur due to the generation of hydrated or lipid film pockets following insertion of an intraocular lens (IOL). The HIFU system described herein may be used in non-thermal mechanical mode and focused on the disruptions on the IOL identified by imaging. The disruptions may then be eroded by the HIFU system. A single HIFU energy beam has been depicted here for simplicity, though it will be understood that the method described here may comprise additional HIFU treatment beams and focal points to treat one or more film pockets.

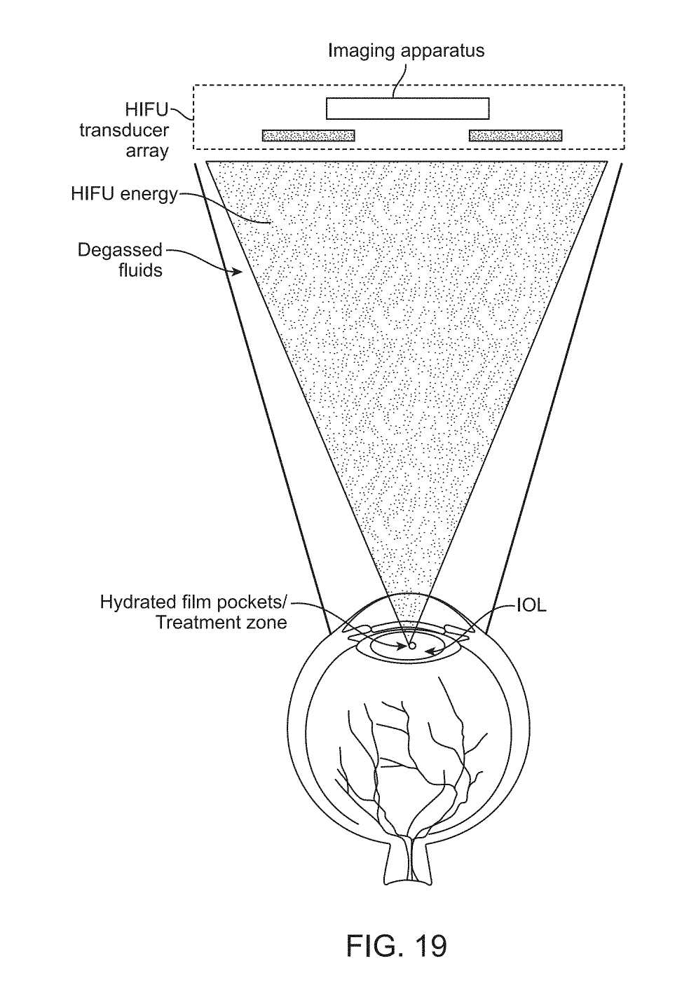

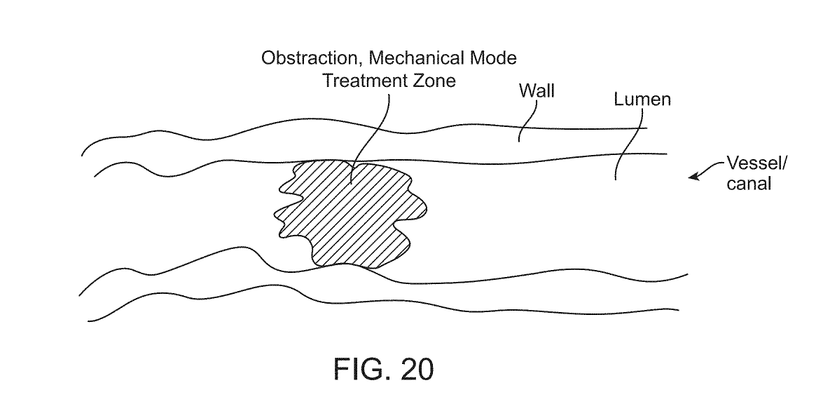

FIG. 20 shows an embodiment of a treatment zone for sonothrombolysis/vascular obstruction. Using the HIFU system described herein, a HIFU energy beam may be directed to treat an obstruction in a vessel or canal of the eye, for example Schlemm's canal. The vessel or canal may comprise a wall and a lumen. Erosion of the obstruction and recanalization may be accomplished by the HIFU system in mechanical mode.

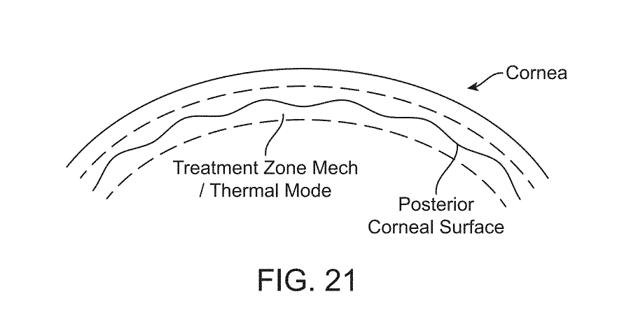

FIG. 21 shows an embodiment of a treatment zone for posterior corneal surface. Using the HIFU system described herein, HIFU energy may be directed to the posterior corneal surface to erode a region of the posterior cornea. The HIFU system may be operated in mechanical mode, thermal mode, or both mechanical and thermal modes to erode tissue. Delivery of the HIFU energy may be patterned for 360 degree treatment. Treatment of the posterior corneal surface may for example be used to treat a cancerous growth.

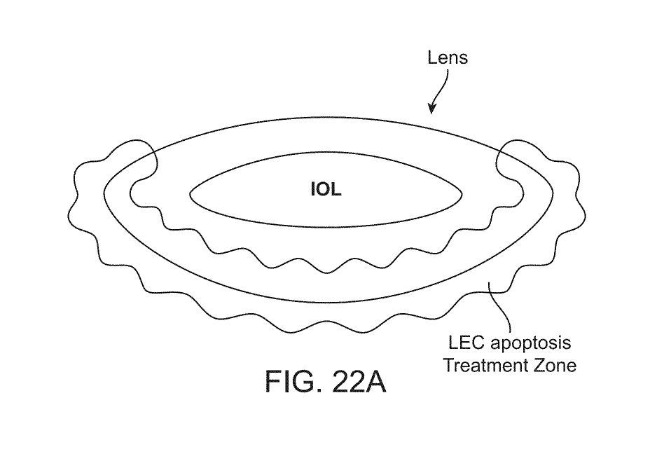

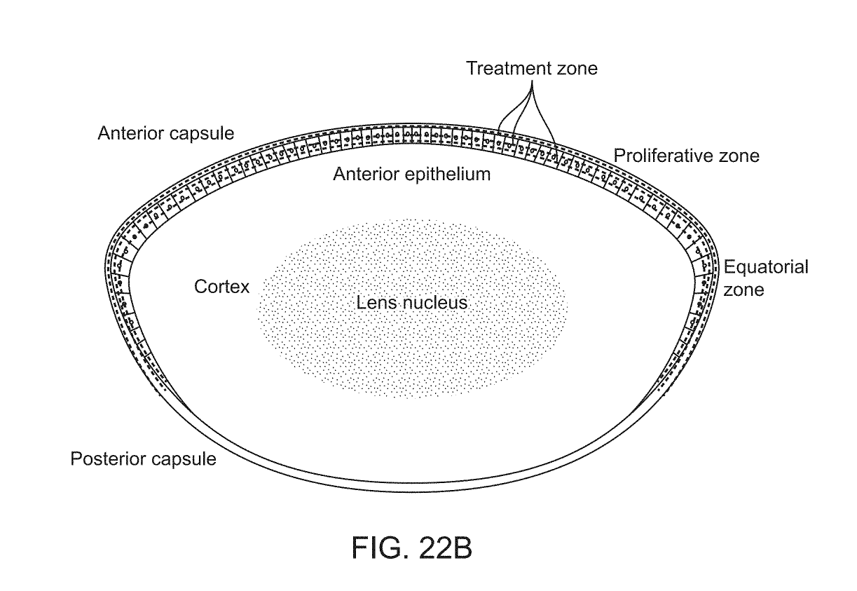

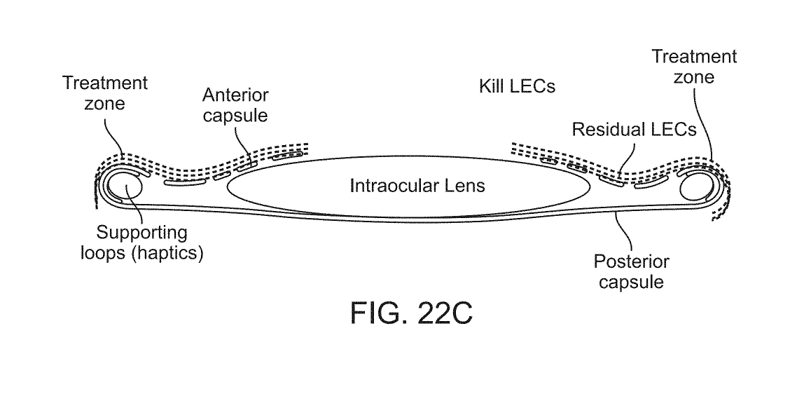

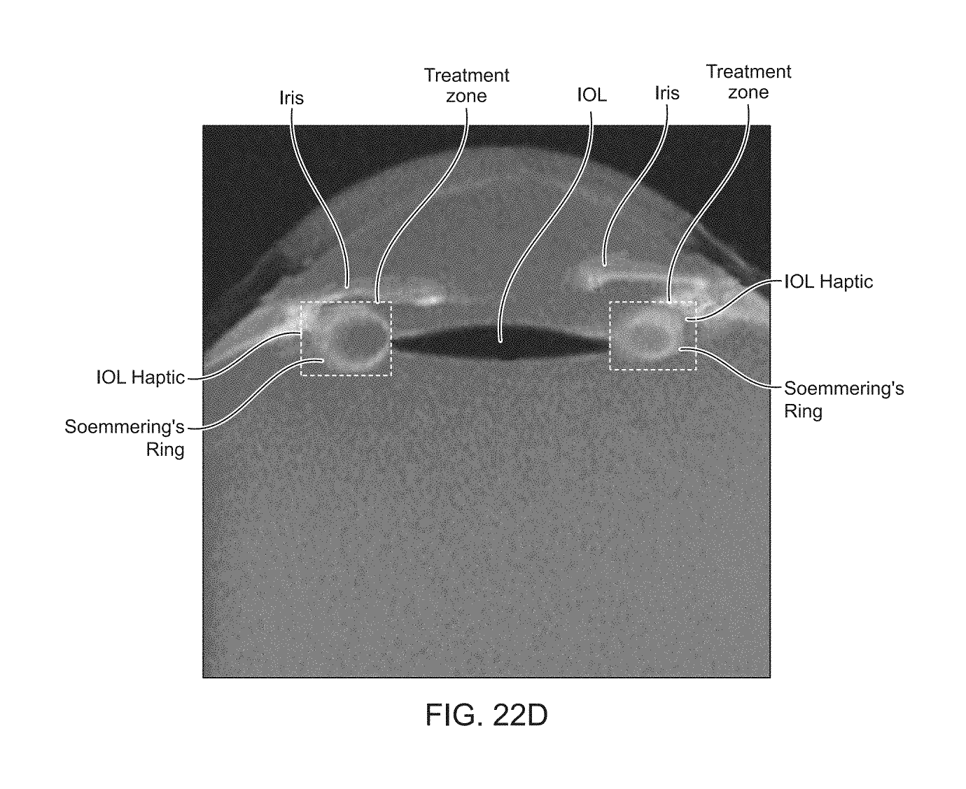

FIG. 22A shows an embodiment of a treatment zone for posterior capsular opacification. Posterior capsular opacification may occur after IOL implantation due to proliferation, migration, or abnormal differentiation of LECs. The HIFU system described herein may be used to induce LEC apoptosis or lysis in order to remove the vision obstructing cells and increase lens clarity. Using the mechanical mode, LECs may be eroded at the focal point of the HIFU beam. This treatment may be repeated at additional focal points. Delivery of the HIFU energy may be patterned for 360 degree treatment. Alternatively or in combination, HIFU energy may be delivered to the anterior capsule in order to induce LEC apoptosis and treat anterior capsular opacification.

FIG. 22B shows an embodiment of a treatment zone for capsule polishing. Capsule polishing may for example be used to treat anterior capsular opacification due to proliferation, migration, or abnormal differentiation of LECs on the anterior capsule. The HIFU system described herein may be used to induce non-thermal HIFU cavitation and LEC apoptosis or lysis along the anterior capsule. Delivery of the HIFU energy may be patterned for 360 degree treatment. Alternatively, delivery of the HIFU energy may be localized to a pre-determined region. Therapy may be guided by an imaging apparatus, for example UBM or OCT. Soemmering's ring and Elschnig's pearls may for example be treated by capsule polishing along the anterior capsule. Additionally or in combination, capsule polishing may be therapeutically beneficial for those diseases along the lens equator.

FIG. 22C shows another embodiment of a treatment zone for capsule polishing. Capsule polishing may alternatively or in combination be used to treat capsular opacification occurring along the lens equator, for example after intraocular lens implantation. The HIFU system described herein may be used to induce non-thermal HIFU cavitation and induce LEC apoptosis or lysis along the lens equator. Delivery of the HIFU energy may be patterned for 360 degree treatment. Therapy may be guided by an imaging apparatus, for example UBM or OCT.

FIG. 22D shows a treatment zone for Soemmering's ring. Soemmering's ring may comprise an annular swelling of the periphery of the lens capsule. This complication may occur following cataract surgery and IOL implantation. As shown in the MRI image of FIG. 22D, Soemmering's ring appears as a hyperintense dumbbell between the IOL and the IOL haptic structure. When viewed face on, the ring is roughly annular or doughnut-shaped around the lens capsule. The HIFU system described herein may be used to induce non-thermal HIFU cavitation to liquefy the fibrotic rings in the treatment zones indicated by dashed lines. Delivery of the HIFU energy may be patterned for 360 degree annular treatment. Therapy may be guided by an imaging apparatus, for example UBM or OCT.

FIG. 22E shows a treatment zone for Elschnig's pearls. Elschnig's pearls are accumulations of pearl-like clusters of proliferative LECs along the posterior lens capsule and may occur following cataract surgery. The HIFU system described herein may be used to induce non-thermal HIFU cavitation to peel the pearls off of the surface of the posterior lens capsule, which may improve vision. Delivery of the HIFU energy may be patterned to include one or more treatment zones comprising an Elschnig's pearl.

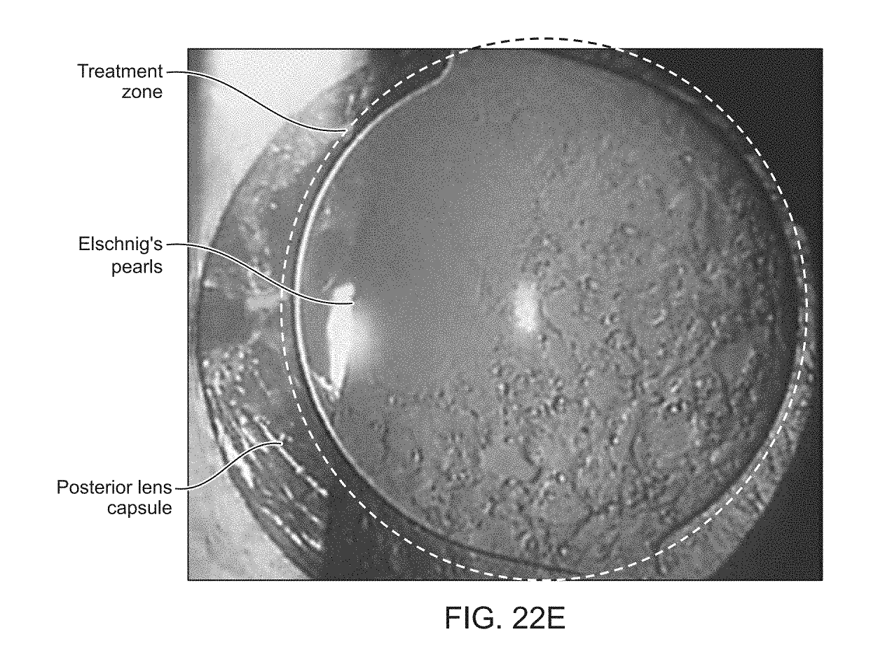

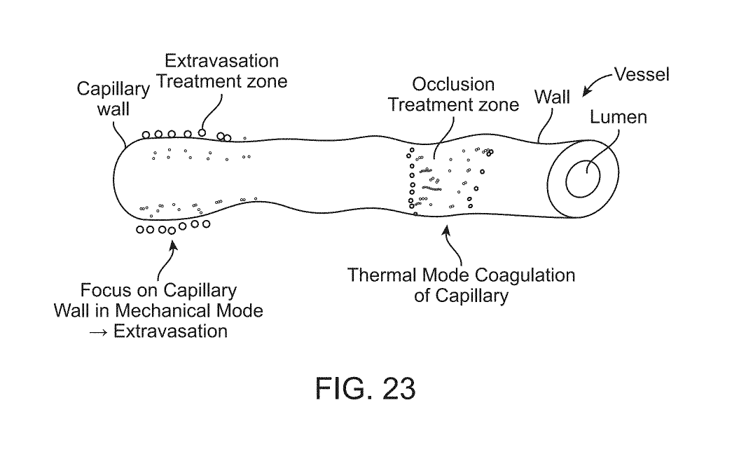

FIG. 23 shows an embodiment of treatment zones for extravasation and occlusion. A vessel may comprise a wall and a lumen. Extravasation or occlusion of a vessel may be induced by focusing HIFU energy to a capillary wall. Using the HIFU system described herein, non-thermal mechanical mode HIFU energy may improve extravasation of the target vessel. Alternatively, the use of thermal mode HIFU energy may induce coagulation of a target capillary and vessel occlusion.

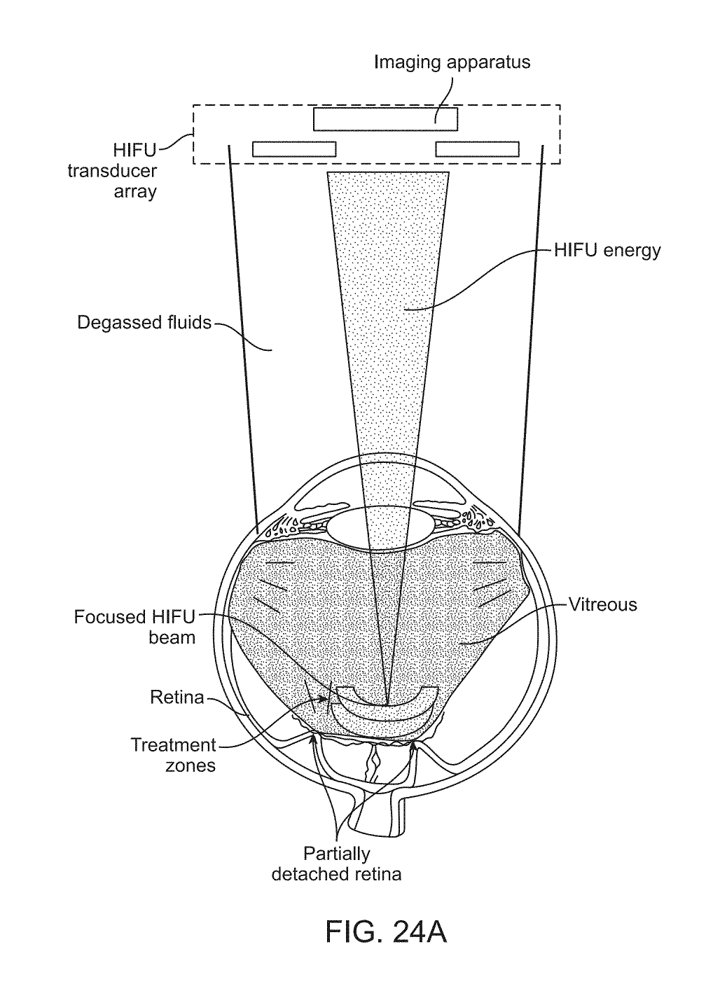

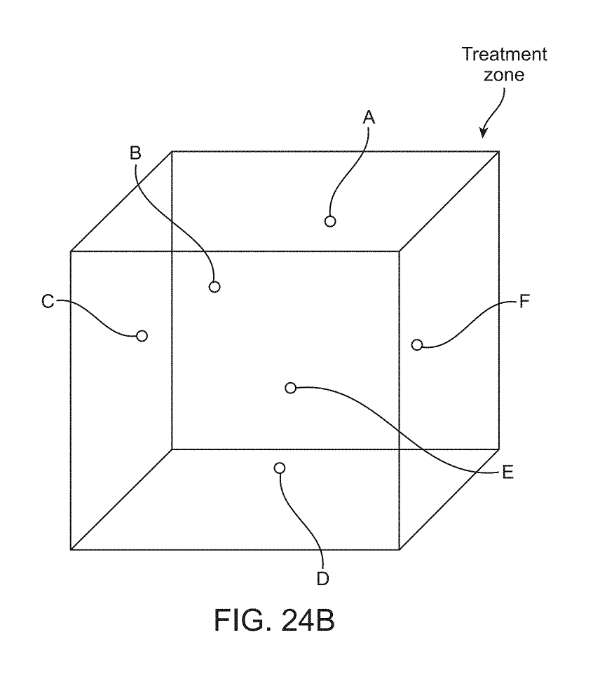

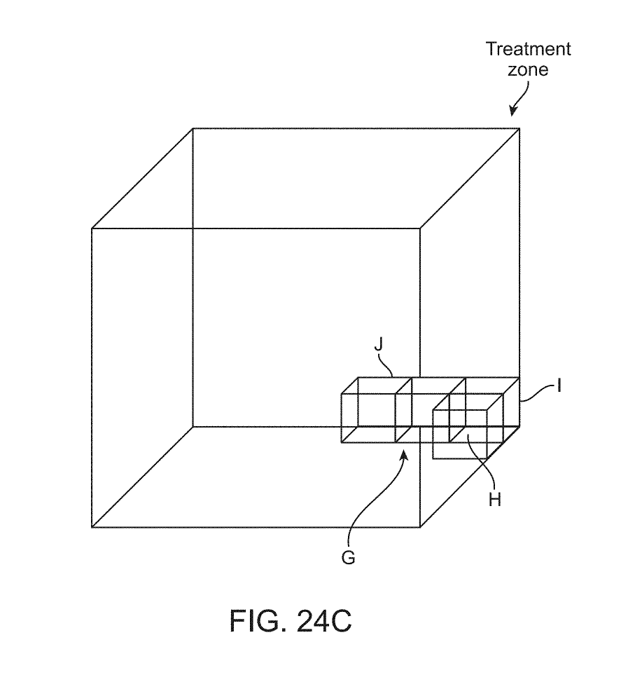

FIG. 24A shows an embodiment of treatment zones for posterior vitreous retinal detachment. Posterior vitreous retinal detachment may occur due to diabetic retinopathy resulting in the vitreous tugging at the retina and partially detaching the retina. Using the system described herein, detachment may be relieved by focusing the ultrasound beam through the ora serrata or the cornea, or both, such that the beam reaches the posterior vitreous body and point of desired detachment. Treatment may be applied to cause delamination and detachment of the retina from the vitreous, thus relieving the detachment.

FIG. 24B shows a tissue treatment zone comprising multiple non-adjacent treatment focal points A-F. Using the HIFU system described herein, the processor may be configured with instructions to sequentially focus on multiple focal points for treatment by a phased array transducer, each treatment focal point A-F being treated non-thermally. The local duty cycle of each treatment focal point A-F may be a non-thermal duty cycle as described herein, for example about 1%. The duty cycle from the phased array may be greater than 50%. Sequential scanning and non-thermal treatment of non-adjacent focal points A-F may allow for the treatment of large volumes of tissue within the treatment zone to be treated very rapidly. Focal points A-F are depicted to represent a possible configuration of treatment points. It will be obvious, however, that the HIFU system described herein may be configured to deliver HIFU therapy to any number of treatment focal points within a treatment zone.

FIG. 24C shows a tissue treatment zone comprising a plurality of adjacent treatment locations of the tissue. The HIFU system described herein may be configured to non-thermally resect the tissue with ultrasound pulses to a plurality of locations of the tissue to define treatment pieces G-J, for example uncut volumetric granular sections of tissue (e.g. voxels), for removal, for example by surgical aspiration. Each of treatment pieces G-J may be defined by a plurality of tissue resection paths, which may for example comprise a plurality of tissue perforations arranged to separate the tissue into the plurality of tissue pieces G-J. The system may be used to non-thermally resect adjacent treatment pieces G-J.

The system described herein may be configured to resect one or more treatment pieces G-J by defining a piece of tissue corresponding to a corrective lens of the eye using ultrasound pulses to a plurality of locations of the tissue. The ultrasound pulses may for example be arranged to allow the piece of tissue to be removed from the eye. The pulses may optionally be arranged to define an access path to the piece of tissue in order to perform a small incision lens extraction (SMILE).

The system may be configured to non-thermally resect the tissue with HIFU energy pulses to a plurality of locations in the tissue to define a 3-D (“three-dimensional”) tissue resection pattern. The HIFU pulses may be configured to cleave collagen fibers during non-thermal tissue resection. The collagen fibers treated may comprise for example collagen fibers of one or more of a cornea, a limbus, a sclera, an iris, a lens capsule, a lens cortex, or zonulae. The pulses may be configured to separate collagen fibers during non-thermal tissue resection.

Such treatment may be used for cataract surgery to remove the lens cortex and insert and IOL into the eye. Alternatively or in combination, 3-D HIFU treatment may be used for capsulorhexis for example.

Voxels G-J are represented herein as cubes, however it will be understood that the treatment pieces G-J may define any 3-D tissue resection or treatment pattern. Treatment pieces G-J are depicted to represent a possible configuration of treatment pieces. It will be obvious, however, that the HIFU system described herein may be configured to deliver HIFU pulses to any number of treatment pieces or locations within a treatment zone. While many of the treatment patterns described herein appear in only one dimension, it will be understood by one skilled in the art that any of the treatment patterns described herein may be 3-D treatment patterns.

In many of the embodiments described herein, one or more HIFU energy treatment beams are depicted for simplicity. It will be obvious however that the methods described herein may comprise additional HIFU treatment beams and focal points beyond those depicted in order to treat the targeted tissue.

It will be apparent to one of ordinary skill in the art that any of the treatment patterns described herein may be scanned sequentially with adjacent and/or overlapping focal points or with non-adjacent focal points.

The HIFU methods and system described herein may additionally be used to treat IK by mechanically inducing regional cellular apoptotic debulking or fibrotic extra-cellular matrix (ECM) debulking or a combination thereof precisely at the infection site. Alternatively or in combination the HIFU system described herein may be used to treat an intraocular tumor. Non-thermal mechanical HIFU may debulk, erode, spongify, liquefy, or debride the tumor to induce apoptosis or necrosis in tumor tissue when directed to the tumor as guided by the imaging apparatus.

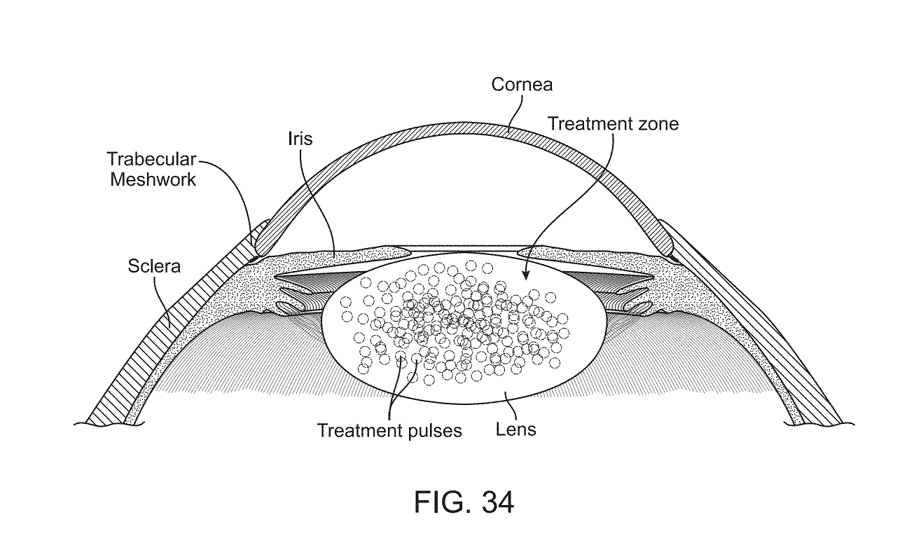

FIG. 34 shows a treatment zone for phacotripsy. The HIFU system described herein may be used to mechanically induce lens softening without affecting the cornea or lens capsule, for example. Treatment pulses may be focused to a treatment zone comprising the lens cortex and nucleus. The treatment zone may be regional, for example the treatment zone may comprise one or more layers of softening at depths of the lens. Softening of the lens may be used to adjust the modulus of the lens from about 50 kPa to about 3 kPa, for example to increase accommodation.

The scanning focused ultrasound beam as described herein can be used to deliver three dimensional treatment patterns to the eye. The processor can be configured with instructions of a computer program in order to treat the eye with the three dimensional scanning beam such as a phased array of a three dimensional scanning beam. The treatment can be planned with imaging, such as OCT imaging for example, and the treatment delivered to the eye in a three dimensional pattern in accordance with the treatment plan.

The focused HIFU beam allows treatment to the eye to be performed at deeper locations of the eye while allowing substantially reduced changes in acoustic pressure near the cornea of the eye. This configuration can have the benefit of treating deeper tissues of the eye while leaving the endothelium of the cornea and epithelium of the cornea and conjunctiva substantially intact, which can improve healing and decrease the invasiveness of the procedure in some instances. The beam can be focused such that the acoustic pressure is approximately inversely proportional to the square of the diameter of the cross section of the beam. For example, the beam can be configured such that the diameter of the beam at the cornea is about 1000 (one thousand) times the area of the beam at the focus, and the corresponding pressure at the cornea is about 0.1% (one tenth of one percent) of the pressure at the focus. The ratio of the ultrasound pressure at the beam focus to the ultrasound pressure at the barrier tissue such as the endothelium or epithelium can be within a range from about 1,000 (one thousand) to about 100,000 (one hundred thousand). For example, with a 10 mm beam passing through the epithelium focused to a 100 um spot, the ratio of the area of the beam cross section at the barrier tissue to the area of the beam cross section at the focus is about 10,000. Based on the teachings provided herein, a person of ordinary skill in the art can increase or decrease size of the beam transmitted through the cornea and the size of the beam at the focus, to provide ratios with therapeutic treatment and decreased damage to sensitive tissues of the eye, such as the tissues of the cornea and retina. The ratio of the areas and corresponding pressures can be within any of the following ranges: from about 1,000 to about 100,000; from about 2,000 to about 50,000; from about 4,000 to about 25,000, for example. Also the ratio of the areas and pressures can be much lower, for example about 100, depending on the application and amount of energy used.

These high numerical aperture treatments allow more accurate treatment with decreased damage to surrounding tissue. Focusing the beam at larger angles results in decreased damage to surrounding tissue. The numerical aperture (hereinafter “N”) of the transducer array can be defined as a maximum dimension across the array (hereinafter “D”) divided by the focal distance (hereinafter “f”), which is the distance from the array to the location where the beam is focused. The numerical aperture of the phased array can be within a range from about 0.5 to about 10, for example within a range from about 0.75 to about 5, for example within a range from about 1 to about 2.5.

The numerical aperture, treatment pressure, and position of the transducer array can be adjusted so as to provide a desired amount of energy to sensitive tissues away from the focused beam while providing treatment with the focused beam.

The system can be configured to provide a first negative acoustic pressure within a first range below a tissue damage threshold at a first tissue location and a second negative acoustic pressure within a second range at a second location to provide the therapeutic treatment. The peak negative acoustic pressure of the HIFU system as described herein may be within a first range at a first location from about 0.001 MPa to about 0.8 MPa and within a second range of about −10 MPa to about −80 MPa at the second tissue location, for example. The first negative acoustic pressure may be within a first range of about −0.02 MPa to about −0.7 MPa and the second negative acoustic pressure may be within a second range of about −20 MPa to about −70 MPa, for example.

The duty cycle of the ultrasound system can be configured in many ways to provide a rapid treatment of tissue. A phased ultrasound transducer array can be configured to provide pulses to several separate locations very quickly. Where a substantially non-thermal effect is desired with a low duty cycle at a treatment location, the transducer array and circuitry can be configured to sequentially provide pulses to a plurality of non-overlapping pulse treatment regions. The non-overlapping pulse treatment regions can be separated by a substantial distance, e.g. one millimeter or more, such that no more than about 10% of the heat energy from one region enters an adjacent region. The phased array transducer can be configured to provide first one or more pulses to a first treatment region with a first duty cycle and a second one or more pulses to a second treatment region; after treatment of the second region with the second one or more pulses, a third plurality of one or more pulses can be applied to the first treatment region and a fourth one or more pulses can be applied to the second treatment region. Additional treatment regions can be defined as is helpful for the treatment. For example, twenty treatment regions can be defined, each having a duty cycle of no more than 5%, and the phased array transducer can be configured to emit pulses with a duty cycle greater than 50%, such that the localized duty cycle to a tissue treatment region can be much lower. For example, the localized duty cycle can be 5% for 20 treatment regions, and the duty cycle of the transducer array can be 100% in a specific example. The number of defined treatment regions can be within a range from about 3 to about 100, and the duty cycle of each region can be within a range from about 1% to about 30% when the transducer array has a duty cycle greater than 50%. Reference is made to FIG. 24C, which shows a plurality of adjacent tissue regions, each of the plurality of adjacent tissue regions can be treated with a duty cycle lower than a duty cycle of the transducer array by sequentially scanning the pulses to each of the plurality of adjacent regions and then scanning the focused beam to each of the plurality of tissue regions. By programming the phase of the phased array transducer the pulses can be directed anywhere within the treatment zone comprising a plurality of tissue regions, and can be directed to the plurality of tissue regions in any order. The system can be configured to cut a plurality of incised objects such as cubes to facilitate movement of the tissue, for example increased tissue plasticity or increased ease of removal with suction. Although reference is made to cubes, the incised objects can have any shape, such as a pyramidal, conical, spherical rhomboid, or tetrahedral, for example. The circuitry coupled to the phased array can be configured with software to direct pulses along the defined surfaces of the incised objects in order to define each of the plurality of objects.

The HIFU system described herein may comprise a HIFU transducer as described herein. The HIFU transducer may comprise any HIFU transducer known to one of skill in the art.

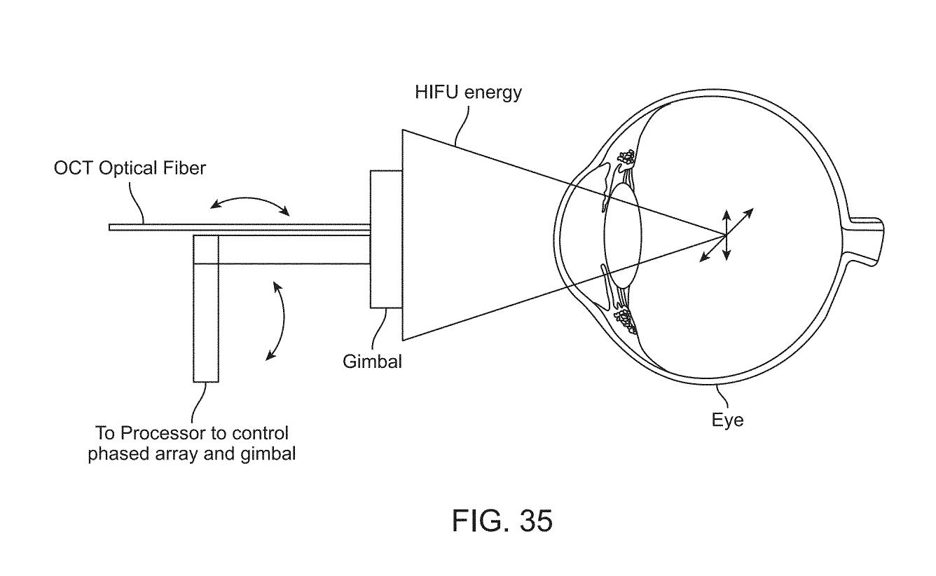

FIG. 35 shows a schematic of an embodiment of a one-dimensional HIFU system. The system may comprise a HIFU transducer array, for example a phased array, coupled to a gimbal for support and movement control. The HIFU array may for example be mounted to an end of the gimbal. The gimbal may provide three degrees of freedom for spot scanning. The gimbal and phased array may be coupled to a processor (not shown) which controls the movement of the gimbal, and thus the phased array and HIFU energy, so as to pattern the HIFU energy beam in a first direction. Alternatively or in combination, the phased array may steer the HIFU energy beam in a second direction transverse or at an angle to the first direction. Thus, the HIFU energy beam may be patterned for treatment using any of the treatment patterns described herein which occur in one or two-dimensions. The HIFU array may be coupled to an imaging system, for example an OCT optical fiber, so as to image the eye before, during, or after treatment in real-time as described herein.

Alternatively or in combination, the HIFU array or gimbal may be mounted to an x-y motorized translation stage which may move the transducer in x, y, or both x and y during treatment. The x-y motorized translation stage may be controlled by a computer or processor as described herein.

Alternatively or in combination, the phased array of the HIFU system may further be configured to provide treatment at depths (e.g. treatments in z) within the tissue. Alternatively or in combination, the HIFU transducer or gimbal may be mounted on an x-y-z translation stage in order to treat tissue at varying depths. The x-y-z translation stage may be under computer or processor control to allow for up to 3-D scanning. Alternatively or in combination, the HIFU transducer may be a 2-D phased array to allow for 3-D volumetric scanning of the tissue.

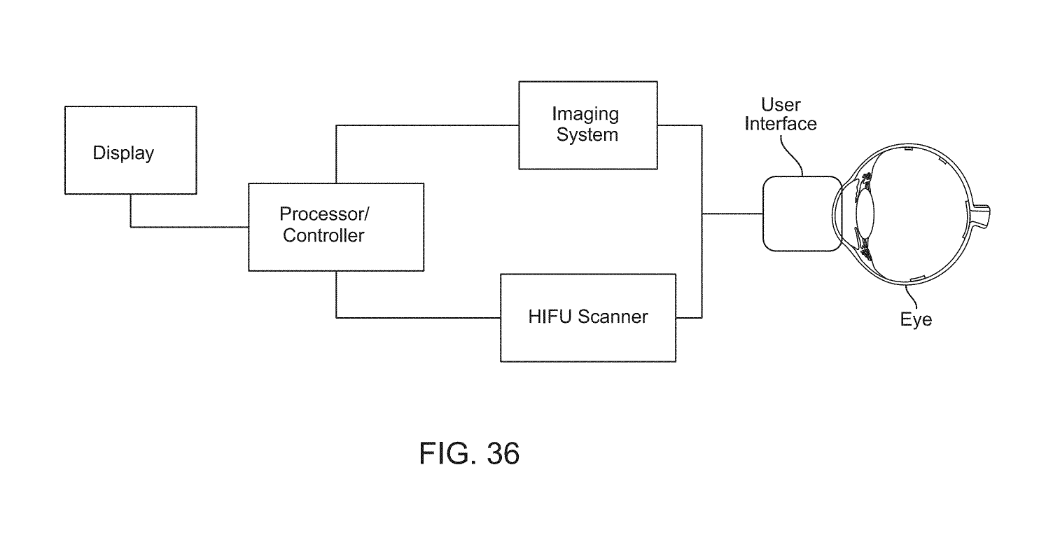

FIG. 36 shows an embodiment of a HIFU treatment system which may be used for any of the treatment methods described herein. The system may comprise a HIFU scanner which directs and scans HIFU energy from a HIFU transducer array to one or more locations on or inside the eye. The HIFU scanner may be coupled to a patient interface or patient coupling structure as described herein. The HIFU scanner may further be coupled to an imaging system, for example OCT or UBM, as described herein. The imaging system may be used to capture one or more images of the eye before, during, or after treatment as described herein. A processor or controller may be coupled to the HIFU array and the imaging system and be configured with instructions to scan the HIFU beam to a plurality of locations and image the tissue during treatment. The system may also comprise a display coupled to the processor that allows the user to visualize the tissue prior to, before, or after treatment. The display may show images which allow the user to see the tissue treated and plan the treatment. Images shown on the display may be provided in real-time and can be used to prior to treatment to allow the user to align the tissue and/or select a treatment zone to target. Identified target treatment zones may be input by the user to program the treatment depth, location, and pattern in response to the images shown on the display. The imaging can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU and to image the eye with an embedded imaging apparatus, for example an OCT probe. The processor coupled to the array can be configured with instructions to provide both ultrasound wavelengths from the array. The imaging apparatus may provide additional tissue feedback data in real-time, for example temperature or elasticity. The system as described herein may comprise an eye tracker as known to one in the art in order to generate real-time images of the eye in order to align or register the target treatment regions of the eye. Pre-treatment images can be measured and registered with real-time images obtained during treatment in order to track the location and orientation of the eye.

The transducer array and the processor may be configured to provide a plurality of pulses to a plurality of separate treatment regions separated by a distance. A duty cycle of each of the plurality of separate treatment regions may comprise a duty cycle less than a duty cycle of the transducer array. The plurality of separate regions may comprise a first treatment region receiving a first plurality of pulses and a second treatment region receiving a second plurality of pulses, wherein the treatment alternates between the first plurality of pulses to the first region and the second plurality of pulses to the second region to decrease a duty cycle of each of the plurality of treatment regions relative to the duty cycle of the transducer array in order to decrease treatment time of the first region and the second region.

The HIFU systems described herein may simultaneously provide imaging guidance, quantitative characterization of the tissue (for example measuring mechanical properties such as elasticity), and perform therapeutic tasks.

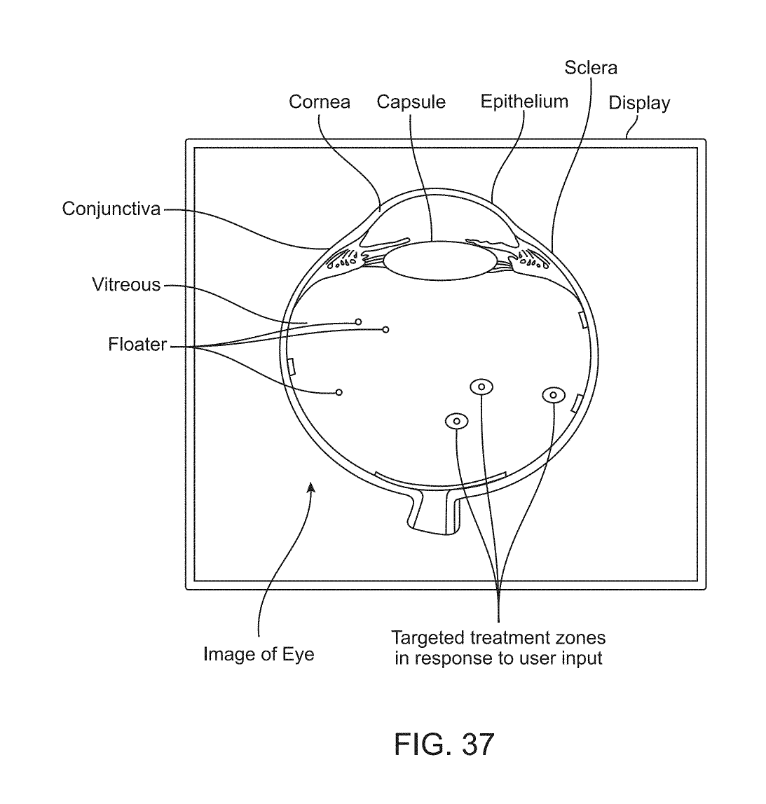

FIG. 37 shows a schematic of a display for use in directing treatment to targeted treatment zones (also referred to herein as targeted treatment regions). The HIFU system described herein may allow for pre-treatment planning and/or treatment of a tissue in an image-guided manner. Treatment locations and patterns may for example be input by a user in response to an image shown on the display. The image may be obtained pre-operatively or in real-time prior to or during treatment. Targeted treatment zones may be selected by a user or operator in response to the image displayed. The user may input the desired treatment zones so as to provide the processor with instructions to scan the HIFU beam to the targeted treatment zones. The user may for example input the desired treatment zones using a touch-screen to select the target zones directly on the displayed image or by using a joystick or mouse to point a cursor at the target zones. For example, the HIFU system may be used to target floaters in the eye. Real-time image(s) of the eye may be acquired and displayed for the user (for example a doctor) to view. The floaters may be identified by the user and selected using a touch screen. The processor may then direct the HIFU scanner to scan the HIFU beam to the targeted treatment zones comprising the floaters. The floaters may be pulverized or liquefied as described herein. While the treatment of floaters is used in this exemplary embodiment, it will be understood by one skilled in the art that a user may input any of the treatment zones or regions described herein in response to the image displayed and the desired treatment.

Image-guided HIFU cavitation may for example be patterned to assist in denervation of a tissue, for example to alleviate pain. Treatment at or near one or more nerves associated with vitreous neovascularization may reduce vitreous rigidity and/or deaden or regress the nerves such that nerve pain is reduced. Sites of inflammation, cancerous lesions, and other localized pathologies may also be targeted using the image-guided HIFU system and methods described herein.

The processor may be configured with instructions to receive user inputs to define the plurality of targeted tissue regions on the image of the eye prior to treatment with the ultrasound pulses. The processor may be configured with instructions to register the plurality of target tissue regions defined prior to treatment with a real time image of the eye acquired during the treatment and to show the target tissue regions of the eye in registration with the real time image of the eye. The imaging system may be aligned with the ultrasound transducer array. The processor may comprise instructions to direct the plurality of pulses to the plurality of treatment regions in response to registration of the real time image of the eye with the image of the eye in response to movement of the eye. The processor may be configured to scan the ultrasound beam to the plurality of locations through an optically non-transparent region of the eye, the region comprising one or more of an iris, a sclera or a limbus of the eye. The imaging system may comprise an ultrasound imaging system and the plurality of treatment regions may be visible on the display and imaged with the ultrasound imaging system through the optically non-transparent region of the eye. The target tissue region may optionally comprise transparent tissue.

The processor may be configured to scan the ultrasound beam to a plurality of locations. The transducer array may comprise a phased array configured to scan the ultrasound beam to the plurality of locations. The system may optionally further comprise an actuator coupled to the ultrasound array to scan the ultrasound beam to the plurality of locations.

The processor and the transducer array may be configured to focus the beam to a plurality of locations in a three dimensional pattern in the eye, The transducer array may be configured to focus the beam to a plurality of different locations along an axis of propagation along the ultrasound beam and/or a plurality of different locations transverse to the ultrasound beam to define a three dimensional treatment region.

The processor may be configured with instructions to generate the HIFU beam comprising a plurality of pulses. Each of the plurality of pulses may comprise at least one acoustic cycle. Each pulse of the plurality of pulses may be separated from a subsequent pulse of the plurality of pulses by a time within a range from about 1 microsecond to about 1000 microseconds in order to provide a duty cycle of no more than about 5 percent (%) to a target tissue region. The plurality of pulses may be arranged to treat a refractive error of the eye, the refractive error comprising one or more of nearsightedness, farsightedness, astigmatism, aberration correction or wave-front aberration correction.

The system may for example comprise a phased array transducer, a one dimensional phased array transducer, a two dimensional phased array transducer, a translation stage, an X-Y translation stage, an actuator, a galvanometer and a gimbal.

The treated pattern may not produce an optically visible artifact to a patient viewing with the eye for a period of time post-treatment within a range from about one week post-treatment to about one month post treatment.

The array and processor may be configured to resect tissue substantially without visible bubble formation. An amount of visible bubbles may comprise no more than 5% of a treatment volume. An amount of visible bubbles may comprise no more than 1% of a resected tissue treatment volume. An amount of visible bubbles comprises no more than 0.1% of a resected tissue treatment volume.

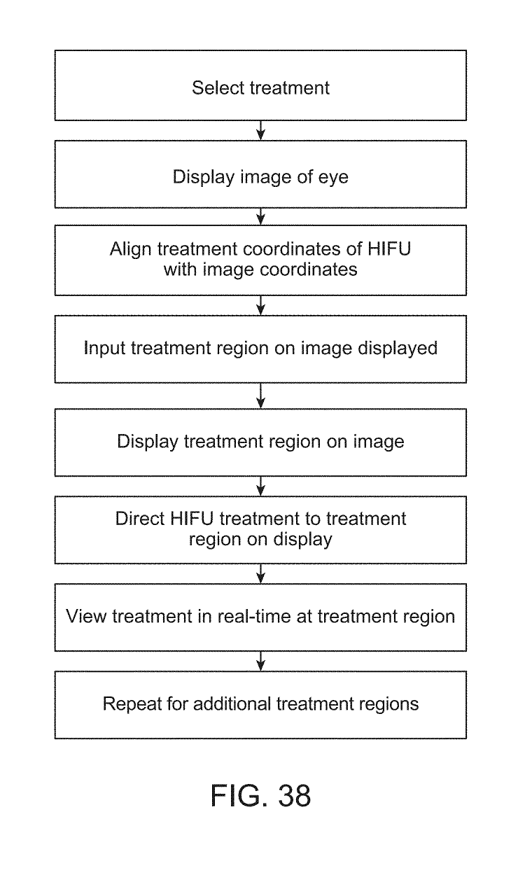

FIG. 38 shows a method for determining a target treatment location. The method may use one or more of the systems described herein. In a first step, a treatment may be selected. Treatment may be directed towards any of the pathologies described herein. In a second step, an image of the eye may be taken and displayed to a user as described herein. In a third step, the treatment coordinates of the HIFU may be aligned or registered with the image coordinates. In a fourth step, the treatment region or zone may be input by a user onto the image shown on the display. In a fifth step, the treatment region may be displayed on the image shown on the display. In a sixth step, HIFU treatment may be directed to the treatment region displayed on the image. In a seventh step, the treatment may be viewed in real-time at the treatment region. In an eighth step, the previous steps may be repeated for additional treatment regions or zones.

Although the steps above show a method of acquiring an image of an eye and treating the tissue at a treatment region selected by a user, one of ordinary skill in the art will recognize many variations based on the teachings described herein. The steps may be completed in a different order. Steps may be added or deleted. Some of the step may comprise sub-steps. Many of the steps may be repeated as often as necessary to treat the tissue as desired.

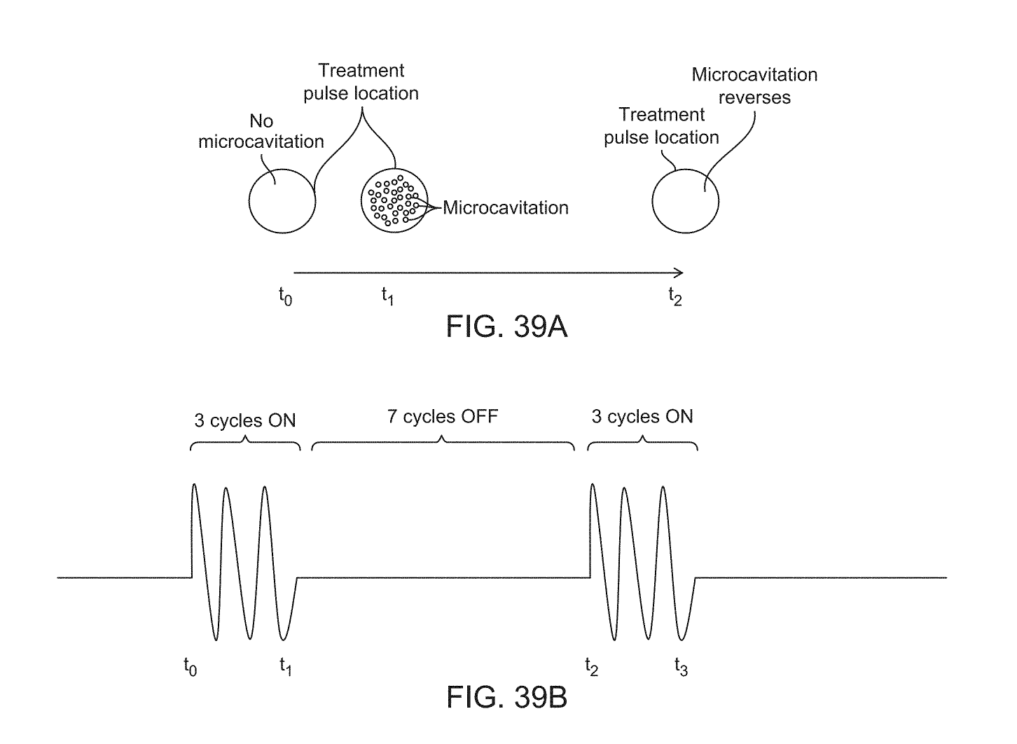

FIGS. 39A and 39B show schematics of the effects of HIFU pulsing over time at a treatment pulse location. Non-thermal cavitation may be temporary, reversible, and/or without bubble formation. During delivery of a HIFU energy pulse to a treatment pulse location, cavitation or microcavitation may occur. Cessation of the HIFU energy may lead to regression of the previously-induced cavitation such that bubbles do not form at the treatment location. Cavitatiton without bubble formation may lead to tissue softening without opacification of the tissue (e.g. the tissue may remain substantially transparent).

The HIFU pulse may comprise a peak negative pressure (or peak negative acoustic pressure) within a range of about −1 MPa to about −80 MPa to generate reversible (non-permanent) cavitation. For example, a HIFU pulse may have a peak negative acoustic pressure within a range of about −1 MPa to about −5 MPa, about −5 MPa to about −10 MPa, about −10 MPa to about −30 MPa, about −30 MPa to about −80 MPa, or within a range between any two numbers therebetween.

FIG. 39A shows the formation and dissipation of microcavitation or cavitation at a treatment pulse location over time. At time t₀, there may be no microcavitation at the treatment pulse location (also referred to herein as the focal point). Between times t₀ and focused HIFU energy may be pulsed so as to generate microcavitation at the treatment pulse location. At time t₁, the HIFU energy pulse may end, leaving a treatment location comprising microcavitation. Between t₁ and t₂, while there is no HIFU energy directed to the treatment pulse location, the microcavitation may dissipate or disappear until it has been partially or completely reversed as shown at time t₂.

FIG. 39B shows an exemplary schematic of HIFU pulsing over time. A treatment pulse may comprise a number of cycles or acoustic oscillations and the pulse may be repeated at a desired duty cycle as described herein. The pulse may for example be “ON” (or pulsing HIFU energy) for 3 cycles or oscillations then “OFF” (or not pulsing HIFU energy) for 7 cycles or oscillations before turning back on for 3 cycles. The frequency of the pulses may thus be 10 cycles with 3 cycles “ON” followed by 7 cycles “OFF” before the next pulse. This pattern may be repeated for the duration of treatment such that the duty cycle of treatment is 30% (that is, each pulse is “ON” for 30% of the time between firing pulses). The duty cycle used to no-thermally induce cavitation may be much lower than in this example. The duty cycle, pulse oscillation rate, and number of cycles “ON” per pulse may be varied as known to one of ordinary skill in the art in order to achieve the desired tissue effects as described herein.

The number of cycles or acoustic oscillations per pulse may be 1, 2, 4, 6, 8, 10, 20, 40, 60, 80, or 100 cycles. The number of cycles may be within a range defined by any two values described herein, for example about 1 to about 10 cycles, or about 1 to about 50 cycles. The number of acoustic cycles may for example be about 1 to about 100 cycles, about 2 to about 50 cycles, about 3 to about 25 cycles, or about 4 to about 12 cycles.

The HIFU system described herein may be used to measure or monitor in vivo elasticity of the lens. The HIFU system may comprise an imaging system capable of performing elastography, for example US elastography, OCT elastography, OCE, or any other elastography imaging system. The imaging system may be used prior to, during, or after treatment to measure the elasticity of a tissue, for example the lens, during treatment to soften or liquefy the tissue. Elastography may be used to inform treatment decisions, for example to titrate the amount of HIFU energy delivered to the tissue and/or determine the treatment region within the tissue in order to reach a target elasticity. For example, a crystalline lens of the eye may be softened or liquefied to reach a target modulus of elasticity in order to improve accommodation and treat presbyopia. The modulus of the lens of a 40 year old eye with presbyopia may for example have a target modulus of less than 50 kPa (for example about 10 kPa to about 50 kPa, or less than about 3 kPa).

The HIFU systems and methods described herein may be used to decrease the modulus of a tissue by at least about 5% without inducing substantial opacification or reducing transparency of the tissue as described herein. The HIFU system and methods described herein may decrease a modulus of the tissue by an amount within a range from about 1% to about 50%. The decrease in modulus of the tissue may remain stable for at least about one week after treatment, for example about one month after treatment or about six months after treatment. Changes in the modulus of the tissue may be measured as known to a person of ordinary skill in the art. For example, OCT, OCE, US cross-correlation functions, time-of-flight US measurements (wherein the speed of sound is dependent on the liquefaction of the tissue and may be mathematically converted into an elasticity value), Brilluoin elastography, or any combination of techniques known to a person of ordinary skill in the art.

The HIFU system and methods described herein may treat the tissue such that the tissue does not opacify and remains optically transparent. The tissue may be treated with the HIFU system in non-thermal mode, thermal mode, or the combination thereof, such that the tissue does not opacify and remains optically transparent. The HIFU system and methods described herein may be used to treat tissue such that change in light scatter as measured by Scheimpflug photometry (e.g. with a Scheimpflug camera) may be within a range of no more than about 5%, for example no more than about 1%. The change in light scatter may for example be within a range of about 0.1% to about 1%, for example about 0.2% to about 1%, or about 0.3% to about 1%. The change in light scatter may be within a range of about 0.4% to about 1%, 0.5% to about 1%, or 0.6% to about 1%. The change in light scatter may be within a range of about 0.07% to about 1%, about 0.08% to about 1%, or about 0.09% to about 1%. The change in light scatter may be within a range of about 0.01% to about 0.09%, about 0.01% to about 0.08%, or about 0.01% to about 0.07%. The change in light scatter may be within a range of about 0.01% to about 0.06%, about 0.01% to about 0.05%, or about 0.01% to about 0.04%. The change in light scatter may be within a range of about 0.01% to about 0.03%, for example about 0.01% to about 0.02%. The change in light scatter may be within a range of about 0.02% to about 0.08%, for example about 0.03% to about 0.07%, about 0.04% to about 0.06%, or about 0.05%.

The HIFU system and methods described herein may treat the tissue such that the tissue does not opacify and remains optically transparent. The tissue may be treated with the HIFU system in non-thermal mode, thermal mode, or the combination thereof, such that the tissue does not opacify and remains optically transparent. The HIFU system and methods described herein may be used to treat tissue such that the change in the index of refraction of the treated tissue is within a range of about 0.01 to about 0.05, about 0.02 to about 0.04, or about 0.03. Light scatter may for example be within a range of about 0.01 to about 0.04, for example about 0.01 to about 0.03, or about 0.01 to about 0.02. Light scatter may for example be within a range of about 0.02 to about 0.05, for example about 0.02 to about 0.04, or about 0.02 to about 0.03.

The HIFU system and methods described herein may treat the tissue with a stability within a range of about 5% to about 25%, for example about 10% to about 20%, or about 15%. The tissue stability may be within a range of about 10% to about 25%, or about 15% to about 20%. The tissue stability may be within a range of about 15% to about 25%, or about 20% to about 25%. The tissue stability may be within a range of about 5% to about 20%, for example about 5% to about 15%, or about 5% to about 10%. The tissue stability may be within a range of about 10% to about 15%.

The HIFU system described herein may be used to enhance drug delivery to the eye. The HIFU system described herein may facilitate targeted drug delivery and/or drug release to a tissue of interest. Drug delivery and/or drug release may be image-guided. The tissue of interest may be imaged before, during, and/or after HIFU treatment to monitor and/or inform treatment decisions regarding drug delivery and/or release. The HIFU system may be operated in mechanical mode to soften or micro-porate any of the tissues described herein in order to enhance porosity of the tissue and improve drug delivery to the treated tissue area. Tissue softening to enhance drug delivery may be performed alone or in combination with any of the methods for treating tissue described herein. Treatment of the tissue of interest may be patterned such that tissue is softened from the site of drug deposition (for example an intraocular/intravitreal injection or artery after systemic delivery) to the target drug treatment site.

For example, cavitational cutting or premeabilization of the vitreous may enhance drug penetration to the retina by increasing the rate of diffusion of the drug through the vitreous. The average pore size of the vitreous is about 6 nm, thus nanoparticles or drugs of about 7 nm or less are typically free to diffuse through the vitreous while larger drugs have decreased rates of diffusion. As such, the vitreous may impede local and systemic delivery of therapeutic drugs to the eye. Cavitation of the vitreous may increase the pore size of the vitreous and enhance drug diffusion and delivery. Alternatively or in combination, cavitation may cause microjets which perturb the local environment of the vitreous around the site of drug deposition and push the drug towards the target drug treatment site. Alternatively or in combination, cavitation may induce fluid velocities, shear forces, and or shock waves in the tissue which may transiently compromise the integrity of cell membranes or tissue and enhance uptake and mobility of the drugs.

Alternatively or in combination, the HIFU system described herein may be used to actively drive the drug into the retina using therapeutic acoustic streaming techniques as known to one of ordinary skill in the art. Acoustic streaming may exert a radiation force which may directionally drive the drug. Radiation force may vibrate components of the vitreous (for example collagen and/or proteoglycans) to produce secondary fluid currents and enhance the apparent diffusion of particles or drugs within the vitreous. Acoustic streaming may be patterned so as to drive the drug from the site of drug deposition in the eye to the target drug treatment site.

Alternatively or in combination, the drug may be conjugated to or encapsulated in a carrier. The carrier may serve to increase the impedance contrast of the drug compared to the surrounding tissue and enhance active delivery of the drug to the target drug treatment site. Alternatively or in combination, the carrier may be sensitive to particular ultrasonic wavelengths such that the drug can be selectively driven through the tissue. For example, the carrier may comprise a particular size and/or structure which enables it to contain the drug while leaving it amenable to receiving ultrasound energy which may drive the carrier-drug complex through the tissue. The carrier may for example be a liposome or other nanoparticle and may be charged or uncharged. The carrier may for example be an intelligent carrier which can be activated and powered by ultrasound, for example micro- or nano-sized “swimmers” or motors which only travel through tissue when sonicated. Such nanoscale motors may move deterministically and be directed to the target drug treatment site using ultrasound (e.g. HIFU). In some instances, the drug itself may be sensitive to ultrasound such that a carrier is not needed for ultrasound-mediated delivery of the drug to the target treatment site.

Alternatively or in combination, the HIFU system described herein may be used for controlled release of a drug at the target drug treatment site. The drug may be encapsulated in a carrier as described herein. The carrier, for example a microcapsule, may be pressure and/or temperature sensitive such that HIFU cavitational treatment triggers release of the drug at the point of sonication, thus allowing for temporal as well as spatial control of drug delivery.

Carriers may include inert gas bubbles, for example argon or other inert gases known to one or ordinary skill in the art, or inert hard or solid nanoparticles, such as gold, aluminum oxide, carbon nanotubes, or others nanoparticles known to one of ordinary skill in the art. Carriers may include liposomes, polymers (for example polyethylene glycol), or other common drug conjugating complexes or nanoparticles.

Alternatively or in combination, the HIFU system described herein may be used to deliver self-assembling therapeutic molecules to a target drug treatment site. Small drug particles may be delivered to the target drug treatment site using any of the methods described herein. The small particles may for example be easily diffused through the tissue or be passively (for example by softening the tissue) or actively driven through the tissue. At the target drug treatment site, the particles may be treated using the HIFU system described herein to induce self-assembly of the particles into larger particles which may enhance drug residence time in the tissue by impairing diffusion away from the target drug treatment site.

The HIFU system described herein may for example be used to deliver nanoparticles for gene therapy of ocular diseases including cataracts, glaucoma, retinitis pigmentosa, age-related macular degeneration, and diseases associated with dystrophies of the photoreceptors. The HIFU system described herein may be used for treatment of any ocular disease as known to one of ordinary skill in the art.

The HIFU system described herein may be used to treat sites of neovascularization of the vitreous for example. Abnormal angiogenesis in the vitreous may be associated with innervation and pain, inflammation, and/or vitreous damage. The HIFU system described herein may for example be used to pattern HIFU cavitational treatment to staunch bleeding from injured vessels. Alternatively or in combination, microbubbles may be injected into the blood stream and targeted in microvessels of interest in order to rupture the vessels and reduce vascularization of the vitreous. Alternatively or in combination, the HIFU system described herein may be used to target anti-angiogenic drug delivery to sites of neovascularization as described herein. The anti-angiogenic drug may for example be encapsulated such that it is able to be locally released by HIFU stimulation at the location of aberrant vascularization and thereby resolve angiogenesis.

Many parameters may be modulated by one of ordinary skill in the art in order to achieve a desired drug delivery or therapeutic outcome including one or more of the ultrasound beam geometry, ultrasound frequency, sonication time, sonication power, drug or particle size, particle shape, particle stiffness or hardness, or the like.

It will be understood by one of skill in the art that the HIFU system and methods described herein may be used to treat one or more clinical pathologies of the eye. For example, treatment patterns may be chosen so as to treat both presbyopia and glaucoma. Any of the treatment patterns described herein may be combined with any number of other treatment patterns to achieve a desired treatment result.

### EXPERIMENTAL

Table 3 describes various HIFU treatment parameters that the inventors have used to induce non-thermal cavitation in pig eyes at various locations in the eye. Locations treated using the methods and system disclosed herein included the corneal surface, sclera, lens, side of lens (lens equator), vitreous fluid, and anterior chamber space. While HIFU frequency was maintained at 1.5 MHz, the pulse-rate frequency (PRF), number of cycles, voltage, and treatment time were varied. Cavitation was induced non-thermally. A PRF of 1000 Hz was used in a majority of the experiments, including Experiments 1-7 and 10-18. Cavitation was able to be induced even when the PRF was dropped to 10 Hz, as in Experiments 19-22.

TABLE 3
Treatment parameters used to non-thermally induce
cavitation at various locations in the eye.

| | | US Fre- | | | Duty | Volt- | Total |
|---|---|---|---|---|---|---|---|
| | Treatment | quency | PRF | No. | cycle | age | Time |
| Exp. | Site | (MHz) | (Hz) | Cycles | (%) | (V) | (h:mm:ss) |
| 1 | Corneal | 1.5 | 1000 | 10 | 0.67 | 200 | 0:02:47 |
| | Surface | | | | | | |
| 2 | Corneal | 1.5 | 1000 | 10 | 0.67 | 200 | 0:02:07 |
| | Surface | | | | | | |
| 3 | Corneal | 1.5 | 1000 | 10 | 0.67 | 200 | 0:06:15 |
| | Surface | | | | | | |
| 4 | Sclera | 1.5 | 1000 | 10 | 0.67 | 200 | 0:04:55 |
| 5 | Sclera | 1.5 | 1000 | 10 | 0.67 | 200 | 0:05:05 |
| 6 | Sclera | 1.5 | 1000 | 10 | 0.67 | 200 | 0:05:00 |
| 7 | Lens | 1.5 | 1000 | 10 | 0.67 | 200 | 0:10:00 |
| 8 | Lens | 1.5 | 200 | 10 | 0.13 | 200 | 0:10:00 |
| 9 | Lens | 1.5 | 200 | 10 | 0.13 | 200 | 0:10:00 |
| 10 | Side of Lens | 1.5 | 1000 | 10 | 0.67 | 250 | 0:08:00 |
| 11 | Side of Lens | 1.5 | 1000 | 10 | 0.67 | 250 | 0:05:00 |
| 12 | Side of Lens | 1.5 | 1000 | 10 | 0.67 | 250 | 0:05:00 |
| 13 | Vitreous | 1.5 | 1000 | 10 | 0.67 | 250 | 0:05:00 |
| | Fluid | | | | | | |
| 14 | Vitreous | 1.5 | 1000 | 10 | 0.67 | 250 | 0:07:00 |
| | Fluid | | | | | | |
| 15 | Vitreous | 1.5 | 1000 | 10 | 0.67 | 250 | 0:05:00 |
| | Fluid | | | | | | |
| 16 | Anterior | 1.5 | 1000 | 10 | 0.67 | 250 | 0:05:00 |
| | Chamber | | | | | | |
| 17 | Anterior | 1.5 | 1000 | 10 | 0.67 | 250 | 0:05:00 |
| | Chamber | | | | | | |
| 18 | Anterior | 1.5 | 1000 | 10 | 0.67 | 250 | 0:05:00 |
| | Chamber | | | | | | |
| 19 | Anterior | 1.5 | 10 | 50 | 0.03 | 250 | 0:05:00 |
| | Chamber | | | | | | |
| 20 | Anterior | 1.5 | 10 | 50 | 0.03 | 250 | 0:05:00 |
| | Chamber | | | | | | |
| 21 | Anterior | 1.5 | 10 | 50 | 0.03 | 250 | 0:05:00 |
| | Chamber | | | | | | |
| 22 | Sclera | 1.5 | 10 | 50 | 0.03 | 250 | 0:03:00 |

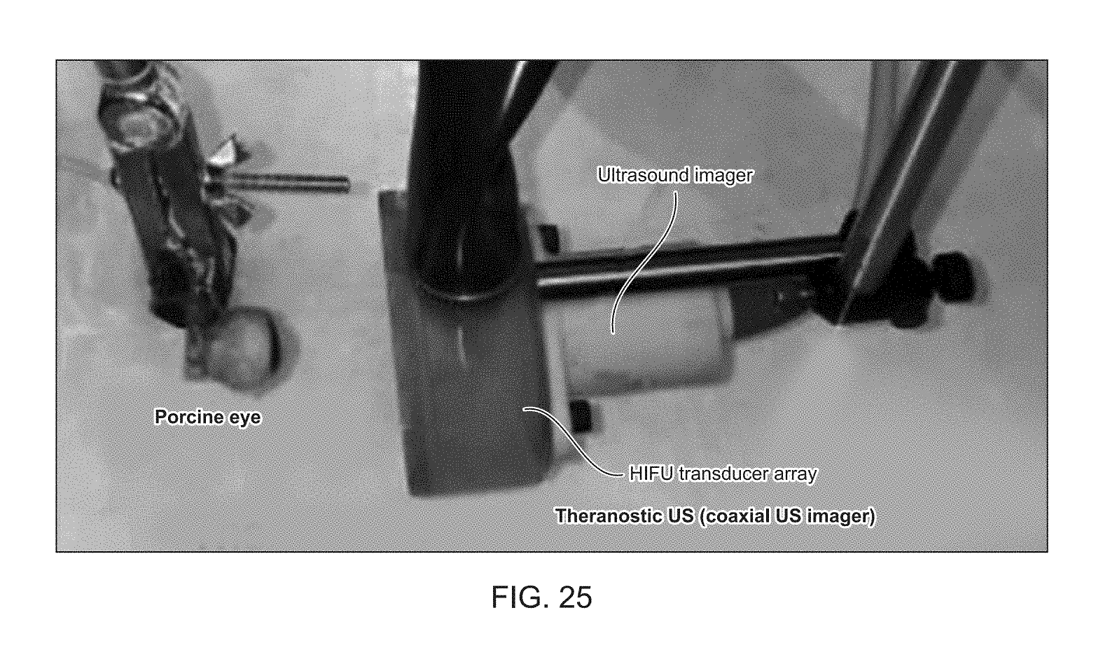

FIG. 25 shows the experimental setup utilized to generate the data presented in Table 3. The HIFU system used to perform Experiments 1-22 (e.g. theranostic ultrasound system) comprised a HIFU transducer array with a central channel in which a coaxial ultrasound imager was disposed. The HIFU transducer was arranged to focus on the treatment area described for each experiment. The imager was used to monitor the HIFU treatment for cavitation. Each experimental eye was kept chilled and temperature changes were monitored. The system further comprised an amplifier, imaging engine, OCT imaging probe, and positioning element.

The HIFU transducer array used to perform Experiments 1-22 was a focused array manufactured by Sonic Concepts, Inc. It will be apparent that any HIFU transducer array known to one of ordinary skill in the art may be used. The ultrasound imager used to perform Experiments 1-22 was manufactured by Ultrasonix. It will be apparent that any imager (ultrasound, OCT, MR, or other as described herein) may be used. It will be apparent to one of ordinary skill in the art that any combination of HIFU transducer and imager as may be used.

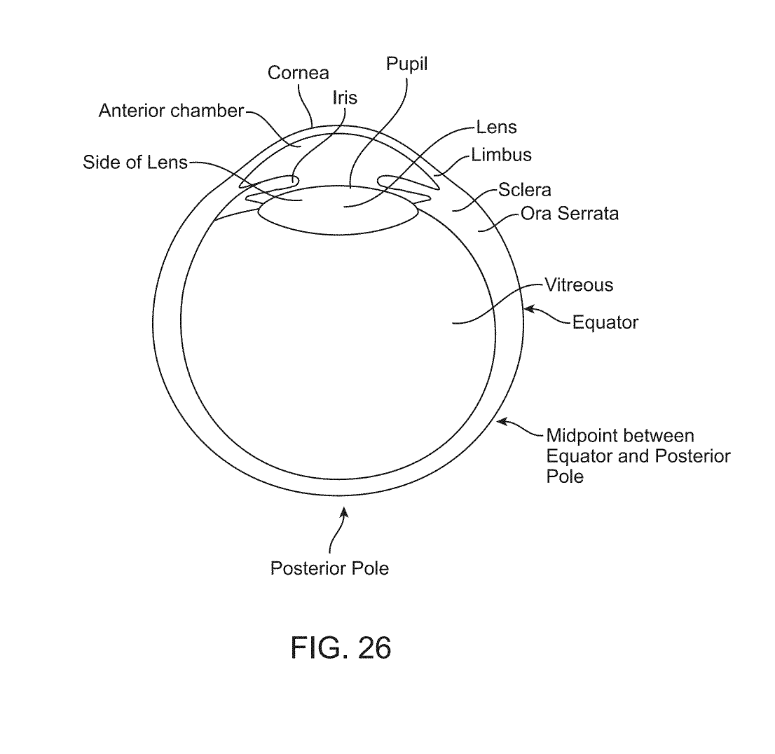

FIG. 26 shows treatment site locations described in Table 3. The HIFU system was focused for each experiment on one of the treatment sites identified in the figure as described in Table 3. Experiments 1-3 focused on the cornea. Experiments 4-6 and 22 focused on the sclera. Experiments 7-9 focused on the lens through the pupil, while Experiments 10-12 focused on the side of the lens through the iris. Experiments 13-15 focused on the vitreous through the sclera. Experiments 16-21 focused on the anterior chamber through the cornea.

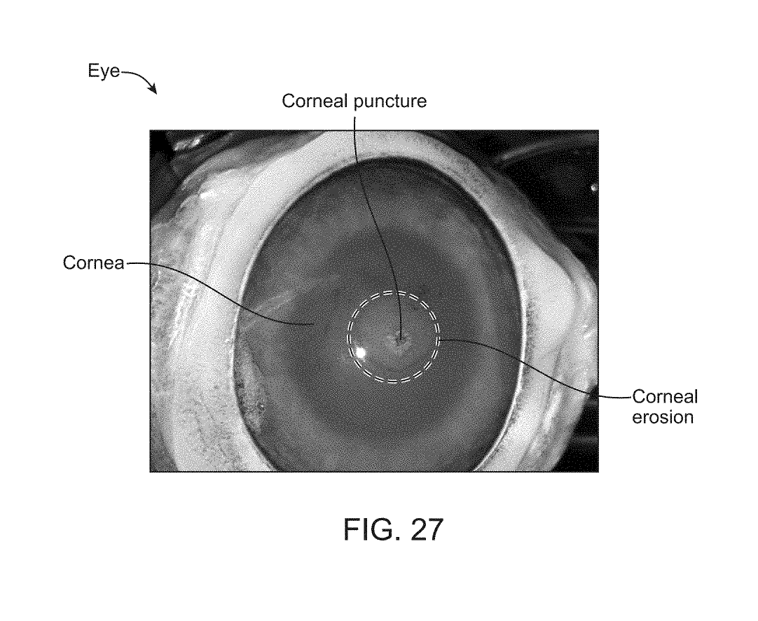

FIG. 27 shows the results of Experiment 1. Using the HIFU parameters described in Table 3, non-thermal cavitation was induced in the corneal surface to generate a central corneal puncture of about 2 mm in diameter within a central region of corneal erosion, thus demonstrating the ability of the methods and system disclosed herein to erode tissue in a non-thermal manner. Such erosion may have therapeutic applications, for example the creation of a cataract incision for phacoemulsification or erosion of a tumor or infectious lesion.

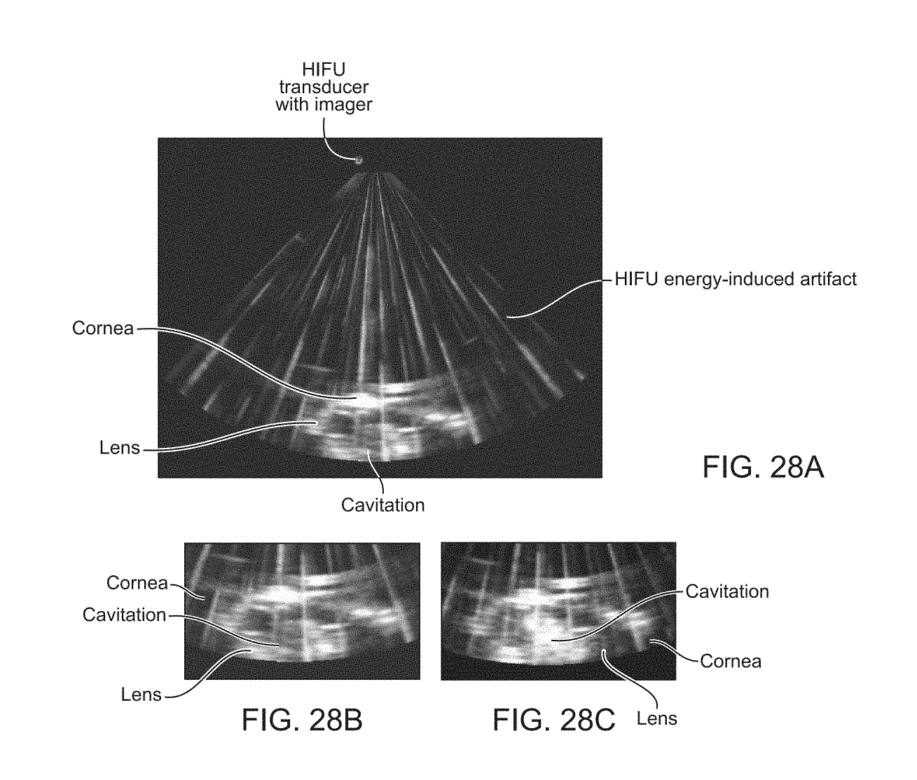

FIGS. 28A-28C show the results of Experiment 10. Using the HIFU parameters described in Table 3, non-thermal cavitation was induced in the side of the crystalline lens using HIFU delivered trans-cornea and trans-iris. FIG. 28A shows an ultrasound image taken by the ultrasound imaging system used to monitor the effects of HIFU therapy. The transducer was located above the eye and HIFU energy beams were focused on the eye below such that the beams were directed towards the side of the lens through the cornea. HIFU energy beam-induced artifacts may be visible on the display as shown when using as high-speed ultrasound imaging system (e.g. a UBM system with a frame rate of 50 to 100 Hz, for example 60 Hz) due to field perturbation by the low duty cycle HIFU. FIG. 28B shows an ultrasound image of the eye during HIFU after cavitation began to occur. FIG. 28C shows an ultrasound image of the eye later during HIFU treatment when cavitation had further accumulated.

Such treatment may be used for example for remote incision-less phacoemulsification, capsule-sparing regional bulk or gradient lens softening and cataract liquefaction for presbyopia, or in vivo US elastography or OCE lens stimulation techniques.

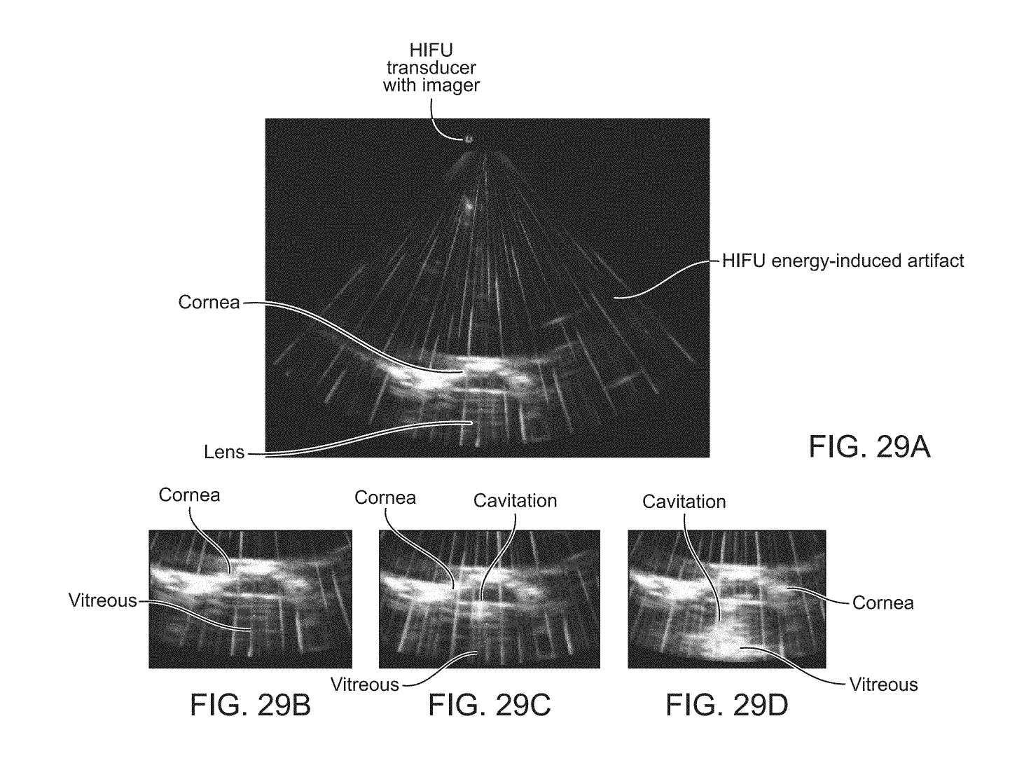

FIGS. 29A-29D show the results of Experiment 14. Using the HIFU parameters described in Table 3, non-thermal cavitation was induced in the vitreous fluid. FIG. 29A shows an ultrasound image taken by the ultrasound imaging system used to monitor the effects of HIFU therapy. The transducer was located above the eye and HIFU energy beams were focused on the eye below such that the beams were directed towards the vitreous through the cornea. FIG. 29B shows an ultrasound image of the eye during HIFU treatment prior to the generation of cavitation. FIG. 29C shows an ultrasound image of the eye during HIFU after cavitation began to occur. FIG. 29D shows an ultrasound image of the eye later during HIFU treatment when cavitation had further accumulated.

Treatment of the vitreous body using HIFU may be used to liquefy tissue, cause gradient or bulk softening, or make the vitreous proximal to the scleral more compliant to assist in presbyopia treatments. Additionally, cavitation in the vitreous fluid may be induced to delaminate the vitreous, as in the case of posterior vitreous retinal detachment, or for pulverization of floaters.

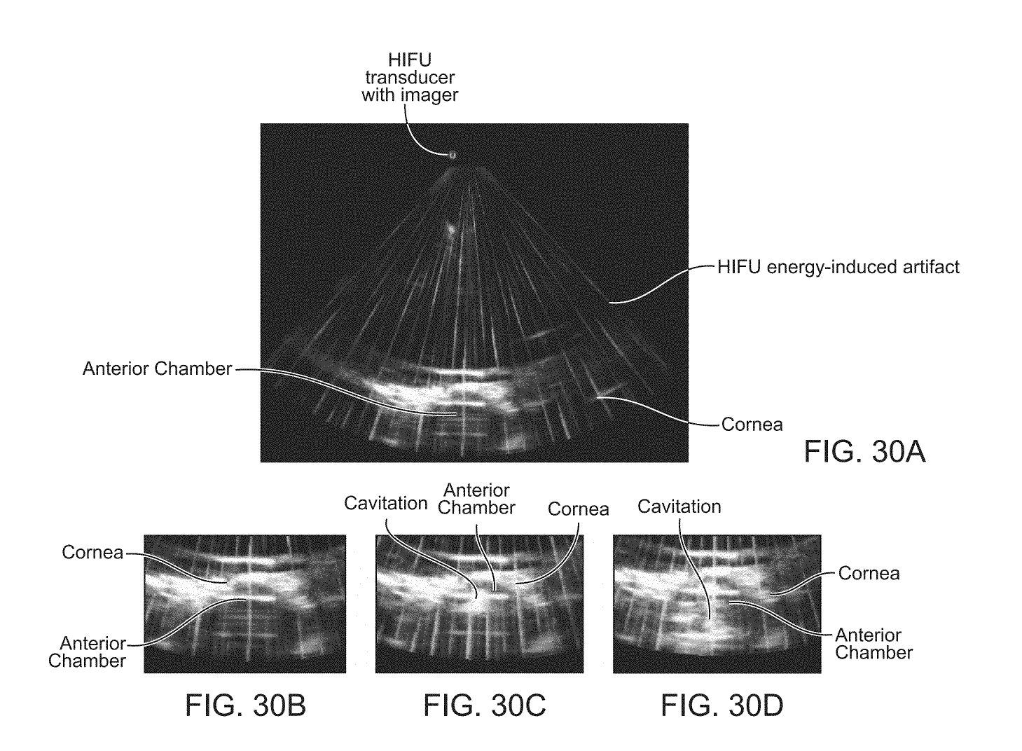

FIGS. 30A-30D show the results of Experiment 16. Using the HIFU parameters described in Table 3, non-thermal cavitation was induced in the anterior chamber. FIG. 30A shows an ultrasound image taken by the ultrasound imaging system used to monitor the effects of HIFU therapy. The transducer was located above the eye and HIFU energy beams were focused on the eye below such that the beams were directed towards the anterior chamber through the cornea. FIG. 30B shows an ultrasound image of the eye during HIFU treatment prior to the generation of cavitation. FIG. 30C shows an ultrasound image of the eye during HIFU after cavitation began to occur. FIG. 30D shows an ultrasound image of the eye later during HIFU treatment when cavitation had further accumulated.

Such treatment may be used for capsulorhexis, in one example.

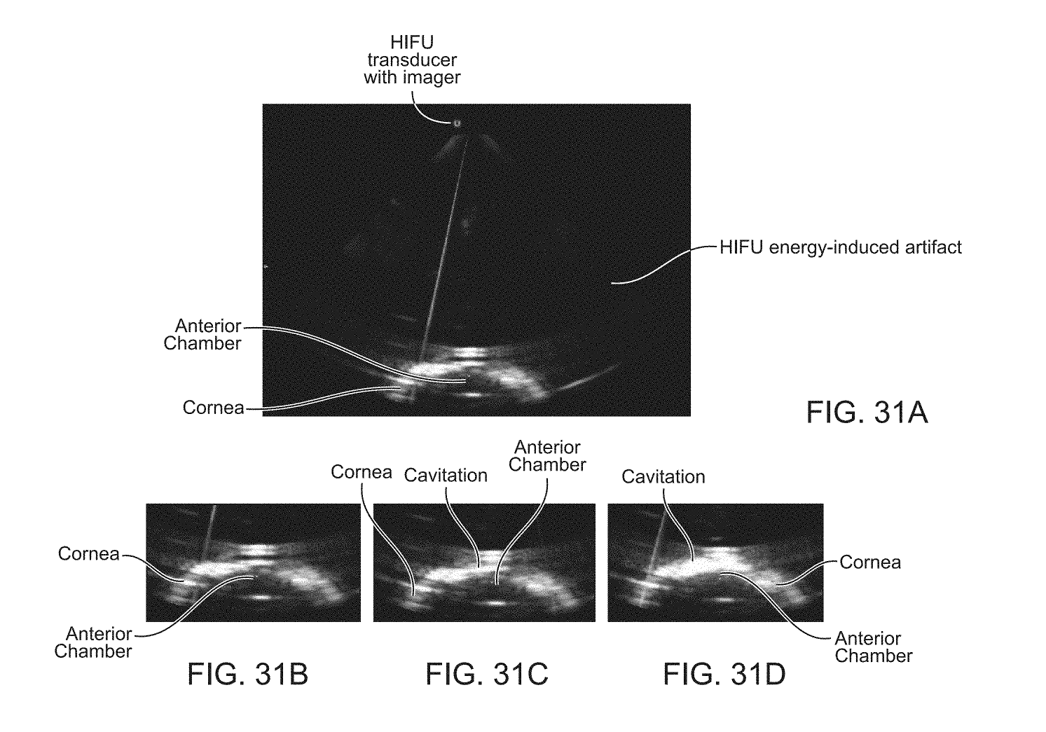

FIGS. 31A-31D show the results of Experiment 19. As in FIG. 30, HIFU was able to induce non-thermal cavitation in the anterior chamber. However, PRF and cycle number parameters were varied such that despite a lower PRF the treatment was still able to induce cavitation. FIG. 31A shows an ultrasound image taken by the ultrasound imaging system used to monitor the effects of HIFU therapy. The transducer was located above the eye and HIFU energy beams were focused on the eye below such that the beams were directed towards the anterior chamber through the cornea. FIG. 31B shows an ultrasound image of the eye during HIFU treatment prior to the generation of cavitation. FIG. 31C shows an ultrasound image of the eye during HIFU after cavitation had just begun to occur. FIG. 31D shows an ultrasound image of the eye later during HIFU treatment when cavitation has further accumulated.

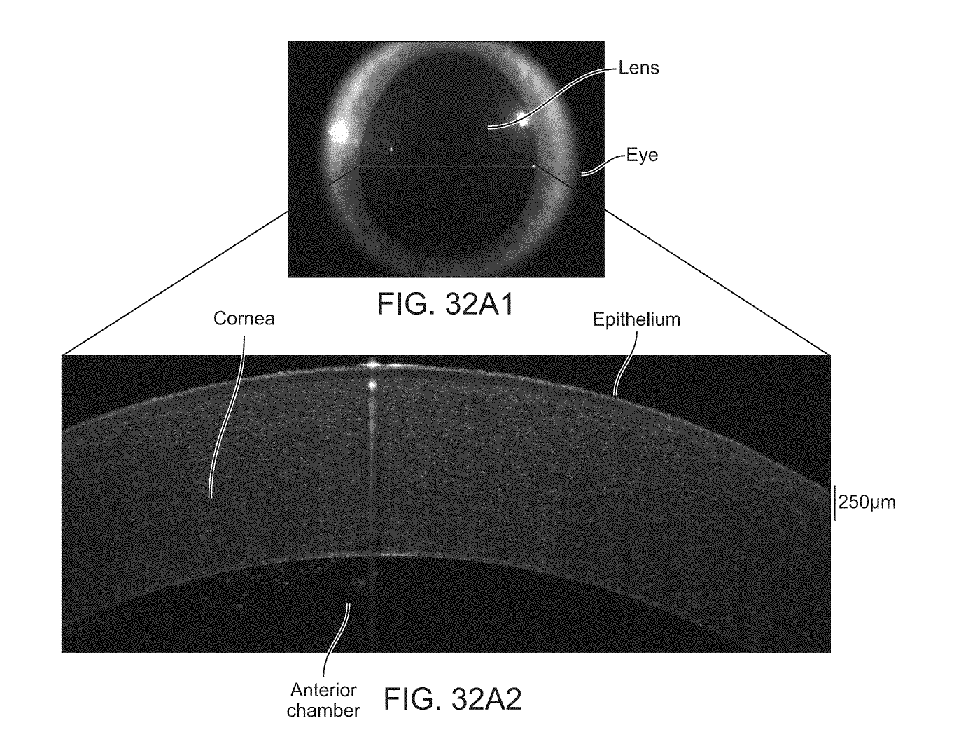

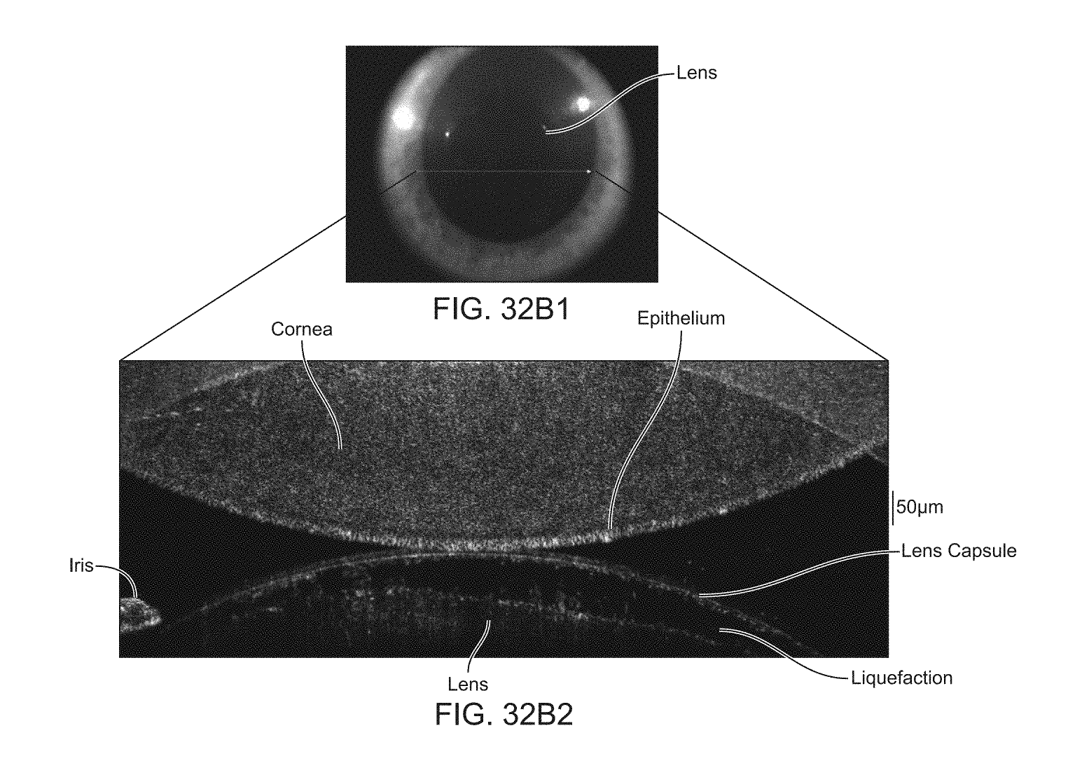

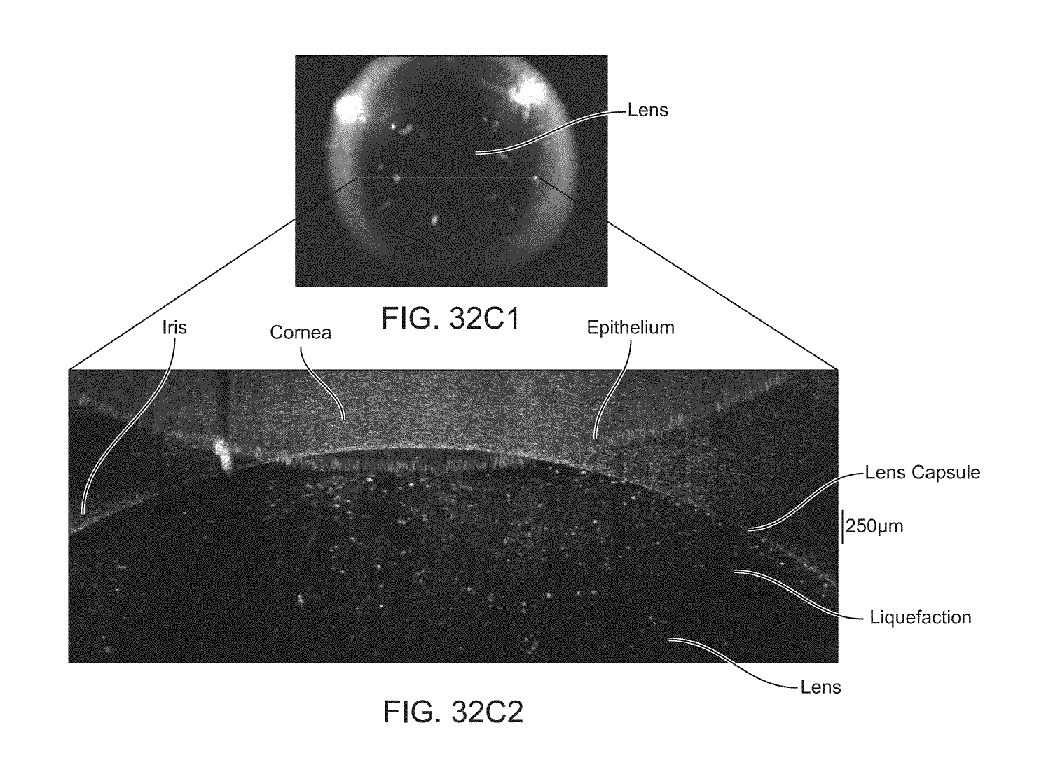

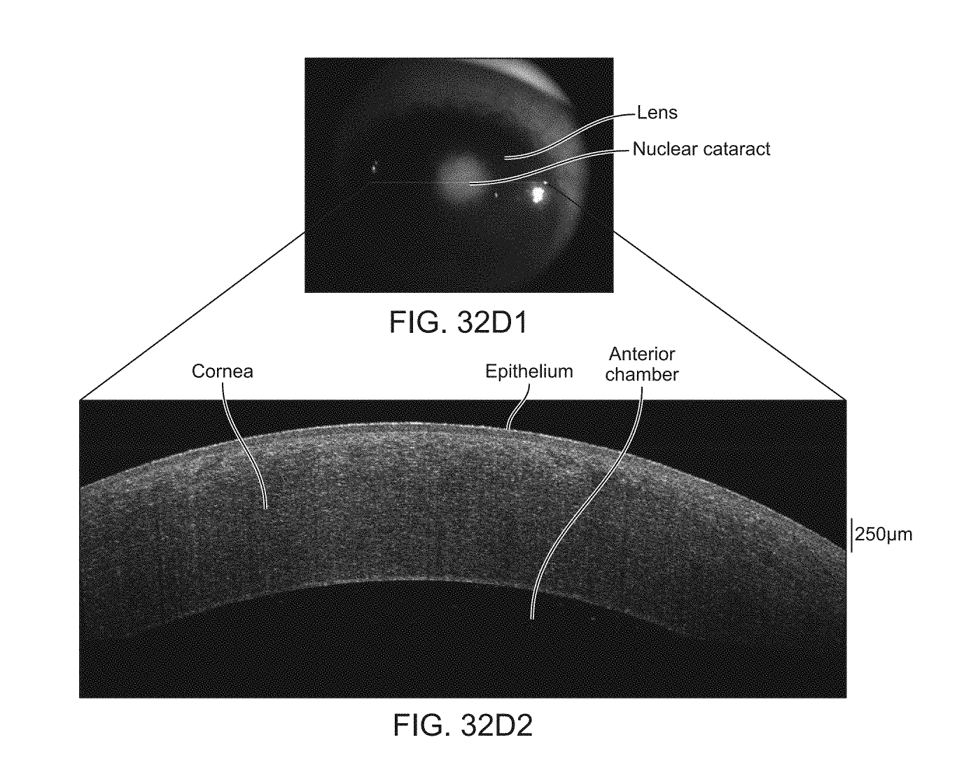

FIG. 32A1 shows an eye treated at the lens. FIG. 32A2 shows an OCT cross-sectional slice taken along the line shown in FIG. 32A1, illuminating the cornea, after treatment. Despite partial liquefaction of the lens by non-thermal HIFU treatment, the integrity of the corneal collagen and the epithelium were maintained. FIG. 32B1 shows an eye treated at the lens. FIG. 32B2 shows an OCT cross-sectional slice taken along the line shown in FIG. 32B1, illuminating the lens. The cornea appears upside down due to aliasing, as will be understood to a person of ordinary skill in the art. The lens has been partially liquefied without opacification and remains transparent. The extent of liquefaction may be controlled by controlling the dose of non-thermal HIFU fractionation. FIG. 32C1 shows another eye treated at the lens. FIG. 32C2 shows an OCT cross-section taken along the line shown in FIG. 32C1. The cornea appears upside down due to aliasing, as will be understood to a person of ordinary skill in the art. Non-thermal HIFU treatment induced full sub-capsular lenticular liquefaction but spared the cornea and epithelium. FIG. 32D1 shows another eye treated at the lens. FIG. 32D2 shows an OCT cross-section taken along the line shown in FIG. 32D1. Thermal HIFU treatment was used to create a nuclear cataract by inducing coagulation of the lens during treatment by prolonged exposure, for example about 10 minutes. Treatment was able to reach the lens while sparing the cornea and epithelium.

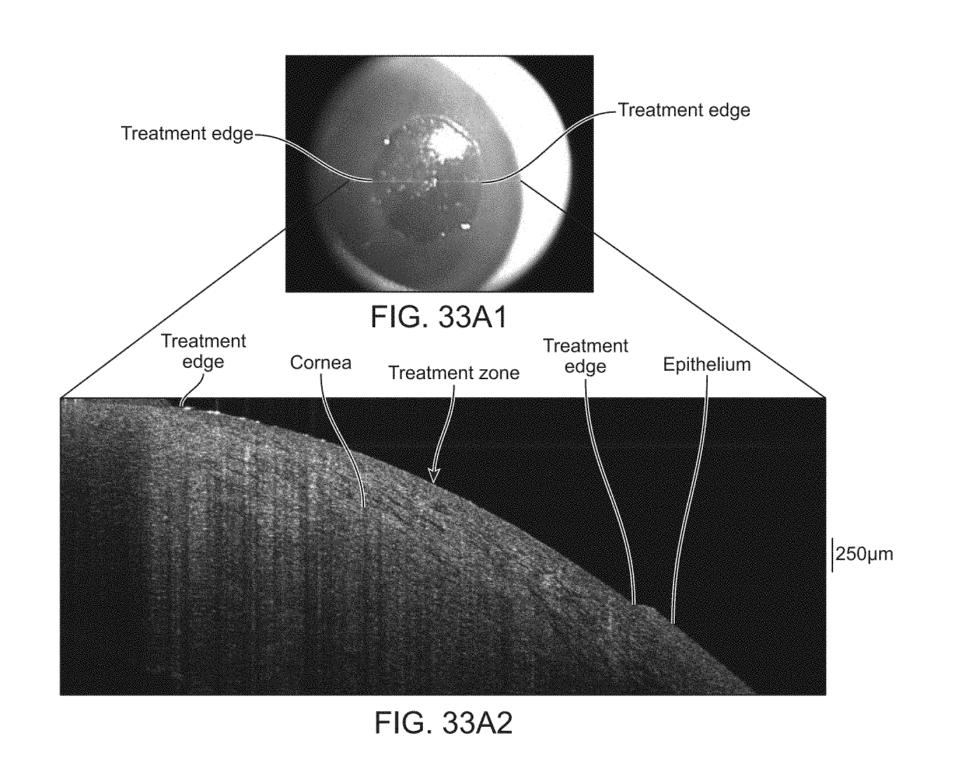

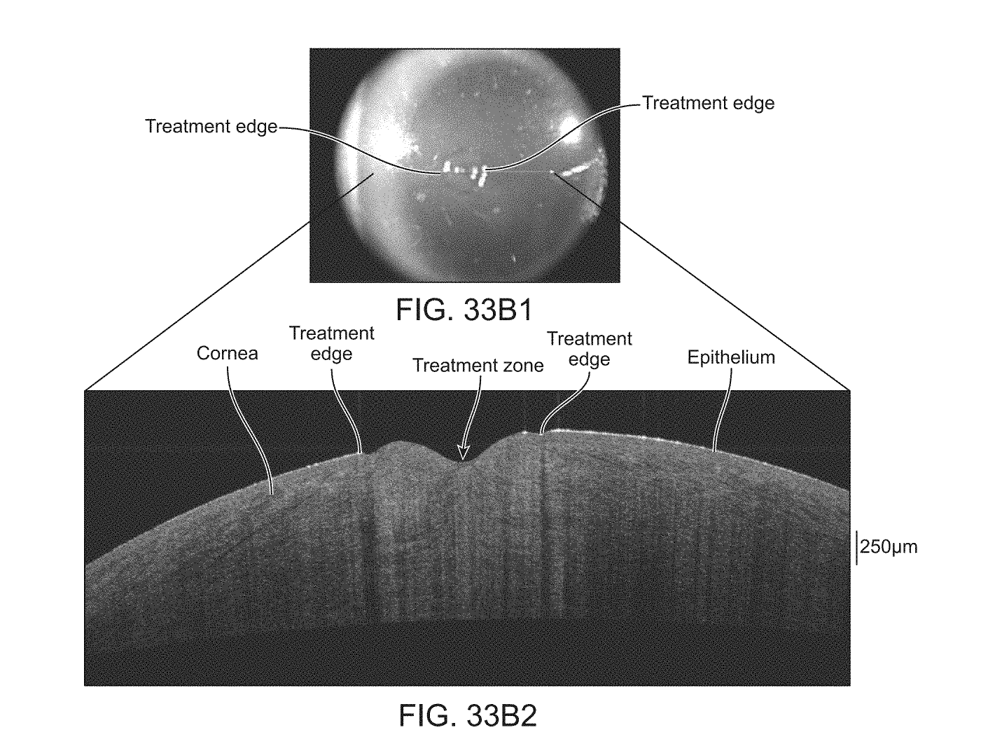

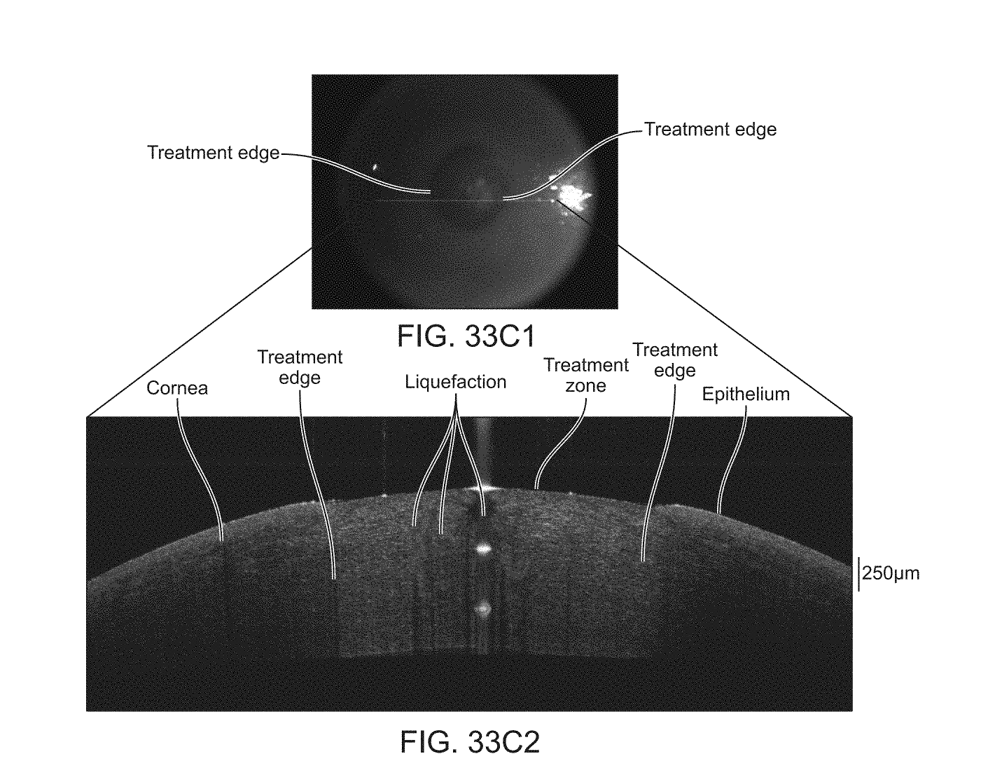

FIG. 33A1 shows an eye treated at the cornea. FIG. 33A2 shows an OCT cross-sectional slice taken along the line shown in FIG. 33A1 after treatment. Non-thermal HIFU treatment induced epithelial erosion with a uniform homogeneous pattern and clear treatment edges demarcating the treatment zone. Treatment did not induce opacification of the cornea. FIG. 33B1 shows another eye treated at the cornea. FIG. 33B2 shows an OCT cross-sectional slice taken along the line shown in FIG. 33B1. Non-thermal HIFU treatment was used to smoothly erode the treatment zone made up of the central 3 mm of the cornea. Treatment did not induce opacification of the cornea. Treatment did not disrupt the epithelium or the collagen outside the treatment zone beyond the treatment edges. FIG. 33C1 shows yet another eye treated at the cornea. FIG. 33C2 shows an OCT cross-sectional slice taken along the line shown in FIG. 33C1. Non-thermal HIFU was used to erode or fractionate the cornea within the treatment zone defined by the treatment edges. Areas which appear darker inside the treatment zone have been liquefied. Liquefaction may be controlled and direct to discrete areas of the tissue. A volumetric liquefaction ratio of at least 10% and as much as 25% of the total lens volume may be feasible based on time of treatment. Not all of the liquefied areas have been identified to provide better clarity of reading the figure. The cornea and epithelium of the tissue surrounding the treatment zone was spared from treatment. Such treatment may be utilized for presbyopia softening and low grade cataract treatments, for example. Capsulorhexis at 100 um width may be feasible with real-time imaging guidance.

The processor as described herein comprises a circuit to process signals as will be known to a person of ordinary skill in the art, and may comprise logic circuitry, a central processor, a microprocessor, random access memory (RAM), read only memory (ROM), flash memory, a field programmable gate array (FPGA), an application specific integrated circuit (ASIC), programmable array logic (PAL), video display circuitry, touch screen display circuitry, a touch screen display, and combinations thereof as will readily be appreciated by one of ordinary skill in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for treating tissue of an eye, the system comprising:
an ultrasound transducer array configured to generate a plurality of high intensity focused ultrasound (“HIFU”) pulses comprising a negative acoustic pressure within a range from about 10 Mega Pascal (MPA) to about 80 MPA; and
a processor coupled to the ultrasound transducer array, the processor configured with instructions scan the plurality of pulses to a plurality of locations to treat the tissue of the eye, wherein the processor is configured with instructions to treat the eye with the HIFU beam to soften the tissue with a temperature increase to more than about 50 degrees Centigrade.

2. A system as in claim 1, wherein the tissue comprises transparent tissue and the processor is configured with instructions to scan the ultrasound beam to the plurality of locations to soften the tissue without opacifying the tissue.

3. A system as in claim 2, wherein the processor is configured to soften a target region of tissue with the plurality of pulses, wherein a duty cycle of the plurality of pulses within the target region is within a range from about 0.1% to 1%.

4. A system as in claim 2, wherein the focused spot comprises a cross-sectional size within a range from 50 um to 200 um.

5. A system as in claim 2, wherein the processor and the transducer array are configured to overlap the plurality of pulses at the plurality of locations.

6. A system as in claim 2, wherein the processor and the transducer array are configured to deliver the plurality of pulses to the plurality of locations without overlapping.

7. A system as in claim 2, wherein the high intensity focused ultrasound comprises frequencies within a range from about 750 kHz to about 25 MHz and optionally within a range from about 5 MHz to about 20 MHz.

8. A system as in claim 1, wherein the transducer array and processor are configured to provide a plurality of pulses to a plurality of separate treatment regions separated by a distance, wherein a duty cycle of each of the plurality of separate treatment regions comprises a duty cycle less than a duty cycle of the transducer array and wherein the plurality of separate regions comprises a first treatment region receiving a first plurality of pulses and a second treatment region receiving a second plurality of pulses, wherein the treatment alternates between the first plurality of pulses to the first region and the second plurality of pulses to the second region to decrease a duty cycle of each of the plurality of treatment regions relative to the duty cycle of the transducer array in order to decrease treatment time of the first region and the second region.

9. A system as in claim 1, further comprising:
an imaging system to view an image of the eye during treatment, the imaging system comprising an optical coherence tomography system or an ultrasound bio-microscopy (UBM) system; and
a display coupled to the imaging system and the processor to show the image of the eye during treatment.

10. A system as in claim 8, wherein the imaging system comprises the UBM and wherein the ultrasound transducer array and the UBM are arranged to detect field perturbation of the HIFU beam within a field of view of the UBM and wherein the processor and the display are configured to visibly display the field perturbation on a real time image of the eye shown on the display.

11. A system as in claim 8, wherein the display and the processor are configured to show a plurality of targeted treatment regions on the image of the eye on the display prior to treatment with the HIFU beam and wherein the processor is configured to scan the focused HIFU beam to the plurality of targeted tissue regions and wherein the processor is configured with instructions to display the image of the eye to view the image of the eye and define a pre-determined treatment region to treat the tissue with the plurality of pulses.

12. A system as in claim 1, further comprising:
a display coupled to the processor to show the image of the eye prior to treatment, wherein the processor is configured with instructions to receive user inputs to define the plurality of targeted tissue regions on the image of the eye prior to treatment with the ultrasound pulses.

13. A system as in claim 12, wherein the processor is configured with instructions
to register the plurality of target tissue regions defined prior to treatment with a real time image of the eye acquired during the treatment and to show the target tissue regions of the eye in registration with the real time image of the eye.

14. A system as in claim 12, wherein the imaging system is aligned with the ultrasound transducer array, and wherein the processor comprises instructions to direct the plurality of pulses to the plurality of treatment regions in response to registration of the real time image of the eye with the image of the eye in response to movement of the eye.

15. A system as in claim 12, wherein the processor is configured to scan the ultrasound beam to the plurality of locations through an optically non-transparent region of the eye, the region comprising one or more of an iris, a sclera or a limbus of the eye and wherein the imaging system comprises the ultrasound imaging system and wherein the plurality of treatment regions are visible on the display and imaged with the ultrasound imaging system through the optically non-transparent region of the eye and wherein the target tissue region comprises transparent tissue.

16. A system as in claim 1, wherein the processor is configured to scan the ultrasound beam to a plurality of locations and wherein the transducer array comprises a phased array configured to scan the ultrasound beam to the plurality of locations and an actuator coupled to the ultrasound array to scan the ultrasound beam to the plurality of locations.

17. A system as in claim 1, wherein the transducer array is configured to focus the spot to provide a negative pressure within a range from about 10 MPA to about 50 MPA.

18. A system as in claim 1, wherein the transducer and the processor are configured to focus the spot to a plurality of locations to soften the tissue with an increase in temperature of no more than about five degrees Centigrade.

19. A system as in claim 1, wherein the system is configured to focus the spot to a plurality of locations to soften the tissue with an increase in temperature of no more than about five degrees Centigrade.

20. A system as in claim 1, wherein the processor and the ultrasound array are configured to decrease a modulus of the tissue by at least about 5% without inducing substantial increase in light scatter of the tissue and wherein the increase light scatter of the tissue is increased by no more than about 5% as measured with a Scheimpflug camera and wherein the increase is measured pre-operatively and post-operatively.

21. A system as in claim 1, wherein the processor and the transducer array are configured to decrease a modulus of the tissue by an amount within a range from about 1% to about 50% and wherein the decrease in modulus remains stable for at least about one week post treatment.

22. A system as in claim 1, wherein the processor and the transducer array are configured to soften the tissue without substantially changing the index of refraction and wherein an amount of change of the index of refraction comprises no more than about 0.05 pre-operatively relative to post operatively.

23. A system as in claim 1, wherein the processor and the transducer array are configured to soften the tissue without substantially changing the index of refraction and wherein an amount of change of the index of refraction comprises no more than about 0.01 pre-operatively relative to post operatively.

24. A system as in claim 1, wherein the processor and the transducer array are configured to decrease the modulus of the tissue by an amount within a range from about 1% to about 50% without inducing an opacification of the treatment region.

25. A system as in claim 1, wherein the processor and the transducer array are configured to focus the beam to a plurality of locations in a three dimensional pattern in the eye and wherein the transducer array is configured to focus the beam to a plurality of different locations along an axis of propagation along the ultrasound beam and a plurality of different locations transverse to the ultrasound beam to define a three dimensional treatment region.

26. A system as in claim 1, wherein the processor is configured with instructions to soften a lens of the eye to increase accommodation of the eye and wherein the processor is configured with instructions to soften a sclera of the eye, a vitreous humor of the eye, or a limbus of to increase accommodation of the eye.

27. A system as in claim 1, wherein the processor is configured with instructions to treat floaters of the eye.

28. A system as in claim 1, wherein the processor is configured with instructions to treat a refractive error of the eye with heating, the refractive error comprising myopia, hyperopia, or astigmatism, and wherein the processor is configured with instructions to treat the refractive error with a pattern of energy applied to a cornea of the eye to provide a temperature rise to at least about 50 degrees C., and wherein treatment of refractive error is combined with softening of tissue.

29. A system as in claim 1, further comprising a patient coupling structure configured to couple the eye to the ultrasound array.

30. A system as in claim 1, wherein the processor and the transducer array are configured resect tissue with a three dimensional resection pattern.

31. A system as in claim 1, wherein the processor and the transducer array configured to spongify tissue, to mircoperforate tissue, and to emulsify tissue.

32. A system as in claim 1, wherein the processor and the transducer array are configured to heat the tissue to greater 50 degrees centigrade to provide a thermal treatment.

33. A system as in claim 1, wherein the processor and the transducer array are configured to provide a focused sub-surface treatment selected from the group consisting of myopia, hyperopia, astigmatism, presbyopia, spherical aberration, keratoconus (KCN), phacoemulsification, infective keratitis (IK), CNV, cyclo-sonocoagulation, glaucoma, floaters, vitreolysis/vitrectomy, lens epithelial cell (LEC) lysis, capsulorhexis, glistenings, tumor, sonothrombolysis/vascular obstruction, posterior corneal surface reshaping, posterior capsular opacification, capsular polishing, extravasation, posterior vitreous retinal detachment, posterior continuous curvilinear capsulotomy (PCCC), and anterior continuous curvilinear capsulotomy (ACCC).

34. A system as in claim 1, wherein the processor and the transducer array are configured to direct the ultrasound beam through a tissue of the eye selected from the group consisting of a pupil, an epithelium, a conjunctiva, an iris, a capsule of a lens, a sclera, and a cornea.

35-106. (canceled)
